SEQUENCE LISTING (111 sequences)

SEQ ID NO: 1 - DNA, 393 bp, Homo sapiens
acgaacgaaagaaagaatcccaactctgcgcaggtggattcataggcgaagcgaggatattgtggaaattcagaaggaaaagataaaaaacaggcgctaggatcagatgacggtgataggctgctcggcacacaaagggagcgtagggcagggtttacggagcaagcctgcagcgaatggggcacagattgttccgagatccagtcgttttctcagtcagatctacgcgaagggaggggaggggaggggcgggcaggggagcgtggcgggaggggctgagcttgggggcggggggatttctgatcagtctgatgcaattccaagcgtgctgcaaaggaactccaaggcgcccgcatcaccatcgccacccacccttcccagatggwgctgttt

SEQ ID NO: 2 - DNA, 952 bp, Homo sapiens
aaagcctccatccgcccctctcgtctaccgccgtccccttccagacacccccagtcctgccagacacatggcactctccgagctgggaaagctaacctggagctggcagagggtcctagcccacctcaagctgctagcccgcgactagcatccatcagccggaattccgctcataaatggggtccccctccctgtccagccgtcgcctcctgcccccaccttcttgcgctgcttgtgcgaggagtcgtgcaggttagagaggcagccagatcccagaatggtgcgcaccgacgctggccagtggaccgacaccagccggtgctctccgagcaagatgcgccgcacattgtccgcgcccatcacccgtacggtgggccgcccgaacagatgcgtcttgtagatgaagccgtatttcctgcgcttcatctgcaggaacttcctccgctgtgggaggaaggcggaggagagaagtgggcatgagggggcgccggggagcgccgcgctcccgccagccctgctcctagccgcaatagcatgcctcccgggggcgcctaccccgacttcagcagaagcccagagccgcgccgggctccggggaagcagcctgtcccgccccaccctcccttacctgcagtaccatctgcaaggtttcccaaagaaggggaagcccatagtcccggggggcaatgggagggcacaactgcggtcgcggccgctcacgcagtacaggtcccagagcttgatcgcagccaggaagagcagcagcggcagcacgaaggtgcagagcgcactggccagcagcgccgggagccccatggcgcgccgcgacctcccgcgccacctgccgccgccgccagcgcttcaaaccccacggcgctgccgctttataggcggcccaggttgctgcccacgttaccttggtcagacaattagttcacccaaagttcatctt

SEQ ID NO: 3 - DNA, 166 bp, Homo sapiens
aagtgggaagtgcaaattctctttgacttttaccccgaccctatagccccaactaagaataccccaggcccggattcaaacaaagagaagaaggcacgggcactcctaggaaaagaaaggcatcgggaggagcacgcgcctcagaaaccctaaaagcagagtgaaa

SEQ ID NO: 4 - DNA, 49 bp, Homo sapiens
tattttcaagtgtttagtggagaaaagctgagacctattcawtakattt

SEQ ID NO: 5 - DNA, 1476 bp, Homo sapiens
tggggccttccagccataaacgcttctcttttttcccccctctctggttagttcctcttcccttttcttgtctgcaccgcgttcatgccggactccgcggggcccggtgcgggtggaagcggagcgcggcaggacaggtgcgggagccggggcgcacctgtgcctgcgcgcgcgcggcgtccagcagggggcggtccgcgccggtccccgagctcctctctgagcgccccgcccccggtccggaggcccaggtggcagcgggggcgcgggagggcgcgtcgggccggtcaccggcagggctcagaggcgcggcggcgcacgggccgtccgctctgacgcgagcgggtcctccctcctcgacggccctcgactcctccccacacctgggaggggagtggtgcggcgcggcctcctcccccggcgctcgcaactcctgtccggccgtagctgcgccgccgcggcgggagtaaaggtcgcgccgccgggagcgagccggccgcggcgcctgcgggaagccggcggggcaggtcggagaagagcgagaagatcgagaaactccaggccagcccgggaacatggcgccaggcgggccagccgcggactgagagccgcggggcagccaggagccggggcccgagccccgcccggcccgggccatgtcggtgggcgagctctacagccaggtgagcgcggccgcgagggaggcgccccggggccggggtggcgcgctcggggccacctagtactcagggtcgccttctccctcggctactggaggcgggaggtgacgggagacccccctccgcggtgtcctcctcgtcactgtcctcggagtccctaacttcggttggcggagaaagtttggcggttccgggaacgcccgcactgaccggccacccggctcctagaggttccgcagcgccacggctcagcaccaagccagggctgtcgccccctccccgccgcgcccctcactttcgttgcctgggctcggctccaaccgagttccccgaacggcagggcgaccctgccagctctgagcgccgtgcgcgggacgggcccccgcggtgctcccgctgctgtcccctgtccccgccgtggaccccaccccaggggctctgcggcgccggctctcgagggcgcgcctccgaggcgtcctttcctcccgcatacccacaacagggaaccctggacttggagaggcgggggttctggactcggtggagagggaggagacaacctaggcgtgagggcgcggcggctcgatcgagtgagggtaagcaatgggtagaggggtgcgccgttatctccggagactccagcgaagagcattgtgaatctgctggggatggatgttttgacgcggcccgtttgttacaaagagggctccactgactgcggggagggggttggagtaaagtcgaaccgagagcagggtgtgagtttcaggtcgccctcaaggtcagaagggatgtg

SEQ ID NO: 6 - DNA, 738 bp, Homo sapiens
aaaagactagggaggaaaacacccaaccaaaactccgccccactgtcacctgggtccagtctcgggacaaatttcaagatctcccttttcgcaactgttttcagaaaataaaaggactaactgtggctgtcaagtgctatgaataaaaagagcaaagtagcagggcaaagccgaggcccaggcaggccccacgctgcccgctgcggaccaccttcagggtccagtggcgcccggtcccgagtccaggagccttttcgtcgcgtctcacactcaccttgctgcttagatcctccttgtgtggggtcgaattgtcggtggggatcagcacagagctgtcctctggtcgcgtccctcggctgggcatttccaccgagcgcaagacacgcaggatggctcagccctgaaagagcctacggcattagccacagcgcatgcgcgcttccagccgcgctcaccctttcttttcccgcgagtcttaccaatagcgtctgcgcgctcacttgaacgcatgcgcaggccgggtctggcctggggaggagtttcgcgcctgcgcatggctgcttagggagcgagtaaacagtctggttgcaaaatctgaggttcctgtactggcctcttgtggttgagatgcagaaacgctcaggtggtttcttcatgacctcttatctagtatactacgagttctgtgcgccctccagtggtaaatatgaagaatagtcgtgcaatagaaacagcgct

SEQ ID NO: 7 - DNA, 823 bp, Homo sapiens
agccgtactcctgcaacatccgaggtgcgcacgctggcccaggatcctaacaaattgctagtcggtggctatgagtcttagattatcagggactctccgggctctgcggacacgcagaacgcaggcgccacgcaagccggccccgccctttcacccctccccgcgctcccgggccccgggcgcgccgcctggaaccctcccggtcctggctgggggcgggcgaacgaggccccacctctgccgggagcgggacgagcgcgcaggcgcagtctccccaggttgtagacgctgcggcccggcccggcggggtgagttgagaacgcggcgctttggtcgcagcggcggtatggtcgccgcagtttctcggtcttcgcttcggcgaccttgacccccttgtgggtgggcgaggctggaagagaggctccccaccccactcggcttcctgatccctgggggcgccgggcggtcaccgagggccgtgtgagcgccgctctgccggctccgggtccatgcggctgcgggagaagccctgggcgtcggggctggcctgggaggggcccgagtcccctggtgacctgggcgcgcggcggccccacacctttccgtccgcctcatcgcggcgcatttctcttcacatcctcggggtgtgtgtgtgttggtgaccggaggaagcctgtctaatgacttttctggaagcgtcgtgtattaagagctccggggccggactgcttgggtccgcatctccatccaccgcttactggctttgggacctctgcaagtcacttcacttctctttctcagtttcaagtgtaaaatgcggaaa

SEQ ID NO: 8 - DNA, 388 bp, Homo sapiens
aaaaatgaccttatttctacttgcacaagccacagtggctaggcaggcatacaggatcctgatgtcccctcaacaatgtcagcgctcataatggctgcaacagtgaacatccacttggcatttcctatgtgagcaattttgtatccacatggttggctttatgagcaagtagcctgaaaagtcaaaatctgctccccaggctcaatttcagggaaacattcagccaggtatctggactgagaagtggccaaccttcagcacaatctgcttagggtggtttttggttggcagtgctatcaggaagaatgctgagagccttttgcccaagattgttcttttcttattccatgcagagagcctgtcataaccacagctctcttgtggtttt

SEQ ID NO: 9 - DNA, 238 bp, Homo sapiens
aaaaacaatgactgagaactgaaagtgagataagatacggtcacgattctctgccaagtgataaaagcctgagggacgctgccagtgccaggtgggagcggaaagagggatgcttggtagaggccagagacacgaaaagcagcgacagcctcgtgctgccctggccacttaggagacaagggggagccaccccatcagaaccgcaccgggcacccccgtggtcggcccgtrctggctt

SEQ ID NO: 10 - DNA, 286 bp, Homo sapiens
  misc_feature (28)..(28): n is a, c, g, or t
taaaagaaacaaaaagccaagaagccgntatggagcgggtgtgggggcctgtgaccttttgtctagttgccatcctccgggaccaacaaggggtacatcacataagggacgcggcggctcaaatgacatgaacataggagtccgcattctctgagatgtcattcctccaggaagacccaggaaccgtgaatagggcggggtggtgggnaatatctgaagcgaggtttcctgggaacgcgatgccactggccaggacgctggagagggcagcggccgtgaagaatcc

SEQ ID NO: 11 - DNA, 1052 bp, Homo sapiens
taatctttccccaaattatctgtgaataaactaccttcagcagccgctagactagagtagatcaaagttttccctatacttgagaatgtgatccccactgaaaaggaacagaaactgatcatccctgccactggagccagtccgcggaatctccccaaatacgcctgatcgccagatgaagaagggacacttctccgggcagcaaaggctagaggtatcttacctctgcaggctagaggaggggtggaggaggcgggaggacactggcacgtaaggaaggaaattccgtcttcagcaggcactggtgggacccgaaacctaggtccccgaaaagcctcagaacccagggaggttccccgagggggacgccaagcagggccaagactctccgcgcccggccccgcctctcttgccgccaccggggacccgggaccgcggacagagacagttgaggctgagcgagactccgcggtcccagccaggtcctacaggagctcacctgagagtgtcgcctccgccctcggctaccacggtgcatcccgcggagcctggcggagggtgcagtgacgccgccccgaccggccccagggctgcccggcctggcctccccgctccggggacgcaccgggtgccctcctcgcaccggctccggggatccgccggtctgctgggaaggaagacaaggcggaaaggctgcctcccctccctcagggcaagccaccccccgagctgcggctccctgcagttctctctggcgcaggtctggccccgctccccggcccgggagtcacttcgtcaggggcggcccagtcgccattacagcccaacaactgacacggaggaggagcggcgccagcggcagcggctgtggcggcggcgcgaggcagggcgggagcagtcgggcccaggcaaacctcgcgagaactgtcccgagacagtgccacaggcatgccaaccttcggttagggccatgttgagtgtggccgaaaggtgggagcgaaataaaacaaggagcagaaccgcgaagaagccatgtttggtgcgtagaggtt

SEQ ID NO: 12 - DNA, 750 bp, Homo sapiens
cctttccgaggccccgcccccaacgggtggcgcgaggcggggcgtaggtgagcgctgcagagaggctgcgccttgggccgcctgtcgcctgtgcgcctgcgcgcggcgccgaggggacgggktccgactcagaaatggcggcctccatgttmtacggcaggctagtggccgtggccacccttcggaaccaccggcctcggacgrcccagcgggctgctgctcaggtaatgccttccagtccgctgtcatctcgagggtcggcaggagaaagggtgaggtgtgaggggtcccgtggcccggctcggggtcacttcgccggcagtgacattttctgggtgccctgcaaggccctcgggcgacttgcctgggagtgctgctcgccaggactacgagcgagcccatgtttgcgcggctctgacccagcccttagtccgcgaggatccccggcggcccggccgtggctgctcctgcagtttctaagagatgggcgggagcaggtgggctctgagtggaggtgagggggtcaggacattttctgctgccattccggcgtctggcacggtgggctgagacttgcgccgcccctttttgggcaggggggaatgcccttcaaaagtacttttgtgtttcttgggagctgtcwtaggtgggaatgtcagaggaccagcggtgcttactgacttgccttttgtggggcaaggtagagaagtttcccctctcctccaagtagttgtatactcttaaaggtgt

SEQ ID NO: 13 - DNA, 1197 bp, Homo sapiens
taagtacaacattcaagcaccacagcacatgcctaagggaagcattagaagagattagaaggaaaaggccgtggagacctcaatccctcctcctcccaccgcgggcctggcctccaccgccacctctgccaggcgggcatggaaggggcgacctctccgatgcgcactgccgggcgcgaagccgcccgcgctccctcccccagcgcggcgcgtgccactggcccgggaggcccaggcgtgggcagccgggtacctagctcggccacggtagccgcgcagccggacagtaggaatttgctcgctcggggccatctctgggtcagcggcaaaagcctctcctcctcctccgggacggacattcagcagtagcgcaagacgataacgcactccttctcgccgctgcgctgcaccgcgccgcggcggccgcttycctgccagggcggccccttttcaaccggcgagtggccgggtgaggaccatwtccctcgttcggctcggccgggtggacccgcaaccttcctagcagtcccagctggagtggtccagctgccccttacaccccagaatagaaaaacacagcggagcccaggaggttactgggcaatgtagtccgccgggctccgctacgtctctgattggccggccgcgggtccctctgcggctccgcccccggcctccatggcaacgcggctggttctcgcccgtcagtcctagcccggccctgcccctccttgcattttttycgcgctggctgagattcaaagagaagtggaggtgggagggagcgacaatggaaaaatcacctgaaaactgggacagaggaaggaagctacagttacgaaggagagctgcaaaagttgcagcagaaaggttgggagtcccgacaggttccgtagcccacagaaaagaagcaagggacggcaggactgtttcacacttttctgcttctggaaggtgctggacaaaaacatggaactaatttccccaacagtgattataatcctgggttgccttgctctgttcttactccttcagcggaagaatttgcgtagacccccgtgcatcaagggctggattccttggattggagttggatttgagtttgggaaagcccctctagaatttatagagaaagcaagaatcaaggtatgtggtcgtggcagacggggtctccagaggagacaatgctatcttttt

SEQ ID NO: 14 - DNA, 230 bp, Homo sapiens
acaaattcctgctctctcggcccccgtcgctgccatctgggctctcctttccgcgcttcattaggcgctgccgcggcaggggctgagggcctcaggggacggtcgcctccctgccgggacgccccggccctggcccgcactgtggggcgcagtcacccagccgtcgccgcgtagccaggagcccggctgagcgcgttcgctgcaggagccatcctcagctgggccctact

SEQ ID NO: 15 - DNA, 862 bp, Homo sapiens
agcgcgttcgctgaatttgcaccacccaatccagccccgccgcccggcgccgctccttagacctggaccccggcaccctcccgccggggccgcacttagcagtggaaagtctgtcctcctgaagaagttgcgctccgacctccaagcatcaggtcaaaagtctaactcattctctgacctgccgccaattagaaaacaactgttgccaataaacgtggtcgcgccgcctgtgacctcagccgggacggacccgcgggcgggagcctgcggggcgtgaggcggggtggggccctggctcccctcccccgcccagccgcggcgtctgacgtcccgcgcgtcggcggccgcggagcagcgcagggagccaggcgggctgccggcgggtaagggcttctgcgacccggggacccgtggggcgtaggccgcggagggagagcgccgctgcctggtctggagaaccccgcggagctggcctggcccgacccggcccggcccggtccgcggcgcggtggtcgagggcccgcggcgccttctgggagcgggagggccgggaagccgggcgttggtggagggaggcgttgggcttctgtcgccgcgaagctggcggaaaaggcagtggccagcgagccgcctcgccagttcccacgactcccagggaggtggcgacacccgcttcccgggccacccttttccccttcctgaccggggtccgtgcttgccttcccaggtgggggaaggtggaacactgtccatcccctggacaaaaacgctgcgcctcggggccttgaacccggttttgtttgaaaggaagcaagagaaaataagtgtttttccatttaggtgtgaagaaaaaaatgacact

SEQ ID NO: 16 - DNA, 145 bp, Homo sapiens
ataaagtcattataatggataaccaatttgctctgtgaataaaaaggcatttagatgttatatttataaaaataaaacaaaaacactttctaagccacttttgaaaaatatgtaaattacctagaagtactttttttgwttgttt

SEQ ID NO: 17 - DNA, 269 bp, Homo sapiens
atgtaaccctggtctgcgtagaagtgtgtgttgtttcgcctgcttcccctgtacccaggtcggcggctatagagattgtggacggcgccatgggtttgcccgatttctgctgtaaagaataagactaggtcaggtgacctttcggggcttgagtgtgggaggaacggagacaatcctccatccggctccgttatccagataatcctgcgctctcgggggttttccgatctggatgattatctatctggtcgttcacacctcagctcact

SEQ ID NO: 18 - DNA, 289 bp, Homo sapiens
cagaggactacaacagaaacggctcccgcgaagacacaggaaagaagacagtcagaagcacccagatacactaaaagtaaacatacaaccccaaagaaaacgcatccccaggaaaaatctgagaggtcttagaacctctagccagcctgactggtcacagttttcccctaagtaaaagtagttcataaagattgagaaaggtggctgtttcttcaaatgctcaaatcccaacaaaagacgacaaggcatacaaagaaacgggaccatggcctgaccaaaggaacaaaat

SEQ ID NO: 19 - DNA, 891 bp, Homo sapiens
cgccggacaacatggaggcagcgccgcccgggccgccgtggccgctgctgctgctgctgctgctgctgctggcgctgtgcggctgcccggcccccgccgcgggtaggtgggcgcaggccggccgggggccgcgggttgctcggacaatggcccgggcggccccgcggccaagtgcgcgggcgccgccgccgtctcggaagcgacttggcgagttgggagcgagttggggcgcgcgcccgggatccccctgccgtccccggcgtccccgccacgcgcgggccctttgtctccccacccgggccctccccggggacgctgcgtcccgggctggcgaggagggcgcccgctccgccaggtgcggcgcgggcgggtcctcacctgcccgagcccgcgggaagccgggagccgagccgagcccgcgctgggcccgtgtggcccgggccgggcaccgagcggggacttggggcgcggaggcgggcctggcccggggagccggttcgcaagcctgtttccagcggccgcgcggccgccccgcgtgctccgaggacgggctgaagttgcagcgagaaagttctgagcccgggcgcggggcggcctggccggtggcgctgctcctgtgagccgcgtggctgtgggtttccaagcggactgacacctgccaggtgcccgggcgacccgaatgccccgtgtggccgccgggtccccagatagtgtccatccccgggtcggctacgcggaggtgacgtggtcccagatcgcggggcagtgggggaggcaggcttgtgcctcagttgtacactcccgtggggagaggtagggcgggggaactttctctccaacttttggtgtttcccagggggtggagttgaggtcaggaggactcaggaaact

SEQ ID NO: 20 - DNA, 878 bp, Homo sapiens
aggatctggggtctcagggatttgaataattggccaggattcggtggggctcagggtcgccattcagatggcggactcgatactgaagggtctggggtcctgaggagtctcagtcccagagttaggcttttggtcgatcgggagttggtctgtcaagggcctggtcttttgtgggtgatgctgacacttggggtctcagtccccgtgggtccgggggtctcagggtcaggttctcaaactccctgtctgatagaggtcctggcctcgggtcccggagagggtggggtccctgggtcggttccgcggggctgtgaaggggtcctgccccaggggcgcggaggccgggcggtaaggggcgcactcacctgcaggcggcgcaggtaggctggcggcacgtagcccgtctcaccactgcgcgcccgcgcggccagccaccagtgcgcgctgcttcgctctagcaccaggaaggtctcgcccgcggcgaacgccagcgcgttgggctccgccgagcggaacgcttacagcgcgcggtacatgaggccgggcagggcaggtgcagggaagcgtggcaagggctgcggcgccacaacgccaggccgggagcgccgagccgcgccgcggttgtcccgccccgtgacacactacgcaggcgcgcgcgcgcccgcccgccgaagccccgccccgggctgtatcgttccacgcatcggagagcagcgcccccgcgcggccccaagaggtgcggcggggctggggtcgacagctcgctgtctcctctggtccctcccggctccaggcccctcatccccggacaccgcaccctcactcctcaccccggcctcagtttcacccgtccagccctctgctccctttcaaacgct

SEQ ID NO: 21 - DNA, 289 bp, Homo sapiens
aatgaagagagacgcggagaactcctaagagagtagatgcagatcaagcagttcgccttcttttatgctttttccttgtgatgcacgtctcttctttcacacttgtstgrgacaggttstctggaaktgggatccagaaggcttatagtgagagtgggactgaaggatggggagtgcgtgcgcgcagttaccggggggcatttgttagaactccggctttggcactagtggggagttggctctagacagaggtttccaggatcctcataggatgacgtggaagggagac

SEQ ID NO: 22 - DNA, 140 bp, Homo sapiens
aggagcctactagattgtgtaaggagggaaactggtgagtggggagtaaaagcctgatgggacttcaaagcaaatacggcttgaagagaactcttgtgactatcaattgatactggcagggccttttgaagatgcatttt

SEQ ID NO: 23 - DNA, 1526 bp, Homo sapiens
ttaaaattccgtgtgagacctgagaacacactgtgaagcggggttcggagaacgacccctcccgcgttccgcgcccagcggggtcgcagggctgcgagcccggctgtagcaaagctttctcggccgcgtcctccctccggattcggtaggccaggctcgggcgcgcccttcccacaccaacaaaccatctttcccgactcagcagaggcccacaggggcgcagccgctgtccctccgcccttggcccagcggcgccgccctggtacgccaggcctgaaggcagggccggcccgcgccacgcagggtctcccttaggcggcgccttagggtgaaatgcggggccaagcctgacctgccggggtgccccgtggcatctctggtgcggaccccgcacgtgccggggagaactggcaggcggccagggcgaggaggcttccagcttgcgccgcactggcctgggcacccgggctgcagggccttttgggccctcccctcagtgaccctgcctgtagcggccccagggccgctttggctcaggcagcagctgccaatgccgctcgaggcccgcggctccctgaggcgagctctgcggggcccagcagtcctcttggctgctaaatggaacgaaaccaggagacccggctttgcactcgtttggacaactgtgtggcctccggagcaaggtacccaaacccttgggcctcagtttcgtcgcagttgttatttgaacccactcggcctgggacgtgccagagcttcacactttaggtcgcggctgcgggcgggaggggcagatgcgacccctgggccggcccctcgggggcgtctagaagcttcggcacctgcatactaaggccggagggggctggcagccaggcacaccgaggcccggccggagttcccgcccggggaacgaggcttcgaaaagaggcacgtgcttctcccagggcctcccgccaggtgctgaaaccccacggcactgagtggaaaccggaagagggaaggcgcccagggcctgccgggccgcggcgccacctgaggctgccgggacgctggagcgtgggggagtggggtgagtgcgctgccgggccgggggcggcgggggacggcgggaggtggccaggcccggcagggtgagggcgctggcgttggccgagagtccctgggcagcggcggcgggaacgcggaaggttccctccacccccaccccaacccccacccccacccccgggccggccacctgttgacgggcaggttctctgagaaccgacttcagctcgcccttctgtccaggaagtttgttcggtttgggtttgggtgttcgtgacacaatcggagcaattagaattgaagcaacttcaggagatttagcaacccgcgaaaatttagcaacgttcggacgtcaacagttttctttctttgagtggtcaacgaaaacttgacgttttctactcataccttgtattctctgcttatactgtaggggcaaaaaagtctactcttagacaaacgtgtgtgaaatgctt

SEQ ID NO: 24 - DNA, 338 bp, Homo sapiens
ctttcgatttggcgtctccctcgaccaccacctctttgtgcagcagcccccgggcagaccctgttccgaggtagcgagggggaaggtccggggcagccgggagggcttgcgggatgcgcgctgggttgggtcgccgctccccacgagccaaagccgcctctcgctctgcccggtctacccccttttccatctgggcgggggtcgctctcccgtgggcacaggggcgccgtggacgcgcggggacttggggccggcagaaggggatgtgctttggggcggccgcgaggccgggggagggcgcccttccccgccggggtccgggtcctctcgggcacctc

SEQ ID NO: 25 - DNA, 101 bp, Homo sapiens
ataaaataataagggaaacaattctataagcaattatacaagagggcaaaaactgcttttggtaagaaaaagctataagatatgaggatgttttattttgt

SEQ ID NO: 26 - DNA, 239 bp, Homo sapiens
aaggggccgcgcgctgcgcgccccagcccgcgggaggtggagccccgtgccccgcacccagcgccccggtccctgggcggccctgcccgggcggcctcgcgcttagggcacccgcagcgaggtttccgggccgtcgactccccttgtatcttgatctagggttttggggttcccgacgcactcattctgcccctaaatccagtcgcgcagacctcagctccgggggaccgagttctggg

SEQ ID NO: 27 - DNA, 655 bp, Homo sapiens
tttattatgtgcaattcaagtccccactgcccgcccgcaagcccccactcatcctcgctgcgggcagggtggcccctgcactttacaagggggtgcaggagcgggagacggtcgtccgaacaccggctccccggcatgcgtagaccggcgggcggagcgggctcactttgcgccaatcctacgagaactcccagaactccgcttccctagtccaacccaagccagagttgcccacacctaagatggcggcggggggcggagtcggcgcggccgcctctgggcgggaccgcggggactatacgtggccgcggggcggtgtcatcgcccccgccccgcccggtccagccagctcggcccgggggcttcgggctgtcgggccggcgctcccttctctgccaggcggcgagtacacctgctcacgtaggcgtcatgaggtctccggttcgagacctggcccggaacgatggcgaggagagcacggaccgcacgcctcttctaccgggcgccccacgggacgaagccggtaagcggcggggtcgtccaggggcttcgagtgggcagcggtaccgggggcggcggtctcttgggccggcatggcccgggtacccgcaccctgcgccggcgggaccggagctgctcatgcgcgaggggacat

SEQ ID NO: 28 - DNA, 1039 bp, Homo sapiens
gatggggggattgcaacctggcccaatgtgtgtgttgcaaccttgagttggcagcgcctctgactgcccactcaggatcaaggctgtggcttgttgggatttttttcctcctttgcccccttgaagtagtaattattactgttattattagaacagcaaggttcggttgaaactttctcgtttcttccaccgccgcctgggagctgtcgttagagcattccacctgaggcccgcggggaggaggcgggcttttttttgtcgcggcggttggcccgcctgtttgttttggtctccttgcatgcacaataaataataattaggataataatggataatcccgggcgaaggcggcgtggtgccggaggggtctgagcggcggctgcagcggggcggccgaagggaggggaaagaggagaaaagagttgtaagttatatttaccttgctcgcgaggagtgtgctggctgaatctcttccctctctgagtcacccagagtacagaaacagagggagagaggactagattatgaatatgacttcattgatgacagccgtcccctcctcttcctaaactcgggctccgcccagcgtcccaccctcgcctcctccgcgggcgccggaagacgcgccgcagttggccccgcgcgccgctcctccctgcccgtccgggcgcgggttggcgggacgcgcctgtcactcgctcagctcagcgactccgggagcgcggggggaggagggggcggtgtgcaagaatgtggatctagacagctctactgtatgcgtggatccgtgtgtgactgcaaggcgccggctctcgggccgcgtttgtgcctggagcagagaaactgcgcgggatgcagaaagccccaagccctgctggagctgggcgcccgctgtcccttccctggcagtcctcttcctcgggcgccgcggggctcctccagggagactgcggaaggggaggaggcgaagcgaaaacggagggcagccgtagtaatagcgcgtagtagttcgggggtatgtggagaaggtgatggaatt

SEQ ID NO: 29 - DNA, 649 bp, Homo sapiens
cagttttgacatcaatctggcgaatccaagtcgaaaataccttcttgcaccagtgtgtttggctcggggaaaaggccagcagaatgccccagcagtccgagcgggcttggctaggcagcaaccctccaggttgtagaagtggacaagacgcaacgcctttccactcggcaaccccccacacagcctgcagtccctggtgcctcaaattgaacccggctggcccaaggcgcccctacgagggcccatccatcccgagttgtgcgtgcaaagcgcggccagctccgcgaaaacttagctgtgtcacgcgagggaggagggaaattatccccgaaaggggaaaggtaattccagggtgcacatttcaccccctccacggcaaaagtcacccaggaggctgacatcctcccctagtctccccttcaaacccgtctccaggctgttcggggagttgccttttgaagttcaatttatctttgaaacattcaataaaaaatgatgaggcactgtcagtcttttggtctcccgacccccagcctcgcctccgaggtgtgtgtctgttggggggcgggggcggcacgggaaggttcgagggttagtccttagcccttttcttgccctgggggccatgacgtgaagacccagctggagc

SEQ ID NO: 30 - DNA, 1319 bp, Homo sapiens
tagaaacaggcggattcaaacagactctacaacgtagtaaccgtgtgacaaggaacaatactgagtcagtttccccctcggaaaacagaggaaagtgaggagtaagaaagcacagagaacaggcgtttgggaaagcgctctgcccaggtgaataggtcacgtgattttgttttggggtaaggaccagaagcggagatggacttccaagagaacgagagcgggaacgtgggtctggttcgaggcactcacccttcagaagttgtagctccactgtatcccgcaggtgcttccattttagccccgggggcttatagaccgcgaaaagcccatgcagccgcgacaagccagcagaccccatacttgaagatcacagcacccgctggacctggacggaagtaccgccaggccccgcccccaaatgtggtcctttccacgggcgccgccatgttccacagccggaagaggttcgcattttatagtcttcggggaaaaccggctgtggagaaggaaatagggcccggcgctgagtgagcgtggttgcgtgtcctttgcagacactttctggggcgaggtgacatggcgagagtcttggatcggtggacgtagacggtagacagttcgcgtgcgtttccttcgcctacttggcctacatgccttctgcccgtgaagcgatgtttcccctcgaaaggccgtaggctacgccgtcagaatcggtttttcagtgagttttgacccctccgacgctccgtcgcctgacagaatcgcggcgttcttcgtacccgcccatcctccgcggacgcccgctgccatggcgactctgctgcgccctgtcctccgtcggctctgcgggctcccgggcctacagcggcctgcggcaggcaagtggcgccgggttctgggcgcaggcgggaaggagcctgagggcgcccggctcctctgacctcggcctttttcttgccccgcagaaatgcccctccgggctaggagcgacggcgccggcccgctatacttcgcaccacctccccacctccccgctgcagaaagcgctgttggccgccggctccgcggcgatggcgctctataacccctaccgccacggtaaggccgcccgcgcctcgcccccgtgggggcggcttggagccgtttcctgtgggtaactggaacatagcctaggtcggggtacccaaacctggttgcagctcgtaaccactcggaacgtttatgaaaatgcagattcctggatcccacctaaacgcactgaatctgaatctgaatctgcagtggagggagaaatgggcgtgaaaccgaaagtctgtctt

SEQ ID NO: 31 - DNA, 375 bp, Homo sapiens
ataactcgtggccggggtcytatgggggaagggcaggsaakykgaygctyattgttttgcaactccagtgtcgggggagakggctttscgcaggatcccccactaacccccgtatggactccctcmwgmcscggtctggyacakacgtgctcgccccgcggcgcascctgrtgycgagggcgsagcgagccgggaagcgcggccgagagggcgagggtggcgggarcgcgcgakgcggcatcccggggaaggggagcccastgtggggytgcacgcamgggctggggattacagggstktcckggtaaaagtstgagagccaaataggtgccgtgacrtcrwtgaaacgrtmmtastacgggcttmtgkktttkt

SEQ ID NO: 32 - DNA, 1178 bp, Homo sapiens
  misc_feature (1018)..(1018): n is a, c, g, or t
taaaaaaattgggaatttgccctgcatttccttagagttgctgtgaaggccaaacgagatggtggtgggcaaaagagctctgccaaccacgcaggcccctgcaaatgtgaagggcctctgacctggagggttgggtgctggccgacctcgtcaatcctccctactccgacccccgcgcggggctccactgaccccgcccctgctgggcactgacagccgcagcaccgaaccgagcggtgggggttggtagcaccccgcccaccctggagcaactgggatccgaaaggatggggtgcgggggaggggagttcgggaccaacgggagttggggagcctgaagggtggtcgctgagccagggcaactgacgattccggagggggtggggcgccaggcctcagcccctctcccggagccctccagcctcctcagcagcggaagttccggtgtcggtaagcggaggtcagaggtcagcaggaagtcgatacgtggctgccgtctgtccccgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaaggtgagccgggccagccccacccccgccgcttctgcccggccccgggagcccggcgtgggctgtggtccccagggtgactggaccagacttgccctcacccactcatactgggctgtggtgactcaagcggggttcctccttgggggtaacccgaagtttgtcactacccgcttggccactaggtcacacccctgacgggcctncaaccttggtttagcagtcatgtcccncctcccagaggttaccgggacattttacctgatttagcttctgggatttccagganccccggtggaagaatgancccactttactncccaaggatcccatttctgggattncctnttattattattacntttt

SEQ ID NO: 33 - DNA, 413 bp, Homo sapiens
agctgcagcctaaacacacagcctccccacctcatgatgatgaagaggcagctgggaacctgtctacgacagttgacatgaattattatgggaaaataaaatctgttatcaggaaaactaatttagaaacagatgttgggggtacagcccaagttagaaatgaattgcatttatttataaatgattctggtctttagccatttagtattttctttagtgatgatacagatctggatttattcatgtgatagtaaccagaagcatcttcttcctgatgttactattttatggccttcaaaaaagtatttgccttcgttgggcatagtcagagttcctctatcaatacaaattctgatgataacaaaagaaaaaaaagaatatattcacatcattttgattcgttgctgaatttt

SEQ ID NO: 34 - DNA, 376 bp, Homo sapiens
acgagcaccgatttcctctacttttgtcgaagaagtttattgtgggtcagggacgtcaggtcgcttgccttcgtttactgtggtcatgattgagcatatgaggacggccattattgttgggggcaaatggaaatgctctaggcggggccatttttcttaggggcaagctgtcgtcacccttgtcaactggttcggatgaagcccctgtggccgccatcttgatctcgggcggccccgataagggaggcggagtgtgcggagaggaggcggggcaactgcgcggacgtgacgcaaggcgccgccatgtcttttgagggcggtgacggcgccgggccggccatgctggctacgggcacgtgagtggaggcggcgttgt

SEQ ID NO: 35 - DNA, 1415 bp, Homo sapiens
  misc_feature (36)..(36): n is a, c, g, or t
taaatgggcccacactaaagttagagaaccacaggntcgctcacaaccctganttctccatgtcagttccgatctttgcgaaccgcagacagggcaaggtcttctctcaggggtcatgcccgcggccgtcctccacggngaggtccgcactcgcgcagccggccccgnggtcgcctcacctggtcgcacactaccacgtcgaactcctcgtcggcgaggaacagcacgtagagcgccaggaaaaccatgcgcacgtaggcgcagacggcggcgccgcggccgccccagcccaggcctcgcggcagccagtccccggcacagcgcaccggtagctcgcggctctcggcgaaacagtggcccgggtcgtagtgcgctgtccagatcttcacgctacacccgcgcgcctgcagcgccagcgccgcgtccaacaccagccgctcagcgccgcccacgcccaggtctgggtggaggaacagcaccgacggcttgggaaccgagtcccgttcccggccctgctcctccgccatggccctggagccgcaactgcaccccgcaccctgatgggggtcttctgcgcaagctccgcgctcgtagctcccagctggccactgcgggccgaccccgccctgccgtacgtgcgtcagttaggccacatcagcgcaaatctgtgagggtctagtaactgcctgagaaaatatcttgtctgaccccggttatatttttccttcggtagggattggactttctgaaggacgttgtgatccaaaggaaggaggccggaggtctctacttcccatacagcaggtaactaagttgtctgtagcagactgtctacaggcatatcgtgagacgacccaggcgtccctggggtcagagaggaccttgcctgcaagtccgggggcggggcctgagtcagtctcgccagctgccggtctttcgggggctccgtaactttctatccgtccgcgtcagcgccttgccaccctcatctccaatatggtatggcggcccttccatgatccccgcctctcccagaagccctgactcctcctgctttgcgccgtgcttttcctctgtagctcccttgcttcccccagcctcgggtgtgggtgtctaggccggggttctggggcaggcctgccgcgctcacccgtctgtctgcttgtctccctctacagcctggtccgacccccagtggcactaacgtgggatcctcagggcgctctcccagcaaagacagtgtccgcccggncggcgggatccactgtccggcagaggtaaggaaccctgcagttcgttcgcttccagactcggagataggacccagaacctcgctgattctggggtggagaccctagcatgtgnagattgacaaagaccaaatgancttctagtgacgtgaccgtgggagtagn

SEQ ID NO: 36 - DNA, 432 bp, Homo sapiens
gcgacataaagagacggccactttgtcgcgggacgtcccgtggtacaattggcctgtgggagtaccattgatcccgccctcatctgaggtgacgggaaagggccgggaaatttccacttctgaataaaacgactgctgaagtgatgacccgcaagaattggtcaaatttggagtacacttcaccctgcgcttggagaaacttctatgtcaaggatgtaagaggcgccgaagtaactcaactcccacctgtcgctactccggtgggcgcctattttatggccggagaatagctgcaccgggattggagaaaggaagccatcatccttaggaaggctccaccttcctgccattgcctggttggaggagcgtatcgtcagtcactatagcagtcactacctctggtacaggcgaagatgtgtaacctatttgccg

SEQ ID NO: 37 - DNA, 388 bp, Homo sapiens
atactccctaagaaagggaataaccttcaagctggcgagagcaatggttcacataaagaaaggcgagctgacccaggaggagaaggagctactggaagtcatcgggaaaggtacgggtgctgggctgcgacgcggccgcggccagcctggccggggggcgtggcggagcccggggggcgctcctggcctgggtgacctttggctgcggttctgcaaggtttgcagcctggaaccgggaaagagcgtttctccctttggtctgggaaaggttgcaggggcggagaggtggggaggcagaagcggtgctgggacaagaggggtagttacagaggctcggcctctaaatccgcttttggaaacgtacggatttccacgccccgccwtaggt

SEQ ID NO: 38 - DNA, 1312 bp, Homo sapiens
aaacaggagtgcctgcccagctgtgtttagacagaaagtttttgataaaggaaaagagtttttgggtcacatttttccactaggaagactgaaaagtccgtagcactcaagcccaggcagatgttacagtgatgggaaaagaagggctttggggacctgcccacttcaaaaacagtttttctcatcctttgccttcggctcctaacccagcttggtgttttcctaagggagctacaaattctggagcatcccatgaggatgatttgctggcctcggtcattagggggaaaggatgttctcagaaaaacagccctgcacgctggtcagcagagatcttgaggtcgtggccacgactgrmcttggtgcagagctgamcccgggactccagctgctcgctgagttcgtccagagccccggtgcatgactccaggctctcggccagtttctgaatcttggccttcagcacggcctggctaaccttggtgtccccctccgcccgcctggggatgaggaagccacgtgagccaaagaagacgatgaagtagacagaattgtacagggcgatggaggcgttcctcccgcactgcagcagctggcggtccccgcagccctgccagcggcagaaaaactcgaggcagctcaggatctctcgcatctggtcctggcaggtggccgcgatctcctgcaccctccgcagctcccgggagttgcagaagatcagcgagagatcggacgtgatggtgacggcccctccggctgtggccacccccagccccacggccgacaccagcagcgaggtccccmrggtgaccgggctgagcgagagccccacgatggcggcgagggcgcccgttgcgctcagcgagctgccggccacgttggctacgagggagcgcctgcgcaggcgctccaggcgccgggccacctcgcgcaggcgcagcacctggccgtgcagccggcctcggcggtccagcagcagtccctggaagcgccgcagcgcgtcgggcccatgcggctcccgggccgccggcctctccattccctgcggggacatagacacgcggtcagcctggagtctccgtgccagccgccgctcccgccctgctgcccggacgccagcgggaacttggcacgagttcagcggagcagccactgccacgctaggggaacgggtctgtttccagtggatcatgagacgagttctcaaagacctgttctggaatccggggggagccacacaagttctcaggaccttatctctgcatatggaaataggaataatgctatcttctgcttctctcccagggaacgt

SEQ ID NO: 39 - DNA, 899 bp, Homo sapiens
attctgtcaacaactgactaggtgacttggtgaaatcacgtcctcaacggtgaaactgataggacagaaattcaaatttccaaactcccgggttggctttcaattccacggtgcaatggttccgcctcccgcgggttcgccgagggagtcattttggccctctcggctcaccgtccgcgcagcctcctgaagcagccgtaaactccgccccttgcgcgcaggacggcgcgaaaacccaattgacaagaattccctccgaagctctgtggtccgatctgcggtccgcttgttttccctgcccggtcccgagcgctcagcctgaagcgccgctttcgagggcaccctgcatacactggccgcgcctcagggatctcattgcccgcgctttctcattgcctctttccgtgttcgattcggctgatctgggcccagcctccgctcccgctctctgtcggtgggcgcgggggaatccgaaacggctcagcagaatcccagcagcttgctgctactggagcgggccgcctccatggcctccaggcaggccgggctggaccgcgtgaggtcctaggagacgggattccgggaagcggggagtatgggtgggggtcctggccatggcggctgcagctgctccgcctcccgtgaaggacagcgagattgaggtgaggttgagtcgaggatctgttgagttccttcctctatcttttgggggattggaaggtgggtcttggcggaaggtgatcctgactttgtaaggggaaaggtgggacattgtgaggacccacagctgtgacacattgtgtgcaacagttgccagagtgtggcgtttagagtcgtgtcaagtcctgataaacatttggattctgagatatttgcggggtcacagtaaattggggact

SEQ ID NO: 40 - DNA, 895 bp, Homo sapiens
taaggcctacgtgcaaatcttcttgacgctgataacgggatgcgagagagccaggtaggttacctattgcagcaggtcaagtgatgatgtcgaaaaggatgcgtgtgccggacactgtggaaggtaaagcgctccgcacagcccgcctaagtgcgcccgggacagagccatcccccaggtgctaccaaacaaatcctccagctgctggtacggggaaggtgttcagtgacacaccggaagcagcctagtggctgaggcgtccttctccctaccaacgggttgcgaaggaagtagttgctgtagccgcgccctgtgcgcctgcgcggaggcgtcggctgctgacgtgtagctggggccagacgggactagccgggcgcgcggctgagtgctgcagaatcgctggggtggcagagccgccagcgaggctggggatgggggcgccgctgctctctcccggctggggagccggggctgccggccggcgctggtggatgctgctggcgcccctgctgccggcgctgctgctggtgcggcccgcgggggccctggtggaggggctctactgcggcacgcgggactgctacgaggtgctgggcgtgagccgctcggcgggcaaggcggagatcgcgcgggcctaccgccagctggcccggcgctaccaccctgaccgctaccggccccagcccggagacgagggccccgggcggacgccgcagagcgccgaggaggctttcctgctggtggcaaccgcctacgagacactcaaggtgaggcctgcgggcgtggaggggcttcgaagactggccgcgggaagcccacggcgccttccgaccccggtccgcggagcgtgggcctctgtgaccccgaaactgagcacagccaccaccgcgacctt

SEQ ID NO: 41 - DNA, 1145 bp, Homo sapiens
ctttacagcgcacctgccttcagattcctactttgcgacacctttttggccggacttggcttgatctgggctccaggatccggtcccaccacccgggctcggagcggcttgttcctagtggatcagggcggctgtgttgccggagtcgccttctattggctacactcccggggactggctgggctttccgacacctgattgggcggaacagccctctgtacgccgacatcattggagggcgctggagccagcgggcggagcgggttccccaggattcttgaccgggcgcgctagtccgtccgctgagccgggcgcggggcgcaagagcggagctgcgccagccgctgcggacggaagggctcctagccaattggggtctttgaggcgaatgcgaggctggggccggttgcctaccggccgcttctcgccgaggcagtccagacttttcccccggcggtgcccgctccaagacagcatcwgtcaacgctcctcttctcccctcctcstcctgccgggccggsctccgccggctgcggcmgagaggacgcgggacccggcrcgstgagcccatcagctgtcaggcgagcggcsaakcggctggagggcggcsagagacacacaaagaactcggtgggcggcggcggcgaaaggagacggcaactcctccccgcgcccgccggtgccaccgccggccgtgcttgttccgaggccgcgcagacaatgcggccgggctcgtccccgcgtgccccagagtgcggagcgcccgcgctcccccgaccccaacttgacaatctcccggctcgcccagccccctcccggggtaggggcgccccctccctccggtggccggcgaaggaagtcggtacgcggccgcagatcccggcaacttgcgaaccgggaaaagtgtgcggcgcctacgcggggcggcgcgacgcggcccgcccctcgcgtccgcggtcatcgcgggtgactttctcgactcgtcgtcagccggggccgcagcgcggccggtggggactgcggggcgggccggagtccgtccgagggctcccgcacctcgggctgcgggtgagtcggctttaggcgccgtgggctcaagtggtttagggcacctgggcttgggaagaagtcgccgaaaaaggctt

SEQ ID NO: 42 - DNA, 686 bp, Homo sapiens
aaaaaacgggattttggagaagactgaatctaaactaatagaaatctgaaataccaaagcctatttccttcccactctctgctcttctggggaactctggtgccgacataaaaggcagttcgatagggcacgcagcagctctttgagcccctggcatcttgatgcaggtccagctcagtcacaaaatggctgttcctttgtgccgcagcgctgacagacatactgcattgcactgtgtactcgccaaacagcaggaagttgccgtgccgagttcaaaggccggccggcgccggcgcggggtgctgattggctgtgcgcgcgcctggtaacagcagggtcaacggcggcgccttcggcagcctccgccccgtgacgtcagacggctccccgggggggcggggagagaacgcagtgacgtctggccgcgtgcgcatgtcgggcgctttctcctccccctacccagggagccgcacgccgaggggaaaaggaagggaactctagttgggactttccggtgggcggcttctttcctggacgcgtttgctcctggcagtcttgtccgcccttgcctgtagcggtccgcaccctgtagtgtgttcttttgcccatttccgggaatggtttatcctctttgagaaacggctgcttttggagaaaaagtgcacgcactatttataaattttata

SEQ ID NO: 43 - DNA, 314 bp, Homo sapiens
twaggtgccgcgaccgccgccgtcgtggggctctccacagagcacggggtctacgccgtcagcagccccgccccgtcccgcccgggcgcccccaccgcgcgccgggtgctaaagagacccgcagggggcgtgagcttccagcgccagcggcttcgggtctcccgtgcgacggcagtgtgagaaagaaacgttccgggcgccggtgctcctttatgtccggtcagctcccgtctggacagacgctcgaggccgcctggaggccgcagtgcggctcggaggccccaccggccgcccccgcagggctgcrrmgcctt

SEQ ID NO: 44 - DNA, 1404 bp, Homo sapiens
ctatttgttccctacggagaaatactggttttgtgtttgaggttcgaatttgtagagcaataggggcactaataacgttgggcaagcagaccggtgggccctttgaagtctgaagaatcaacgcgcaaaagggaggaggtgagcaggtggtcgttcaaacgtcaggcgagggcaccagctggatctggaggaactgagacagtgaaaggaaagcaggcaaggttctagtctgggatatgcacgaatcgcggtctcccttccctctctatgcctccgcagtccttcccgttcccaaaggaggaacagcccgagggctcctggagcaacggaaggaggactggatgcggggaagggcaaagggcgcggaacggggtggggagtctgcgggcgagctcggaggacactggcgcacctcccccgccggctcacggccgcatccgccacatccgaaggcgccgcgacttcccggagaccccgaagcgtgcgctctcatccgggtcctgcgggcgctggcggaaagaggcgaggcggtgcctccgggggcggggcctcctttcggttggcggcctcgggcttcgggagtcctccaagaggccaggtgaggccgtcccgtgatgccccgcgccccggccgctctggcctgcaacgtgtctctggggcggaggcagcggcagtggagttcgctgcgcgctgttgggggccacctgtcttttcgcttgtgtccctctttctagtgtcgcgctcgagtcccgacgggccgctccaagcctcgacatgtcgtacaactacgtggtaacggcccagaagcccaccgccgtgaacggctgcgtgaccggtgaggctgccggggccggagaccccggcgagtgagggagttggggggagccgcggcctagggaggcccgcggccccgggggcggccgggcaggagtgggcccggcgaatggaaggcgctggcggccgaggaacctcccccagcaccgcgacctcgaaagagtcgtccctgtacgcgctttttgggggttggaagccgctctcgagggtcccctttcgtcctggggaggtcattcggcctcgagaacagggcgggcccagtgagacagaggagtgtgagttggttgtgcggtgtcgtttgggcctggagattggctttctgtggccaggaaaaggggaggtggtaatgacgactttggggtcaagattttcctgtggggaagtctggggggcccaggaggtggagggatgtgaggaatgcccgcattatcccacagagggagaggagccctcttcaggctaacccctttgcctggggctgttgtttacatgagggccctgctggagaagagggtttggtttgcctggcccagtgctgggtttgccttgttgacgatgcgcatggattgt

SEQ ID NO: 45 - DNA, 1643 bp, Homo sapiens
cattaggtcagaagtttgagaccaacctggccaacatagtgaaaacccttctctactaaaatacaaaaattagccgggtgtggtgtcacacacctgtgatcccagctactctggattctgaggcaggagaattgcttgaacccgggaagcggaggttgcagtgagccgggatcacgccactgcactccagcctgggcaacgagagtgaaactccgtctcaaaagaaagaaagaaaggaaagaaggaaggaagggagggagggaaggaaggaaggaaggaagggaggaaggaaggaagggaaggaaggaaggaaggaaggaaggaaggaaaggaaaggaaggaaaggagaaggatttgtgcagaagggaaggaaggagaaggatttgttcttagagggaaaagcatctaggactagactcaaagacacacgtgtattcatgtacacacctaacttcagaagggagggactgtgctacatacacctcgagtaaacatatggagatatgcaagcacatgcactggcagagactacagatgcaggcacatacctggagggactctgccagaatgcacacaaacacctacaaggtgcttacaggagctcacacacaggatccagaggcacgcacacaacccaaagggtgatcctcagggacatgggcacatacacacctagagatcattcacatacaagcgttgcagatgcatgcacatacctagcgtgcactcacagggtgtacgtggacacacacacccaggagttcacacaggcacacacctagggggcgctcccagggcccaagcgcacacactcacctcaaaggtgtccacagaggcacacgcccccaggacgccgacgcacgcctagggggcgctcgcagggccggcgcgcacccgcacgcgcacacgcccctgcccagcctcctgcccgcctccgccttcggaggctgacgcgcccgggcgccgttccaggccttgcagggcggatcggcagccgcctggcggcgatccagggcggtgcggggcctgggcgggagccgggaggcgcggccggcatggaggcgctgctgctgggcgcggggttgctgctgggcgcttacgtgcttgtctactacaacctggtgaaggccccgccgtgcggcggcatgggcaacctgcggggccgcacggccgtggtcacgggtgagtgcggaggcgggtgagtgcgagctggcggggcgcgcggagaggaggccgggccggcggtagcagcggcccgccgggctcagctcagctcggctcccgcccgcggtccgcaggcgccaacagcggcatcggaaagatgacggcgctggagctggcgcgccggggagcgcgcgtggtgctggcctgccgcagccaggagcgcggggaggcggctgccttcgacctccgccaggtgagaggcagcgggggcgcgtgggacggtgagccgagaacccgccgggacacagagaagccagcaaaggggctgcggaggatccgggtccagggaggagagcgcggcgcgggcggctgagcgcgaagagggagtgcacggagcgctggggtcacccgaggaaggcgcaccagagccgagtgggggaggggcttcctgaaggccgtaattgcagttctgaaacgtt

SEQ ID NO: 46 - DNA, 1640 bp, Homo sapiens
  misc_feature (919)..(919): n is a, c, g, or t
aaaatacgtttagaaggatctttattttttcctgctgctgtacgaggcaccctttctggccccgtgtttccttgcccttctttccagcagcagattttactcagaacaacttcagtgctgtggacacaaagacgcaggccttggagaggcgggtcagccctgcctcggggctgctgagtgaggtgccagatgcgtttgttcctcatttactcatttgcgtttgttcctcatttactcgttcctcgcagactgggttgtaacaccatgtaccaccctctgaacccagagatcaagggctggctggccctgtagccccaaggagctccgtcaccggccaatggggaggcgaacccgtggtgacccactcagcggcaccgccaagggctgagcgtggcgccagcggcggaaggcccggggtcaccgtcccaggccggggctccgaccccgagtccgcagggtcctctccaggcaccttccatctggggtctggcttccactcccggccgcggcgtcctgatttccagaaaccaggcggccgctggaggggagaagggggagcgggcgcagcgggggagggaggagaagaaacgccggagaagggagagtagagcgaggaaggacgcgaggcgggcggagcgcggggaggtgcaggggggcgggaaggggcggcgccgcgagggcggctcctggcggcgggactgtggctgtggccccgggagagccgggtggggcctcgggatgcagccgccggtgcccgggcccctgggcctgctggaccccgcaggtgagcgcggggctgggggctcgtctcggctcctgcgggggagcgtggggaccccggggctgggacacaggtccccggccggcccgggcggaacctgcgcggagacgcggcacggggtctggtccctccgcctcnttcgagctctgtctttggaacactttgcatttcgcactggggtcgggcgttcgtcctaccggttcgttcattcattctctctccctgtctctctctctgtctctgtctctgtctctctcgctctttctgtctctgcctcctcagtctttgccttcctctgactctgtgtccgttcctcttggtctctctctccctgttacgttttattatgaggaacaggcgccccaaagtgcgctccttccccatctcttccgtttattccaacctacaccattttccgggaaaacttcgtttggaagagcgaggtagccttttcctgtttgaacgtgcagaggcgctcacagaactggacagtgttgcttggtttcggctcgcccctctctcgtgtttctccccggtgtgttcacggaatctccaattcttccacttcccagaccaagcccgctgcagcctcggagccaggcaaaggctgggaaaacaggaaacctgttgcgtgttcaccagttcgcaagccgggctcgggggctctgccgggaagtggccaggactggaaggatgccgggtgtcttcctgagggggagagggctgggcagatttcagaacaaaagagaaacagagttccctagaaagtgaagccctgacggctggagggagggatggagtggtgtctagggggctctgccgctccctcagaaataatccaacacaaacttctggggat

SEQ ID NO: 47 - DNA, 908 bp, Homo sapiens
attgtcaacactcctgccccctgcattttggccgaggggggaaggagggaagaccaaagaaaacaaaccaaaaaataaataaataaagcgagccagagccgagatctacaaagttttgcaccaacacttacgcagatccgtttgcaaagtcctaggaaaccattttcagcagatgtggggggggggaggcgtcgacgtcataatccccggaacgtaaacattgttgccgatcgcgctcggagcccgcggccgggctatttgggggggctctcggcccgggcttgggggggtgggggcggcggcccgaggaggtatggggcggggagggagcccgggaggcaccgggcgccgttagcgccgtggcgtcgctgatttgttgctgggtcacgagtgcgcctctgcacgcagacgccgccgccgcccggaagttgtgtgcggagctgacttttggagggctcgagttgcatgcggcgggcgcatcccggagatggcgggcagggagggagggaaggaggagggggcggggagggaggtgcagatttgcacggctggcctcagcgggaggcgcggaaatgcaagccgagcatgctggcgtgaaagcccccggggcctggcacccccaaaaacgaccccccacctccgttcctgccctcattgcccacgggtaaccctcttcctcttctttcatccacccctcccccaacattacctgtgtggccgactcaaattcaaagcattttgtatatataactgttttctttttttgcttttctgctgcatgtctttttcgtggggggtgatcctctgcagatccccctcatttgcggacccgccatgtggaggaggcatttggggggctcagaaggaaattagctcggcttccatacctataaggactgcctaacttt

SEQ ID NO: 48 - DNA, 521 bp, Homo sapiens
aatgctaaacgggtgagagtgtgaaggggggagcgtgcggtaagtgaaatccatgaaccatttctctggactgaagaatactaaaaacccctggattcacgttccaaatttccaaagagctacaagatgaggcaccagctggggctgagggttctcacccactcctgggggaatttgcctctcagcacagctgcccctctaacgtgactagtgatctgccagcgctcggtgctcatccccgggatcttgaacttcgcctccctcttactttctgccagacccgcgaggtcaggaagggctctgggaggaagcgggagcgcagcggcgaggcatggaagaagagggttgggggcgcgcgttacccgcaaaggttctcgaggtctccttccagccctggtggtaatcccgcagcgccgcgccctcccccagatccaaacttttgctgggacctccgggtggcgagcggcggcgacggcggcggctgctggagccaagccctgtcgcccgcagcgtcggggcgc

SEQ ID NO: 49 - DNA, 409 bp, Homo sapiens
cttacctctgccgctgcagcagcgagcgtcgacctcgcggcccaggccgccgccgccaccccgaccccggcaccagctcccagccccgacctgcccctcgggagcgagccccgtgaccgcgcgcgcgcgaccccgccggggagccggccttgcggaacgctcaggcgcgctacttcagagcatgcgcagcaagaccgcgcggcccgttccttaggtaaaccgctgctccgggtcttccctgcgcggcgccgagccgcaggcctggcctgtcagcgcatgggggcggggacgtaactcacggaaaaggcggggccatcccgagattggacggcggcgtccaggggcggagctaggatgggggagctgaacaggacgctcttggcgtaaaggggctggagtagrrrgsgct

SEQ ID NO: 50 - DNA, 1973 bp, Homo sapiens
aaaacacttggcagcctgcttcagcccaagctgaggccacccctagcctctgctaaagccccccactcccaatggtccccgccaaccggataagagcgcgcgcgggacccgccttcccctctcggcaccgcccccgcccccgacccctcggatccccccccgcgtggctcctcccttttccgctcctctcaacctgactccaggagctggggtcaaattgctggagcacgctgatttgcatagacccatggccaagctgcatgcaaatgaggcggaaggtggttggctgagggttggcaggataaccccggagagcggggccctttgtcctccagtggctggtaggcagtggctgggaggcagcggcccaattagtgtcgtgcggcccgtggcgaggcgaggtccggggagcgagcgagcaagcaaggcgggaggggtggccggagctgcggcggctggcacaggaggaggagcccgggcgggcgaggggcggccggagagcgccagggcctgagctgccggagcggcgcctgtgagtgagtgcagaaagcaggcgcccgcgcgctagccgtggcaggagcagcccgcacgccgcgctctctccctgggcgacctgcagtttgcaatatgactttggaggaattctcggctggagagcagaagaccgaaaggtgagtcggcctgcggactcttccggcccgaacttctcttacctaccccgcgctccccggtgcagccgggctgtggaaggcttgcaggggaggaagctaaaaagtttgcacagggcaactcccgcccttgctccctcgggactctccgtggagctcccacggactgaaagagcgtgccccccaacccgaacgagccccgccggggcctttgcaaagggcagcagtggccgtcgctgcccgtgcggctcccgtggctggcagcctgtggcaggggcactctcgggacttctcacgggacgcccggtccttgggcgtgcaggggtcatggggggtgacggggccgcgggagcgccgggttttcgtagagcccaggtgcgcggtggtgcttgcattcgagagggaggggcgtggtaccggacgaggggggcggcgatggccccgagggcaccggggctgacgggacccctcgcccttgcccgcgtgtaggatggataaggtgggggatgccctggaggaagtgctcagcaaagccctgagtcagcgcacgatcactgtcggggtgtacgaagcggccaagctgctcaacgtgtaagtggggcccttgcgcgtcccccatggcaccccttcccgccccagcccgggaggtcgccttggctgggcgcccctcgcccggccgcgccacttcctgtcgcttttctgcctgtctcggaagggagggggcgagcgggccgggcggcgacccccagggacccgggcagtggttgagggcgcccgcgcttctgcgctcactggccccgcccgctgcccccagcgaccccgataacgtggtgttgtgcctgctggcggcggacgaggacgacgacagagatgtggctctgcagatccacttcaccctgatccaggcgttttgctgcgagaacgacatcaacatcctgcgcgtcagcaacccgggccggctggcggagctcctgctcttggagaccgacgctggccccgcggcgagcgagggcgccgagcagcccccggacctgcactgcgtgctggtgacggtaagggactgggggactgcagcctgcgggggagagccccggaaggacgggagtcagggctgggttgcatgagtgtggatgtgtggtaggtgggggtcaggagggtggctgccttcgcccgagtagagtgtggctggactttcagacgagatgtgctagtttcatcatcaggattttctgtggtacagaacatgtctaagcataatggggtctgccagcagcggaagagatccctgtgagtcagcagtcatcc

SEQ ID NO: 51 - DNA, 1095 bp, Homo sapiens
aacaatagattacatggtaaaagcaagaggctaactagacgaaacttctattgagcagagacagtgtcttacatgtttttacgctcagagctttatataatggtcgataaatgtttgttgactgtacaaacaaatgaaggtggccaatggagaagaaatgagggtcagaaaaacatccggaaaaggtgggtaactaagcagaggataaaggacgaggtgtcagtgcaaaaacgaaaccttatactttcaagcacaggtattaccttatactgatactttgaagcagcaaatttctccacacccggattcaggatttgacatctaacagcaaagcctgcactgacgtgtcctcagtatccagtattggcgcgtcgcacaccgcgcgcccagaaccacacaggcttccccagtccaggatcagttggcgcagcccggaagcttcgaggagctcctagaaccgccgccttagctggcggcgccggctcttagcctggaggtcggcggaccgcttccaaaagtggaccgaaattccggggatggatgccgggtggggacgcggacggccgcaggccagtctcctgacagccggggctgagggaaccggccgcagcaaatcgcacaccctctctgggtgccagagtgactggcgcaggacgcgtaagagaggagcgagaccccggaccagcccgcaccgcggaggaccggagagggcgattcggtaaagggaggcggacgggtgtcggggcgggaggcggtgactcacctgggaggtaatcatgatgctggagtcgatgaggaaactcatggcgaagtgtaaggaaggctcctgcctccacttcccactccccgaggccacagcacgctggggccacaggccccctcacacggtctgccttctcccaggtgctgcagccgggactgcggctctccagcccgacgccatcaagctgcgccccagtcagccaatcagcgtctacatccacagccccgcctcgggctcggggtgggtggggcggacgctaggctcccgccgcggaggccccaggggcggggcttattaggggcggtgctaaaagcagcaatgcaaggtagcgt

SEQ ID NO: 52 - DNA, 576 bp, Homo sapiens
  misc_feature (201)..(201): n is a, c, g, or t
taaaatattttccccccaagtctcaaatattgaagaatctctaaccaggtacagtatcactctgctctcttttctggttgaaaagaaagaaagaaagaaaacgcaggtattgcttttcgaagtgctttgtaggtaatcgaggtaagaaaatatacgcaggcagcctcttacaattttatgcaggggcgccgggagcctcgntgacgggcggtgggggcccccacaaaggcgcggctgggagctggcgctctttgccccagcagggacgaggagcggctggagaagggtgtttctttgacccgtttacaggctcgaaacgccggtctcagtggctattgagttacccaccaagttacccccggttagagctgtttggggtggctcacgttattgcgccaaactttggagatgacggctgagtcctccgggcgaacatgtcaagccgattgtaaatgctgttatttccacatccttcagggacaccagtccctacgaagaccttgggcgattttgaagtgcgggcacctcgattaccccgaatctgtagtgtggctggtatcggtgttncccctggtt

SEQ ID NO: 53 - DNA, 310 bp, Homo sapiens
aaaatgcaatgctgggagatcaaagtacaatatttattttagaaatttgttgtaatcaccaagagatacaggtatcaaggccaaatgtcagttccattacacaaagccccgcgcccgggaagagcgtgtggctgctctgccataatgtaaagagacatcaaagaccgagcgcgctgcaaaagtgcgcagctgtgggggagagagtaagcccgggagggccaggcgcgccccagacagcgcgggggctgcgggggcgggggtcagttcaaagaggtatccgttggggcgcgagttcacaggagggaagatt

SEQ ID NO: 54 - DNA, 829 bp, Homo sapiens
aaagtttaggcaaagattttggccaattccaactcaagtatccggatctccagcactacttccctctagcaatgccatacacatctaaccctaaaacgaggaagcgtccgctgaggttgcccactgcaggatgctggtgtgtcgccgggcacatgaaagtccctgagtcaggggagagcgctcgccggggactgcgggcgggagccggcgactgagaaccgcttgccccgctctctggcgtgagtacgcaggcctcctcccacggtctcaggaagcccagacgccgcaggcttccccgccgtagaggagctgccggggcgtaattcctccaccgcttcctcctccagctgcacccacccgtcctttcctgctcgggagggctgggtttgaagcgcgcgccacggccagcccgggaccgcgggggagggcgagggaggcgcgcagccgcacgcacgcagtaggcagccccgccccgcccctcgaggcccaaggtcccgcccctcgaggctccgtgccccgccccccgggtgccccgcccctttgcgcggctggcgcggccagcaggccaggctcccctcggcaaacctgtctaattggggcggggagcggagcttcctcctctgagggccgtgccgcgctgccagatttgttcttccgcccctgcctccgcggctcggaggcgagcggaaggtgccccggggccgaggcccgtgacggggcgggcgggagccccggcagtccggggtcgccggcgagggccatgtcgctgttgggggacccgctacaggccctgccgccctcggccgcccccacggggccgctgctcgcc

SEQ ID NO: 55 - DNA, 142 bp, Homo sapiens
  misc_feature (71)..(71): n is a, c, g, or t
taaagaaacagtaccgggggcgggccgagcgacgcagccgggacggtagctgcggtgcggaccggaggagncatcttgtctcgtcgccggggagtcaggcccctaaatcgaagaagtcctggcgngccctcccccccctccc

SEQ ID NO: 56 - DNA, 317 bp, Homo sapiens
  misc_feature (49)..(49): n is a, c, g, or t
aaggtcttctctttgttcacttataaagtgaggaaaacaaattctcggnactggcgtgagagttgagcgtcacaaaagaaagcaaaagaaaatattagtgccattattgtggcgaatttcatgtttcccagcgagccctttgattcctggtttgggctggcgctcgagctctccagccgggtatgacttcggccacaagatggcactgacctgcaaacaaagaaaagcacagtggcaccgactttttcaagcctcgggaaactgccctgccttccccggagtcgaggactgtggggattagggcttcctttcccctg

SEQ ID NO: 57 - DNA, 215 bp, Homo sapiens
attttatgaacaactgtagagcttactctgagaaaagaggtttcatactgacctgtaattggatattctctttctggcctagtatataacctccctgattcaggctgcggctcagagttcttcaaagagtaagtacagttatcactgttgaaagtgactctggctgtaaacatcaaagacgtgatttctaatgttgttgtggctgaaactccgct

SEQ ID NO: 58 - DNA, 242 bp, Homo sapiens
gaagggcaagtcacccgcggttactaaccctcccccaccgacatcaagggagaaacactcctctactcttttctggacattcctgttcagagtcgaggtaagggttgcacggccccgcccctccgttaccaagccttctctcgtctgcctcgcaccctcaggccgttacaacccaggaggggaggggccgggaaaggtgaggctgctggcgggaagcgggggcggcgcgaatcggtacctag

SEQ ID NO: 59 - DNA, 769 bp, Homo sapiens
gattcccgccccacctcgcccctcacagaggaaaggacggcgcagataaaccctcgacacgtatttgtagtgtggacgcgccccaccctactcctgctgggtgaarccattcctctagaggtacccgccgcgtaacacaaggcagagggaggcagccctctmttagaggagycgagtggggcggggacggtcgcgccccagtggyckccgccccgagcgtccgcctgccccacccttctcatttacacccaaggaggccgctggctcctctggmtgcggcgcwccctgcgagctggctgccgcccccacgtccgccagttccatggyccaggcctcggcgtcttyctcgtcgtcgtcgccgccgccttcctcctccggctccacccccagcttctccatcycgccgctgccaaccgcagcgctacttccggccgcttccgcctggtctccagggaaaccgggggactacagtctccagtcgctctgtgcctctcgcctgcgcagtcgtggccgaccagcctgttgcccagagtgacacgcatgccarggaatcgtggaggcccacgtgcgggagttttttycckccgggtggaggccggaagcggagagattgaaagcacaaatggctattttttttttttttgagacggagtttcgttcttgttgctcaggctggagtgggcaacagtgacgcaattccggctcactgcaacctcttcctaaaggattcaagcgattctcctgcctcagcctaacaagtaaaagggat

SEQ ID NO: 60 - DNA, 598 bp, Homo sapiens
tacaaagggaccgtgacgtcccttaggagacggagataaacgatggccgagtgcgctgggaaggtgggagagcagactcggcttcaggagaaacccacgcccagcccctgattcccttgagtcacactagccgcgggccacaggcccccccatgaattcttccctcagaattctcgtatcacaccttggcccagctcccacccgagcaccgcgctcttctccctccctgatccaagccccaagcaaggcggcccctgcggcatttatcaaaagagaagcctccaactggtggagcatacaccgacacagccgccatcttcccaagaaagcagttccgggtcggaggccggtgcaaacaaaaagagccgagcacttccggcgtcgctggcaacgggggctgaaggggcggggcaaacgccgcactagaacgattgatcttagagattctggttcctagagaggccagagatgtttgaagtatatcgccccaagtggcacggaggagaaagtgacacccggaaccgggaaaggacttgtccaaggtgaacgcaccagcgaccaggtctcctcccgctcagactggacttgtttctagggt

SEQ ID NO: 61 - DNA, 195 bp, Homo sapiens
aaaaaaaaaaaaaaaagttttgaattcctcamtgatttttcttctggaaaggcrgcttasgataattatttcagctttattgagggcagattagttgaagtctgggcgctgcgwttcratacgcgtctgtacacgggccgacaatgtggtcattgttggctmctgtgtgtgaatccattcaacatatrcactttt

SEQ ID NO: 62 - DNA, 524 bp, Homo sapiens
acccgtccgggcgggcggcccgcacttcctcccagtgtcgcgccccgccgccctggcccgccctgctgaccgcgcccatgagcccggagctgggcgggaatcggctgaagcgccgggcgaggcccgcggaatcggcccaggcggcggcaggtgtagaggagcccgggtggggcgcgcctctctcctcccgccggacaagggcccttcgcaggcagagaaggtggggctgcccgggacatggaccgcatcggtccccctcctccccgccagcgcggtggcttctgtgactgcccaggacccacagacacccagcggccctttccttttgctatgcagtgccggggaagagactccactgcatggcatattccctggagtgtatcttggtcaccaaaccaacaagctccaagctactgtgctggagcactcgggaggcctgggcagtgctgagagggaccctgtaggtgaacaccacacttcacggcgcactagtgggcaatggggcacgctgcttctgaaggctt

SEQ ID NO: 63 - DNA, 1070 bp, Homo sapiens
tatgggtcccctcctcctcctatccgggatcccacggccgggttcagctggcctccggctccgctgcaaacacgggaaacctgcggaactcccgcggcgccgctcaacagccactcgcccggcgccgtcgaacttccgccgcccagtccgccgcagcgggcctgcggcccggacccgggaaccaagggggccgggcagccaatggcagccgggcagctccgggccgccgccgatcaccgagcgccgcacgcaccttcgcggcccgagcgcggcgctggcaaggtgagaggcggggcggcgtcccaggcggcgccctgctgggccagctcagtgaccctgacctggtctccgaagggattctctgggcgtccacgggccggcttctaaagagctgggatccccagcctccctctagctgaagatggagaagaactttttccatttggcaatttgtacctgtgtttgcccaggactttttatttgggtcaaggggaatagttatcccaatactaacttctcagtttattttcagggtaaagagagaaaaaaaagagagagagagagattgtagggatcgttagggattcctgtgatggaccttgggcactaaattccatattgcgttgtggagatgccctttcctcaattgcttttttccacaatcccaaatactgtcaagaaaccgtatcttacatccctaaagtcagcgaagattcttagattgttttctgagcattgggttgttcaaaagtaaagatgatgtgaatcacttcagaaacaggttgtgtcattcctttggaagcagagggttgccctttgtctctgtgtgagatggtgctaggcctatcgcgtgctcaggctttgatgaagcagagtcatcacattggtgcaccaaagctgaattcatttcatgccctaattcaggagcaattgcatctaataccatcagattggtcggtctggaagggagcctaacgattacttagtctagcttcctcactttgcagggaacgaggactccagaggtgcagggacctgcgtgatgtcctagcttgttggggacagaggca

SEQ ID NO: 64 - DNA, 597 bp, Homo sapiens
aacccctctgccccagcctagtccgtcccgcacacacctccccttcccctgtcgcccatttccccctcggccggcagtacggactgcagaaggggggcgtgggcgccaggaggcggcctctcccgcagcggggattgcctggggcggaggacctgcgtcgcggtttgcggggatcgccttcggaggggccgcacgcgctgtgtgcaggcggatgtgaggagcatctcaagaggcgggtgggggaagcgggatcaggttgttactactgcagagagagagagaggaagagaagagagagagggagagactcgagagcgagcgagcgcgggagcgagggccgcagcggcagggccggcggggaagtgggaagagggacctggacttcgggaccccagccgcccccgcccccgccctctccaccagctcaggctgaacgcgcctggaacgtcccagggtaagagggaaccccaggcggggcaccccacgagggcagccagtagtcccgagcgaagccgtgcctggaccgacagtgggcacctccagggtctgaggcgcgcgcggacgcggcgtcaccaaagggggacactccgacttttg

SEQ ID NO: 65 - DNA, 158 bp, Homo sapiens
aacttatgacgtcctagtttgaataatatcaacttagtttcggtaggtcataagagctttgctcctgtatgtccctgcctttccctttatattgtgattatcacatactgcatctttgtacactctatgaccactaacataggtttgcaatcactgtt

SEQ ID NO: 66 - DNA, 1045 bp, Homo sapiens
aagcatttgtaaaaggctattttctcagctgtggtaacaaacactggcagagtcttaggcaaaacacacgccgtttcgcccgagttagccagccactgagatcgttacagttgggaagcagcgagcccagcagcagggatttcacggacccggtatcagggttctcgggtcccaggggccgcgggccgcagagtgggaggaggacccggcagctgtggccacccgaggcccggacggctccgctgtcccactcgccgcgacccggacgctttggcaggaccacgtctggggcgtccccgccggggggccgggtcagagagcctcgccacttgaatcacccgacgaggcggacagctctactcacctcagcgccagcagagccagcgctccggagcgcaagcggcgctggaagccaagcgggggacggcgggcggggcggggcagggagccaggcgagggcggggcggagcgccgggcgtcgggggcggggctccgggggtggagcggaacccgcgccagagggagctggggaagagggagcgggagaggagcacggaacctcgcgataatcccctgtgttctcgcgctttcacgataggcgcgggaaaatgctcgtttgccaagcgatctgtgtattggccgagagcgctacttcctctatgctggccccgcccgcggctctactcttgcttccggctccggcgtggggtttgatgtcgacggagtggcttttgcttagcgtcttgaaggggtaatttagtcggctccaacttcgggaaccgccagttactgacctgttccttcattcatacattcattcagtcactcggcttctcattcagagcacacgttgagggattacaacaatgatcccacgttgagcgttcgttctaagagctgtttatagattatctcgttgaacccttgacagcaatcccatgacacagggactaacagcgccagtctgaccgttgaaaaacaggccagtgggtttatgtaacgaagccaacgttatttagcaagtagtgagtctaagcaaaggatt

SEQ ID NO: 67 - DNA, 289 bp, Homo sapiens
  misc_feature (4)..(4): n is a, c, g, or t
taanttcgncggaaccggactgggttcggaatcatcaggtcgcgacgggcaaggactagcccttccggcgnataggnaatgacgcaactccgccctgcgcggncaaatggataaccggtagcggtcaccatagagatggatgaactaggggcggagacactgggaagactgaaagcttgctcagcggagtcgcgcaccgctcctcctattgggcggcagngaccaatagaatgtggagctcgcccnccgctgatacgtcacgtctgggaccgcgggagtattccaggcg

SEQ ID NO: 68 - DNA, 1284 bp, Homo sapiens
taaaaagtgaacaagagccaaaaaagccacaaacacaacctataccttctaagaagctcgactactcccgttctgcacgttccttcccagaaggatgcagacccccggtgtcggcgggaaccatgaagctcttggggcctccctgggccagacctgcacccgggaagccgccgcaaggtttcaggagccccaggtcgccccaaggccgtgccgcaggattgaacaaagcggggggtgcagcaacctcggcgcttccgagcgtgttcggggccggacgcccggcgtccggagctggctccagaaaggcggcgggtgtgcggcgcataggcctatctcgcgccctagccgcggcccgcgccccgccgcagtccccggcgacgggactcacacactcagagctccaccacgtacgcgtagtacgaccagacgaccacgaatgtgatgaagagcaccggcacccaggccacgacgcgctggcagcagcgccacagcgtccagggcgccatgttccgctggcgggtgccgagccccgcgtcccaccgttctggggagcgcgggagccccggcgacggtgactcgtacgctccaggcggctgctggtccagctcccccgcctccgaggcaggacttgtggtagcaaaagtccgaggcgccgccgggactcctcccgtcccggcgcccagccaggccccgcccccaccccacatcccgtcgcccccctccccccgcgtccgtccccgccagcccgcccgccgcgggccgcggtccgcccccggtctccgcgtcgttggggcggggtttccgcggccggggaggcggtgagggggcggggccgcagctcgggaccgcccatggggcgtggcggggctggatgcggacaggctgggcggacgcgggaggccggggaaggagaggcgaaaaacacgcccttgatggatgagagggacaggggatagcttttgtggatcgagactcaatgggggagggagactatggaaatgattccttagctgatttgtggatcttggccctctctgtgtacagaactcgctccactggggttcctgggacattgagtagttggggcactgtttcaaacatgacaaggccattccccactcagtgtgcgaagaatgcacattgttagaagctcctcctgtgatttttatgttcgtccatcttggcggaacccctccctttcttgaagatggaacggtagatacctgtaatttcaagtagaagccatgtagggtccatcagtttgattcgttaaacaggtttatg

SEQ ID NO: 69 - DNA, 157 bp, Homo sapiens
aagctacgccaaaacccacatgatcagtaacgtcggcatgcgtatttgcatactataccaccacccgagctgccacccggtctcggattctccagaaagttacaaagtgaaattcagccccctccggagacaccggcgaaatggcatttttcaagaa

SEQ ID NO: 70 - DNA, 1613 bp, Homo sapiens
aactttttggaacagttgttctgatttcggtgactatgatgtaaaactggttgacctgtaacatccacctctggccccaaacctttctatcctttccaccgtccgctccccgtccgcccggcatcctcgcccaggctccgccccgctcctaggtcggcctcggtccacgctcggctttgtccaggggtttggccacggacaagccgcgggagcggaggccacctgctcaccgcagggcccggaatccacatccccagcagaagcagcctgcagactccgtcctacccgtgcaccgcgcgcactcacggggaccccaggacacgggcgagcccggaacccgaggacccaccacgaccccgaaaccgggcgtccaaacaggctagcccgcttacccgaggcggctccgccatggtccccgcggtcccgcccggccctggcaaacccgaccgtctccggcggctccgcaggaacctgccctgctgcgcactgactcccctccgctccgccccaacccgactcccacgcacagcccgggtggcccccaaacgcagcgccggccgggcgcggtgcatgctgggacccggcgcccggccgcggctgccgctcagtcctacagcgcctcctgcgggaggaagaagcggcgctgcctgagcgtcggtcctcagaggagggacccaggggcccgggcgccctcagagcaggccgcagcacgccggtctttgagggaccctcggattcccgggatctggggaagggaagcggtcagagacaagctgaggagctggccggaggggaaacggaggggcaggacatggagagatcagggacagggacagggagagatcagggacagggacagggagacgcgctccacagaaacccagactgagaggcagcgagaggggaacctccggtgcggttcctcccaaagccgcccccgagacaggcttcgtctgtgagggggtcccgggaatccagcgaggccggaaagtgggtggggggcgcgcgctgcccgcagtggcccggggcatccgtcccgccgggggcgcggaggccgtgggggccccatgcttgtcccgccggggacgcggaggcggggtccgcccaccaggtccggtcgccgtgggtctagagccctccgggctccccgggcggccacgctccttccttagcagagaacggccttgggcagagaccccatgcggaagccaccccggcctcagggccgcctgcagggactcagggtggcccaaggctgcggtcgggcaccccgggccccggcaggagacagagacagagtaggaggagaagacggaggaggccatgacacagggcgtgagaaacacgagaacgcaagaacaggggagaggtggggaaaccaggaagagacagaaaggagagagggcgaggaagggggtggcgattgtctccagaaaatcaacctcgcattctgtcttagtcatgtcttaggtgtcaggctgaagtagcaaaggaaaagtgtgtagtttggagaggatgtgcataagaaataaaatagtccctgcgtaggctgaggagcatatacgcatctgtgtttgccacagaatcgtgaaatgtacatgcaccttactgtt

SEQ ID NO: 71 - DNA, 484 bp, Homo sapiens
acagaacaattcacctctttatcttgtgacacctacgagcgcatcaattctgtaattgaaaaataaagtgcatatttgcagcagctgtactctcttcaggctgcaaggaggcttttcctcccggtaggcttgatttgcatttcactttcactttcgtggctggaaactttctacccacgtagtgggaggctgaggagccaccataaagctggggcttgacgagccgggaccgggacccgatctccacatatgcccggacttgttctgcggccgggttcaggagtcaaagaggcggggagacctgcgcgacgctgccccgccctgcgcccgcttcctccaatgtatgctctagggggcgggcctcgcggggagcatggacacgattggccctaaagtcttccccgcaaggccgtgggctggacagcgtggtgacgtcgcaacgcggcgcagggtgagagcgcgcgcttgcggacgcggcggcatt

SEQ ID NO: 72 - DNA, 185 bp, Homo sapiens
cttaaccgcggatggccggagctggcgccctggttctggaggtaaccggttactgagggcgagaagcgccacccggaggctctagcctgacaaatgcttgctgacctgggccagagctcttcccttacgcaagtaagtagacgttatttaggtcgaatggaaaggccaggtgagccgacctggtt

SEQ ID NO: 73 - DNA, 801 bp, Homo sapiens
aaagttgcttttctctttggaaaaaataaaatcaaaatgctttctctgcgcttcttgaagcaatgaccctcaaaagcccagaggtattggccccctcgggggacccgggggccgccaagcagggttcccccaggtgggggctgggcagctggcgctccccgccgggccccaaattccagcgccgggccccaaattccagcgcctcccccgcgggttcctggacggctctttacgctcgctaaccgggcttgcaattttgcgctcgtccctgagccgggaaatcaacgaagttcctagtcgagatctgcccggtccgcctagtaacagcgccgcgcccccattggctcatgctaattccagtttcctctgtcttgcgcccgggatgggggggtgaagctccctcctggacccagagccggttgtgccggagtgggcgagcctctttatgccctgctgcccctagccgacttcggcccgcttcgcgcctcgggctgggccagggcgcacgcggggctcggggcccctcgccccacgggatgggagaggccgggtgatagctccgggccccataaatcatccaggcggccgccgggtcgggattttatgaatgaaaaagcagctgggccgcccttgtgcgcgggctgatgctctgaggcttggctatgcgggggccaacgcgattgtgggtgctcggggagtgggggggggcacgaccgtaggtgctccctgctggggcaacccatcgctccccatgcggaatccgggggtaattacccccccaggacccggaatattagtaat

SEQ ID NO: 74 - DNA, 853 bp, Homo sapiens
agagacgcgacccgcaccgacgcccccgcgaatgcaccgcgaatgcacgtgaccgcgcctagtggcgccccctcagtcaacgccgtcgcagtgcccctcccgaagcccgccgcttccgccggcccgcgcggccccgcccccagccgcgaggaaccaatccgcatgcacacttcttgtccccgccccggcaccctcctcggcgcgcgcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtggtgagtgtccgcgaccggccacgcccggcccccccaccgcccggggccgcagcggacgcagcctcggcctcgcccgcgcctcggcctcaggcgccgcggcgacctcgaacccggacttcaccgcggtccgcgcgccagggctcgggcgcggccccgggccggttgaggtttaggtgttgttgctcgcggttcccgggccttcggtcgctcgggaaggggctcagggagtggcgcggggggcgtaggccggtactagtggacccttggccccgggcgcagcgcggccttt

SEQ ID NO: 75 - DNA, 482 bp, Homo sapiens
aaaagaaaaacaacagtaactgcaaacttgctaccatcccgtacgtcccccactcctggcaccatgaaggcggccgtcgatctcaagccgactctcaccatcatcaagacggaaaaagtcgatctggagcttttcccctccccgggtgagtggcgacggccgaggcccccacccaagctctcggccagaggccgggggtcccctggacttcttccccctttcgccgttcccttctgggcggtgcatttcttcaggagcgggggatgctgttggcagctggatctggaggacgaggaggagaaggaggaggaggaggaggaggaggactgttggtggccctagacccttggtttggggggtggtatttattttcacagctgtgtttttgtgagtggtggggaaaagcttatagaatttccagcgccccattcctgctttttttattttcccaaatgagcgagaaccctctggccttattcctt

SEQ ID NO: 76 - DNA, 141 bp, Homo sapiens
aaagacagggtctgactcaagtgtctaattacattggctaatactcccagtacaagcccaagaacaaattataaattactctaagatgaatggtctctttcattgcttagtcatcttctcagtgatcaagtagttatctct

SEQ ID NO: 77 - DNA, 468 bp, Homo sapiens
agctgggggaactggggctgccaccagggggcgcgaggggccttcgcccgagaagaggggtgggcaggtgcctccagcggagaagggcgccgtggccggaggcacaggtctccccggtgccacttcaagtgagttcgaggaagtacctgggatctttgatctaacgcgaaaggccttcccagtgacctcttgagagctgagaacccactccctccacctctagtccacggctttgccactccagggcccgaggttacgtttgctgctggggatttgacaaacccaaagcctctctggtttcaccactggctccttagaatcagacatctgttctgaatgacacttatgtgagtcaggggctgaggacgtgatcctcgaagtgtggtccccagactggctgtatcagtgtcggcatcccccaggacctggttggaaatgcatattctcaggccctactccagacctctt

SEQ ID NO: 78 - DNA, 212 bp, Homo sapiens
gcctttggggaattcagagcaaaccagcggcatttgttgggggtctccggccttcaacatcacagacaggcctgggggtggccttcccaaagtcagattgcagatctgaggcagtttccccctccctgcgtccctcactgaaaccttgaaccccattgagaagtccctttagggtttcggacgcctccacctcaccctgggctggtgcttaa

SEQ ID NO: 79 - DNA, 61 bp, Homo sapiens
atatcaaagacatcgccagtaactcagaaattagattttattttgttttatttactttttt

SEQ ID NO: 80 - DNA, 66 bp, Homo sapiens
aatgtcacgttgaggaatctcactctcagcatgttagcaactcttctatatttgaagacatatatt

SEQ ID NO: 81 - DNA, 109 bp, Homo sapiens
aattacaaaattatcaaaagtattgatagtaatttggttagcatacacatttctataaagaaatggcatattatagaatgtttgtctgtttaccctttcctaaaattaa

SEQ ID NO: 82 - DNA, 454 bp, Homo sapiens
actgtgcggctccgagttgagctatgccgaaaagcccttttcagaatttctgcaaggaatgtaatccaaattagttctgcagctgtcctcttcctcgtctcaggcgacactagctcgcgccctcgcttcgtctcttctcgtttccctgggcgaattgtcagacccacaagtgagcccatttagagcgcgaggattagcgtgccattgttgttccgtgtgtgactctgtacttgaaattctgggggcacaaaggcgagcctcacttgttcaaaacacaaagtgatccagatgaaagggccgcacaaagaaaagctctcgggtgcatccctggtataattgcaacacgcatactaaggtgatccattggaaactgtaaatgccccgggggacttcgtttttcatcctcccccacccccaccctgccgcacctggcagggaaagttcagctggtttt

SEQ ID NO: 83 - DNA, 145 bp, Homo sapiens
atatctgtttccctcaaagaaactatcagatgcatggcaacaggactttgtctggtagttaccctagcacataggccagtgcctggcaaaaaataggcaactggtaaatgttgaacaaatgaatgaatgaatgttattatggaat

SEQ ID NO: 84 - DNA, 33 bp, Homo sapiens
mccctagcagcaggatgatcaacatratacaat

SEQ ID NO: 85 - DNA, 832 bp, Homo sapiens
awcagccccggtgcgcagctaggtcggaaggcgagccgcggagagcggagccgcgggccaaggcagggacgggaggggaggcggcaggttgggcgctccgctgctgctcggtgctgcttccccggagccgcttcctcatcctgcgggggctgccccgccgccggggtgccggggtgagcgcggcgggagccagggctggggtggcgaacctcggagggggctctcccaggaagccctgcggttagcgcccgcgcttccccaccagtggctgcgcaagctgggaagcgaagaggtcacgtgttggcggaaagtaacttggaggcgaaaggatgaactttgagaagaggaaaaaaagcacaatgaaacaaactacccccttggagtgtctaatagagggggaggcgggagggggggcggaggggcgccgctctccgcgggagatgtggggacgactccggaaatctcgggaaataggcaataactcgagctttggcaacgtcccccagcctagggaggcgctaacgcaaggtatctttccactgaagctgccctttgcctaccctcagccatcgctagcacagcagagcagaagaaccagggatgaatgtagaccagttgttatggagatgtccgtcgctctgaggagcgctagtgccctgggaagtccccagacgcatttcaggtttcgtcgttttggcactgacaccatgattttactcgctctgagttcccaccaaggggagcccaccaacggcgtcacaggccctggccgtggcgctattgactgcagggatgaggcacagcgatgctgcagaagatggtggatagctct

SEQ ID NO: 86 - DNA, 416 bp, Homo sapiens
tatcacagagcaagggagttacaggactcgctcgcagtttcggaggctgcagagtagcgtataccattccggggcgcgcgtggaagcccgacccaggcagcgagggcaccggtacccgccgggtctctcccgcccaagctcaccctctccccatgcctttgttccgggcggagcggctttcccgagctctcacctccggtcccgggggccatccctgcactccccgaccggagctcggcggagcctccacttccgagaccttcgactggcctcgacccccactgggccccgaatgcctcgacccccgagccctactcggatgagctgtaggggcaccgcttgggcaaggtgatatttccttttgctccgggctccaaaatggcttacgatggcactgcacggatcctttctctttt

SEQ ID NO: 87 - DNA, 99 bp, Homo sapiens
atacaactattactctacggaaatattaggggtagcgcaaacaaagttctgacactaaactttactgagccctgcatcctgcccacgacccccccagaa

SEQ ID NO: 88 - DNA, 746 bp, Homo sapiens
gagggcggcggcccagccctggtccacccacgcgcggccgggacttgcgcccctacccccgtggccacgccctccagccccccgggctcacggcctcaactctcgacccagccccctacttctctaggcctgcaaccgcgcagccctcacccctcgtacccggggcctaagcccaccccagattctgggtcattcggagccccagtgcagcagaagtccggctgcgggttcaccttgtcctccggcacagggcccggccaccaggcgccacgctggatagcctgcggccctcgcagggtccttcaggcgctccctgggcagccctgctcggggtcctagtccggttgcgccgcggcgtgaggtgaggtgtgagttgccctggagggtggcgggttttccaggcgcatgcatttgccgcggacgggacgagaggatgaagccgttctgggcgccggaactcgcttgtgggaaccgcccctcggaagccggcctgggcgccctcggaagccggcctgggcgccctcggcgggagggagtgctgggccgggtgctggcgcagcgctacgcagggtggcggaacccagaggaccggacgggctcgttcagggtggcggcccggcgggggcgatgcccgccagcagagacaccgatggaaaccttgcccagttattttctttgcccagggtagttcctggaacatggttgatgcccagtatttgtcaaaagtacgcggagggcaaagggct

SEQ ID NO: 89 - DNA, 25 bp, Homo sapiens
gggcccccccccccccccccccccc

SEQ ID NO: 90 - DNA, 1936 bp, Homo sapiens
agaaacgatctcaggccgggcacggtggctcacgcctgcaatcccagcactttgggaggctgacgcgggcggatcacctgaggtcaggagttcgaaaagagcctgaccaacatggtaaaaccccgtctctactaaaaatacaaaatcagccgggtgtggtggcgtatgcctgtaatcccagctacttgggaggctgagaagggagaatcacttgaacctgggaggcagaggttgcagtgagccgagatcgtctcaagacaaacacctcaaggaaaaagaagaaacgatctcttcccttctcaaagttatatacatttcactattcgccaaccacctcctccacgccgtccagagctgagctgacacgcagcgaagtcctctcagtggtccctccgcacctcagccctgtgcttggcagcgagacgagtgacagggccgccccagcatgggtctcccgggccgcggacccgccccacgccgtatccagcacgctgtcctcccgccccatctttgtgccggccatcgtgtggtcgcgtctgcgcgctccgcccggtctgccggcgtgagaaagagctcgtgtcgttcccaagttcagaccagaggggacggcgaagcgcgaaaaagttccctgcgcccagtacgcaggcgcagccgtgctcctgggcttcccgggcctcgtcccagctggcgcgcccggcgctcctccgccagcgggcagttgggggcgctgcagggagggcacccgagcgggttgggggcgctcggaagggtggcttgcaaggtccgggtagctgcagtccccggagtggggcggagaggcgcggcaccgcgcggacgaggatggggcgggggcgggcgcagcccgagggtgcaggcacgataaggcgggtgacagccagcggggaggggcgggcgccaccgcgtccacgccctttgctcggggccgggccgggagggtcctcgggctttgcgcgcgcctggccggcgggctgaggcgtacgggtcgcacgcagcgccatgcgaggcccccctgcctggccgctgcggctgctcgagccaccggccctgccgagccaggtcggctcctgccggtagcctgcgtgtgggcgcggccagccgcgttcccgggtccctatcgccgttcactggcctgaggccggcgcggctatggggcgcggggcccgcgctgctctggggcgttggagccgcgcgccgctggaggagcggctgccggggcgggggtccgggcgcctcgcggggcgtcctgggcctcgcgcggctcctggggctgtgggctcgcggccccggcagctgcaggtgcggggcttttgccgggccaggcgctcctcggctcccgcgcgcccggttcccgggcggtcccgcagccgctgcctgggcaggggacgaggcctggcggcgcgggccggcggcgcctcccggggacaaggggcggctgcgccccgcagcggccggactcccggaggcccggaagctcctggggctggcgtaccctgagcgccggaggctggcaggtagggtcctccccggccacttccctccgccgggcctctccttctccacacgcggggtcccggggcgccgagagggacacggccgggccgcagagcgcgtgtcaccgccgcctctcctgggccactcctaaccccggaggagggaggggatcgctcacctcagagtagcgttgtcaagatttcgccttagctggggactcgtttgcagattttacacttgttttcttgttgacacacgttatatatgtcattattggaatctttgggtctgttccaccaccaacttagtgggagcatctgttttggcttaggcagcaaaatgagctggaggtttttccttggaaaaaaaaattctagccaaaaatatgcctattttgttattaggcgtttatactttttttttt

SEQ ID NO: 91 - DNA, 223 bp, Homo sapiens
aatgctgcagccactgcgcggaagccacagcgcctgccagtgccgggagccggggaccggggagtgagtaagcgggggagatccgggaagccgagacctgggggtctgcgagccgcctcctcccgcgccgagagcaccgggctaggagkccggacaggtggggaaggaactggaggaascgggcgggagrcwtcctggggasggcgggtrggcgagtstgagt

SEQ ID NO: 92 - DNA, 49 bp, Homo sapiens
aatatatggtctgcaagcttttcaaaagaatatgacaagaagaaagctt

SEQ ID NO: 93 - DNA, 652 bp, Homo sapiens
aaaagtcgggaaacaacagatgctggagaggatgtggagaaataggaatgctttccactgttggtgggagtgcaaattagttcaaccattgtggaagacagtgtggcgattcctcaaggatctagagccagaaataccatttgacccagtaatcccattactgggtatatacccaaaggattataaatcattctcctataaagccacgtgcccacgtatgtttattgtggcactattcacaatagcaaagacttggaaccaaccaaaatgcccatcaatgatagactggataaagaacatgtggcacatatacaccatggaatactatgcagccataaaaaggatgagttcatgtcctttgcagggacatggatgcagccagaaaccatcattctcaggaaactaacacaggaacagaaaaccaaacactgcatgttctcattcataagtgggagttgagcaatgagaacacatggacacagggaggggaacatcacacaccagggcctgttggggagtagggggctagggaagggatagcattaggagaaacctctaatgtaggtggcgggttgatgggtgcagcaaaccaccatggcacgtgtatacctatgtaacaaacctgcacattctgcccatgtatcctagaact

SEQ ID NO: 94 - DNA, 174 bp, Homo sapiens
aatgcagttcactgctagggtgccgactcactgagcacgcaggggcggaggaattcattctgagttggttgcaccgaagaaataaagctcgagcagcttgttttccccggcgccgtccaaggggagggaatagggaattggggtggctggggtggggggtgagggcggatccct

SEQ ID NO: 95 - DNA, 539 bp, Homo sapiens
ataaatgactgggacagcgcgctctcccctagcgccgcggggaagctgcgggagccggccgccgccagcctgcggagaggccggagcgcgccccctgcctgccgcatcgggaacccccgctttaccgggcgctttcgcatgtggttccctccaggtgcacgcggtccccatccctctgcctcgacccactcgagtctagtgacgagcactgtgctggggactcgaaggtggagagaaaggagaccctggcaatatgtgtgtgttcatgtgtggggcacatcaggtgctgagggaggagccccagagcctcattagcatggccattagctattcattaggctgcagtcgaagcatctcctgagaccgggaagggtagagggggaaattccagggaaagggcgcctgcagctgtgtccacaggaattagggggaaaaggccgaccagaagggtgtggtccgtatagaaggattggggagaaagacgatggggtaggagcccgattctccaacctcttgaccctcctagatttccaggaggt

SEQ ID NO: 96 - DNA, 136 bp, Homo sapiens
tctattgaaaaaatacataaccacaagtgcctttgccccatgatgctgcttcctgcaactgacatcaggaatgcacggaaaaagaatggagagaaagtgattcctattctctgcaccctttccgcttgccaggctt

SEQ ID NO: 97 - DNA, 1245 bp, Homo sapiens
  misc_feature (67)..(67): n is a, c, g, or t
taagttctggcccatggacggtcaatccgatcagcgcgatgtcagttctagagaggcagaaacagancaaaggaaagagtcagnaccgctcgangcgctgcaacccaggggtccggtccttccacatttggccgtgactgggagtctgcactttcctccgcgcctcccgcgggcccccagaagccgggcgcgcacgctgcagagcctcggccgggcaggcggcccccagccccgccctcgcgcccggctcccgctggcacgcgtcccgcaggtgtccctgcagactcggcgaggcctggccgcgcctcctccctggccctcgaagggacccgccccgccccatgcctgcgcggccgagagggcagggggccgggcccgcagggaaggagctctgggcccgagacgccgtagggccagggtcacagagccccaagtgagctcaggcgacccccagggtgggaggcgtcggagagaacgagggagatctcgacccccaaagagttccccgcccgctgaggccgagccgggctgcccctggcgagtcactcactgggccatggcggcggacagagcagaaccgaagagcggcgacgcgcggaggacgagtgagggaaagacccgcagcggctccctccagaaactgcggccgctcacgcccggcttttcggcgccggccaatcgaatggaagggggtggagccatgaagccacaccattggctctccccgacgcctgttagaccatccgaggcaaacgtgcccgcccacccccgggggccggatcagtggagcgccgagcacctagatgacattgccccggcgaggctccgggctccccgcccacgcgcagagctttccgctcgtcccagctgcacctcggtcctgcaggccgtccgcctggcgcagctgtagccatagcaactcacgcaggaccgcgctttacagtttaccaggcactcggacgcctttggcgcgagcgcccgtcgctccttcctcaggtcactgcggtgcaccgtgcgggtccgcgtcgtgtcctccgctccggagcagggaccgggcgtgctcgttcagtcctgtgcgcactcggaatggggcggggaaggctgagcctcaggncgaccctgatgtggagccgtgcgaccgctcactgcggttgaagagacccaggcgcagacagcgcaggcggggtatgtattggttggagtatgatggggtcaggacttgagtcttttacccaggcccggagt

SEQ ID NO: 98 - DNA, 725 bp, Homo sapiens
cttatacttcccgaggtcgcctctctgccatcaaacgaaaacatgcaagaaaataaaactacacatacgaacatgcaaaggaaacaaaaccaaagaaaaacaccaacgcactcgttgggggaaatccatggcctcctgttgccagtcgcccggcgcggaaagtggacagcccagcggccagtgagaagcaagagcaagaaagcaaaccttctgaagacccctctcctcgcccagctccgccgaccattctaccaagctgccctcccagaaaaaagagcaggaaaaatgcctgtgcaaacgtgccttggcgagcggggaataagtgggctgggaagaagggaaggattcggaaagcacagctgtcccggggaacaggaaggaggagaaagtctctccctcccttcagtgccccgcgccccagcgaggaggaagattactagtccagcaggctgcgtccagcttcctacacaggaacctttagcatgggcaacggaagggcagccccctcctgccttcccctccccaccccaccccctcgtcccatgtccccgccacagggagagcgycgsccgggagaggagaagrccgagcgtgattgtcccgcarccctmgcytcagtcggckccggggccsccktgkaccckcgagtggtgcagtgggcgaggccattggcaagccagggatcaggagaatcccagcacaaccgagggtcgggcccgactaac

SEQ ID NO: 99 - DNA, 255 bp, Homo sapiens
aagatggggaagtagcagacacccacgcgtgaaggcaggagagccccaactgtggtggaaatggccccagaatggtagggccaagcctagctccagacaccccagagccctggagaagccaagactgagggagaaagcctgagggaggagcgccccagtccccagggaccggcctggtgcagagctgcagctgatgttcccctctgtgcagccccaccctctgcctcgctgagctccctgctgcgagggcctcgg

SEQ ID NO: 100 - DNA, 508 bp, Homo sapiens
agcctaatgtggggacagctcatgagtgcaagacgtcttgtgatgtaattattatacgaatgggggcttcaatcgggagtactactcgattgtcaacgtcaaggagtcgcaggtcgcctggttctaggaataatgggggaagtatgtaggagttgaagattagtccgccgtagtcggtgtactcgtaggttcagtaccattggtagccaattgatttgatggtaagggagggatcgttgacctcgtctattatgtaagggatgcgtagggatgggagggcgatgaggactaggatgatggggggcaggatagttcagacggtttctatttcctgagcgtctgagatgttagtattagttagttttgttgtgagtgttaggaaaagggcatacaggactaggaagcagataaggaaaatgattatgagtgcgtgatcatgaaaggtgataagctcttctatgataggggaagtagcgtcttgtagacctacttgcgctgcatgtgccat

SEQ ID NO: 101 - DNA, 25 bp, Homo sapiens
gggcccccccccccccccccccccc

SEQ ID NO: 102 - DNA, 255 bp, Homo sapiens
  misc_feature (203)..(203): n is a, c, g, or t
aaaaaagtcattttgatggatactcccaaagagaagagactcagagcctggatcccaaggggtaagagaaaatatccttacagtaaggggtttcaggagtgctggcatagaaaccagtttctctctcacacatgctctttctctctctctgagatgcaagatcaattactgacgttgctcaagagagaaggtaaatgagaaanaaggtgaaaggcaaaangaaaagaaaggtaggaaaggaaaaggaaagaaaga

SEQ ID NO: 103 - DNA, 254 bp, Homo sapiens
  misc_feature (50)..(50): n is a, c, g, or t
taaatcctgatggaactcatccgtaaagcagatgcccaaaggatgatagnaagcttactgtgacataactcccggagccgcagcacgagccttgtgaaccttggaagcccgttttccgcgcagtcactaactcgcttagtcaccctgtgtaaaagttattctgggatgacacctgcctgggaagtctcacctttccccccacctcctcctgtctgngtattgttcttgtagcaccccggtgcgcaccttgattg

SEQ ID NO: 104 - DNA, 778 bp, Homo sapiens
ctttaggagtcacacccatcctccctgcttccctggtcacagtctgccaagaaacgccctatttcaggagtgattaccctgcaccaggcatgggtctaagtgtgtcatgtacattatttgacaaatatttacggagttaccatcaggtgtcaggcactgttctagaccctgtggatgccactatcagctaactggaaatgcctcatctccctgcggatgcatcattgtcctggctcgtgttcacagggaccccccggctctgtaactgtgccatgagccggctgaggaaatggcgctcaggcaggcaaagtcacttgtcaagggcatgcagtaagccaagamtgaggaagstctgtstccaagcctatgcscccracccagggatgtcacaskmccagmcacgtgccaggtccaacccrccacskgcttttgtawatagagttttattkwcmaccakgsccacacgtttaggtatggtctctggctgttttcatgctacmacgasggagtagtgacaacagasactgtgtggcmtgcaaagcctcaatgattcactcgctwacsscctgcagaaagwttgttgactcccccgmccactgtcmtcrccamtgcagcgstgccacttttcgaggaaataaaaatcatcccatcatatcsggsctcaaaaataccccttgwttctatttttmtaaatttccttcctctttttctttcctctctgtacrtgwtccaatacaaagaaagccttttatcttataaatgagggtaaaaatagtac

SEQ ID NO: 105 - DNA, 1036 bp, Homo sapiens
ccctttaggctttcatccaaaaggagaaaacataagcacagcccattttcagaatgaatagtatttttttctcttttttttttttttgtagagggaggtggacgggggaggagacagcggtgcaataaatggtcggagaactgactgaccaatgttcaaccttgactgcccctcattcggcttccagaggcacagagagactgaggggccttaggctggaccgtggaatcaacatctagctgcgtgagacccgaacacggagacgcaggcccccgccttcatcgctcgccaatggggggcgggcaggagaagacggtgggcctctccctgcccctggtgttgctgacgggggccaggaagccgctggcagcaggcagcggccaccggagagagaggggtgcgggtcaaggccagcggtggggtggcccgccctgcgggtgtcgcgcaggagggtggggatcgggagccccagcttccgcggcctaggctcaaagggcggcccagcaggcaggccccgaggggagcacaacccgagccccgagggcctgggcgcctgccgagcgactccaggcgagaaaagcaaggaattcggcgcctaggcagcgcgcgtttccaactgccagacccagctcggaggcccgaaactgacctgcttttggggcgtccatcgccggagaccatattatccctccggggagggggccagcgggggaagaagggggaggccgggcagggaagacggggaggaagacaggcggactggcmagggaggaaagaaggaagcaggctcgcgcaaatatcgcgagagcagcagagccagccccgggtctgcgctcgcgggatttgtcgcctaactcccgcgagatcgtcggacaggaaaacaaagaaagggaaggggcggagctaaccagaaagcgatttctcgcgccgtcgccgctgttctcgcgagcgggggcggagcgctggggaggctccagtgggggcgttggcgtggctagtttggccttcgcagcgatccgttacttagttacggcct

SEQ ID NO: 106 - DNA, 428 bp, Homo sapiens
cttgcagagaatactcttgagagcgccctccctgatctcggccagctgatgacctgccctcgaacggcacataactattttatttcccgactgcctagagactacgttatttatgctgtgggcaggagcgtcgtgcagacggaatatcccagtttttttcgaaagcaggccactggtgccttcgggacgcgaaccgccaagtttacacaaagcccaatatgggcccccggttgctatcctgactttccggtggtgaaagagggagcggccgcggaccggccggtcgcccaaacgtgacacgaccattctcatccagtgtccagaccgcggggggtgccttgggcgaggggcaagctgacagttaccgagtttcctgatgagccctgccaggaccatctcgtcccgtcaatctgcagccgccggagaat

SEQ ID NO: 107 - DNA, 981 bp, Homo sapiens
aaggaaccagtggcgctgtccgaatcagcttttccccaacgtgactgttgctgtttcccttattcactcagattccgcagggcagcctccagcaaggctgcagaggccaagaggagccaagaaattctgagaccaatgaaaagtcgcgtccggagagcaggggcggccctaacggaggcagcctgcccggatacccagggccgagcaccgcggcgtgcctggtgggatgaggcgagggcctgccgcaccgccagggaaaggccgaaggacccggaaggaagcgccaggcgaggccggggcggaggctgggaagctgcgccctacccggggaaagaaggggcccggcagggccacgcgaggcttggaccgcggggtcgctctcgcacagcggctgcaccgggcgggcgcgccggcttcgacggtgctcggggggctgcggcctgtcactcggcccagggcccgccctccggccccgccgccggcgctctcgggcacacgggacacgccctggccgcaggtctctgcccggcgccgcagagccccggggaccgaaccggcgcgacccccgcccggcagcgtaaccacagccccacctaccgctgacgcggccgctcgcctcgccccgccccttccggcggggggaccgcccctcttgtcaggtgcccaagacctctatcgccattggctggccctgggccggacgcatcaggacctggggtcaccacggcgacgactgcacctaccgctcgacgcggcctttctcgccgttgccccaccctttccggtcaaggccccgccccctatcacgtgccctgagctcgacagccaatggctcaccccagcccaggaagcgcaggcgcaaagacgcggccgaggtctgcgcggtgacgtaggcgcgctcccggcgagggcgaggcaggctgccgggggtggggagggggacgcacgagaaaaaagaggcgggagggcgcagaaagtcct

SEQ ID NO: 108 - DNA, 101 bp, Homo sapiens
aatatttcctaggaagaaaatctgtccaagtctgccaataaaccatagggaaatacacaggtatgtagaaacgtctggattttcataattacacgggagat

SEQ ID NO: 109 - DNA, 172 bp, Homo sapiens
atcaattcagtcatccctatctttctccttacatatcaatcctgtagattagtgactcttgtataagacaagaaaaaataatgtgcctgtgagatatcaacacaggtcagtctctaagcagaagtgaaaatatggagaaatgagttggaaaggaaaatgttatagaaaatat

SEQ ID NO: 110 - DNA, 937 bp, Homo sapiens
ataaaacaggttgcaaagtgattttagaaactacaggaccctgacccgggtgctggggaaagttccagctcctcagaagttgcagacactgagggtagcccgctggccagaactcccagcgcgctccctcctcgtggcgtccagatgcccctcagagaagtcagggacagaagaaccctctgggctgcctggcggcaggataaaaaggagaagccctgtctcagggacgccctggcctcagtgcccgagtgcagttgtctaacgatggcccgtggtctccgcagagccccgtagcccggcctcggcgtcaagcccctccgaatgcgcgactacccacctgggggcgctgggccgcagggggcggggtccgggctcccaagccccttgcctggcgctgctggcagcattgtggcggttgaagccgaggggcctgcgcactgctgcccgccgagcgctacctggactctcccggccagagactccccgcgattttctcttcctttggccctcggctctccccagcccgcttgccacccgcggaggctgcggccaggtctcaggaaacgcagccccggggctcatgcccgcaaggccagggctccgctctgctgggcgcccagaggaagtgaaactgctcaggggcctgctcttacatcccttctcaaaaggccgctttggtttcttataacacttctgcgcctgtttccttcctcaccttttcatcctaggatatatttagaccgctgttattgtagctctccttgtccctttctaggtcttccatactgccatactgcccccacttccaggcagacgtagggaaaccagaagtgaacatttgctggaaagaaagaataattagatagatagatgagtaggtacacagatgatagatacatacatatagaaatgaagatacagataatactgtaacttt

SEQ ID NO: 111 - DNA, 693 bp, Homo sapiens
aggcggcggcgcctcgcgaaacgctggtgcccggctggagctaggcgccagcgctgggcccggccctcactagatgggcgggtgtgtgtgtggccgccgccctccagggaagcggccgggagtgactcaccctggctggctccccagcgcgcagcctgcactggaggaggccgggctgcctccgcgcggagactcccctcccgcacacttctccgctttcggcttttccttccacctcccgggtacagcagacccgtccttccccagtccaggctgctaggccgaggggaagtggcgccgggggccggggacaacgcgctcacacacgctgtttcgtggcaatgcgccctcgagagcctgactcccatcagccaccttggctggaaaggggtgagggtgggatgaagcgcccagccgcctgcgggttggcaacaaggcctagcgaatgtttctcctagaggagaaacaactttaccggcgtggcctggtgtagcccgggagagggtggagacaacggctgtgacgggcttcccggctgccaaagggcctcattcattcattcaaatatttactgagtgtctactaggtgccgggctgtaggtccccagagataccaggtgaacacgaccccacacactccttgccctactggattgtattctgatggaagcgacaaaacaacaaacaacgctt

(19) United States
(12) Patent Application Publication
(10) Pub. No.: US 2013/0102483 A1
(43) Pub. Date: Apr. 25, 2013

(54) METHODS FOR THE ANALYSIS OF BREAST CANCER DISORDERS

(71) Applicants: Nevenka Dimitrova, Pelham Manor, NY (US); Surabhi Khandige, Manipal (IN); Satyamoorthy Kapaettu, Udupi (IN); Aparna Gorthi, San Antonio, TX (US); Shama Prasada Kabekkodu, Kumbla (IN); Sanjiban Chakrabarty, Manipal (IN); Payal Keswarpu, Bangalore (IN); Nilanjana Banerjee, Armonk, NY (US); Angel Janevski, New York, NY (US); Prashantha Hebbar, Udupi (IN)

(72) Inventors: Nevenka Dimitrova, Pelham Manor, NY (US); Surabhi Khandige, Manipal (IN); Satyamoorthy Kapaettu, Udupi (IN); Aparna Gorthi, San Antonio, TX (US); Shama Prasada Kabekkodu, Kumbla (IN); Sanjiban Chakrabarty, Manipal (IN); Payal Keswarpu, Bangalore (IN); Nilanjana Banerjee, Armonk, NY (US); Angel Janevski, New York, NY (US); Prashantha Hebbar, Udupi (IN)

(73) Assignee: KONINKLIJKE PHILIPS ELECTRONICS N.V., EINDHOVEN (NL)

(21) Appl. No.: 13/641,282

(22) PCT Filed: Apr. 8, 2011
(86) PCT No.: PCT/IB2011/051517

§ 371 Date: Oct. 15, 2012

Related U.S. Application Data
(60) Provisional application No. 61/324,797, filed on Apr. 16, 2010.

(51) Int. Cl. C12Q 1/68

(52) U.S. Cl. CPC C12Q 1/6886
USPC 506/9; 435/6.11; 506/16

(57) ABSTRACT

The present invention relates to methods, arrays and computer programs for assisting in classifying breast cancer diseases. In particular the invention relates to classifying breast cancer disorders by determining the methylation status of one or more sequences according to SEQ ID NO: 1-111. The classification may be further strengthened by also taking the expression levels of one or more proteins into account.

### FIELD OF THE INVENTION

The present invention relates to methods for analysis of breast cancers using methylation patterns.

### BACKGROUND OF THE INVENTION

Currently there are epigenetic studies available that show the relationship between gene promoter methylation and cancer. The promoter regions of most housekeeping genes and about 40% of tissue specific genes are characterized by such CpG-islands. Methylation in these CpG islands is generally associated with gene silencing. Programmed DNA methylation plays an important role in normal embryonic development where waves of global demethylation followed by de novo methylation characterize the early pre-implantation development. During tumorigenesis global DNA hypomethylation has also been reported, which results in chromosomal instability and expression of some repeat elements (such as transposons). Hormonal influence is reported as common to all women's related cancers including breast cancer. The research focus lately has shifted from genetic to epigenetic factors as potential biological mechanisms. This in turn makes these epigenetic mechanisms conducive to being explored as potential diagnostic biomarkers. Tumor suppressors, oncogenes, and other cell signalling genes have already been studied individually for promoter methylation. In these studies, there are different levels of sensitivity and specificity reported for various genes.

WO 2009/037633 discloses method for the analysis of ovarian cancer disorders comprising determining the genomic methylation status of one or more CpG dinucleotides.

The inventor of the present invention has appreciated that an improved method for classifying a breast cancer disorder is of benefit, and has in consequence devised the present invention.

### SUMMARY OF THE INVENTION

It would be advantageous to achieve an improved classification of breast cancer disorders based on determining the methylation status of one or more DNA sequences. It would also be desirable to enable improved classification of breast cancers by further determining methylation status of one or more DNA sequences and the expression levels of one or more proteins. In general, the invention preferably seeks to mitigate, alleviate or eliminate one or more of the above mentioned disadvantages singly or in any combination. In particular, it may be seen as an object of the present invention to provide a method that solves the above mentioned problems, or other problems, of the prior art.

To better address one or more of these concerns, in a first aspect of the invention a method is presented that relates to analysis of a breast cancer disorder in a subject, said method comprising determining the methylation status of one or more sequences selected from the group consisting of SEQ ID NO: 1-111.

In the present context the phrase “methylation status” is to be understood as the extent of presence (hypermethylated) or absence (hypomethylated) of methyl (CH3) group on carbon number 5 of pyrimidine ring of cytosine base in DNA.

The one or more sequences according to the invention may be positioned in or on a composition or array. Thus, in another aspect the invention relates to a composition or array comprising nucleic acids with sequences which are identical to at least 10 of the sequences according to SEQ ID NO: 1-111.

In the present context the phrase “composition or array” is to be understood as also encompassing University Healthcare Network (UHN) Toronto human CpG island 12 k microarray chip (HCGI12K). The methods according to the invention may be performed by a computer. Thus, in a further aspect the invention relates to a computer program product being adapted to enable a computer system comprising at least one computer having a data storage means associated therewith to operate a processor arranged for carrying out a method according to the invention.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows workflow of the Breast Cancer Study

FIG. 2 shows the steps involved in designing the CpG island arrays (From the original UHN Toronto paper).

FIG. 3 shows Volcano plot after t-test against zero mean null hypothesis for IDC vs normal.

FIG. 4 shows Volcano plot of T-test results IDC vs. benign with fold change above 1.5.

FIG. 5 shows Analysis on IDCvsNormal samples where p-value cut off <=0.05 relating to pre- and post menopause status.

FIG. 6 shows Fold change between Her2− against Her2+ samples in IDC vs. normal.

FIG. 7 shows Fold change of 44 loci between post and pre menopausal cases in IDC vs. normal.

FIG. 8 shows Fold change of between ER− against ER+ samples in IDC vs. normal.

FIG. 9 shows Fold change of between PR− against PR+ samples.

FIG. 10 shows Fold change of between ER−/PR−/Her2− against ER+/PR+/Her2+ samples in IDC vs. normal.

FIG. 11 shows clustering on IDCvsNormal samples after t-test post vs. premenopausal status, p-value cut off <=0.05.

FIG. 12 shows 24 entities which had a fold change of >1.3 depending on the onset of breast cancer.

FIG. 13 shows a clustering analysis of the breast cancer onset of the disease.

FIG. 14 shows an overview of key modifiers in significantly changed pathways in breast cancer using differential methylation data from IDC vs. normal samples.

FIG. 15 shows differentially methylated genes CCND1, BCL2L1, ERBB4 and PARK2 as being important hubs in the gene network of key regulators and targets.

FIG. 16 shows transcription regulators where ETS1 and AHR are being active in our IDC vs. normal sample set.

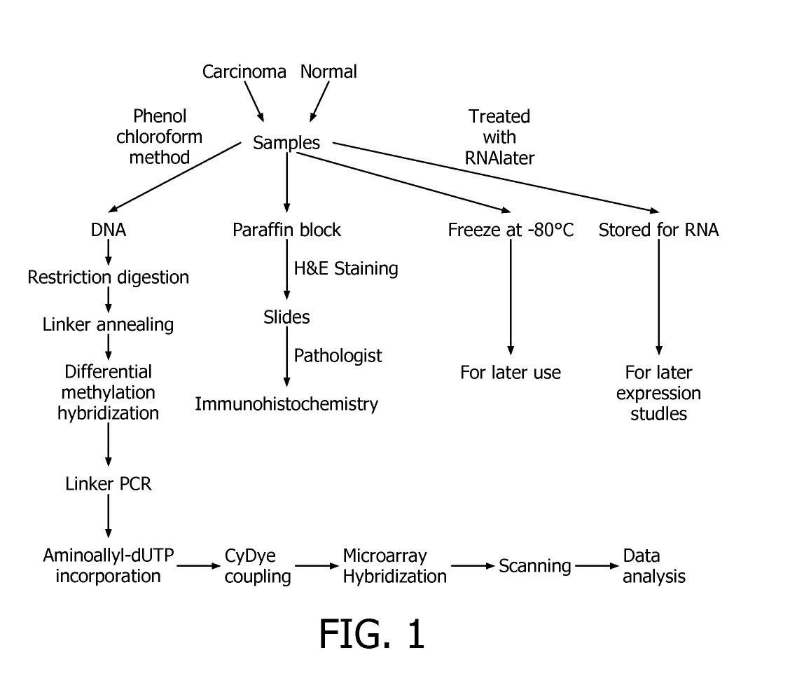

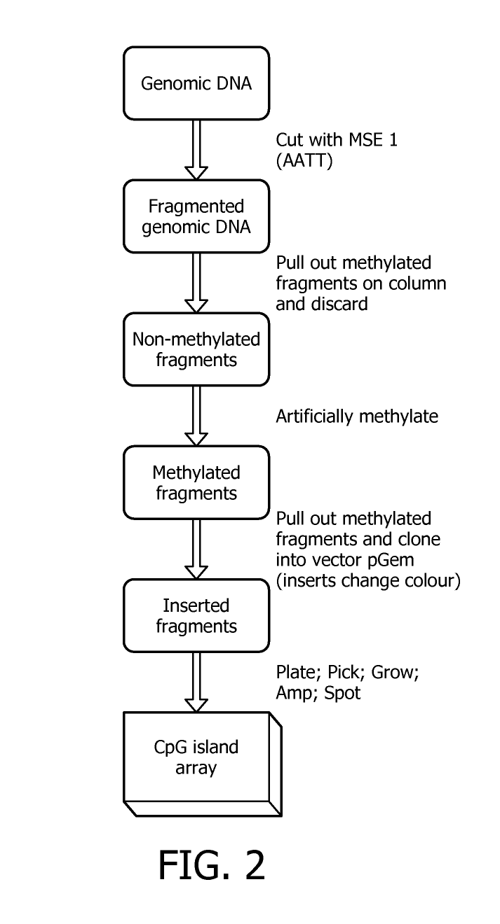

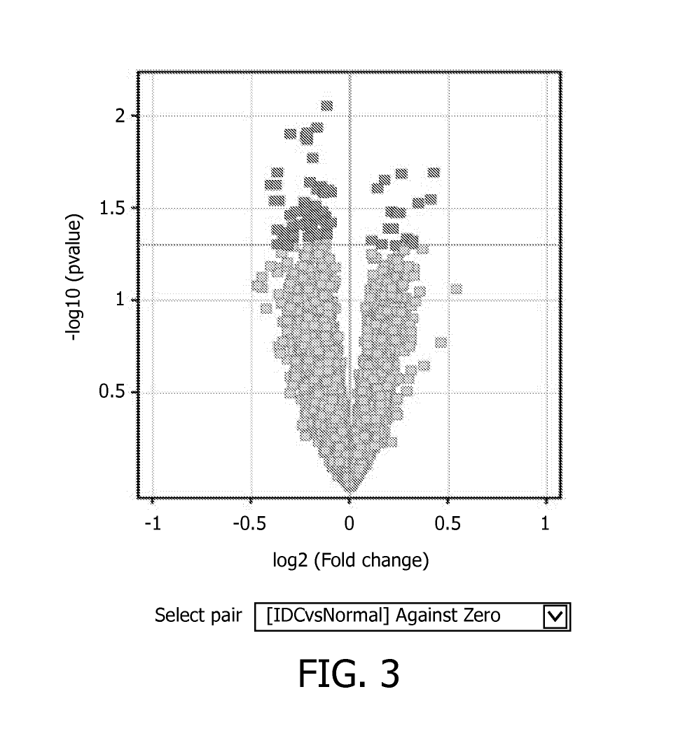

### DESCRIPTION OF EMBODIMENTS

### Method for Analysis of a Breast Cancer Disorder

The general aim of the study was to identify novel differentially methylated genes in breast cancer. Differential Methylation Hybridization was performed using a UHN CpG 12 k DNA microarray chip with DNA from breast cancer patient biopsy material as the sample source. The genomic DNA from the biopsy material from each individual patient was coupled with its corresponding normal counterpart. The DNA fragments generated as per the protocol were enriched for methylated fragments using methylation sensitive restriction digestion and subsequently the cancerous and normal DNA was labeled with Cy5 and Cy3 respectively. After hybridization the microarray chip was scanned and data analysed to reveal genes which showed differential methylation in breast cancer.

In general the present invention relates to determining the methylation status of one more DNA sequences in a breast tissue sample obtained from a subject. Thus, in an aspect the invention relates to a method for analysis of a breast cancer disorder in a subject, said method comprising determining the methylation status of one or more sequences selected from the group consisting of SEQ ID NO: 1-111.

The number of sequences to be determined may vary depending on the sample. Thus in an embodiment the methylation status is determined for at least 5 sequences, such as at least 10 sequences, such as at least 20 sequences, such as at least 40 sequences, such as at least 80 sequences, or such as at least 100 sequences.

In a further embodiment the invention relates to a method, wherein the analysis comprises assisting in classifying a breast cancer disorder, wherein the following steps are performed,
providing a sample from a subject to be analyzed,
determining the methylation status for one or more sequences according to SEQ ID NO:1-111.

The sample may be obtained from a human such as a female. In an embodiment the methylation status is determined for at least 10 sequences from SEQ ID NO: 1-75.

### Classification

The classification may be divided based on a multi variate model. Thus, in another embodiment the invention relates to a method, further comprising
a) the one or more results from the methylation status test is input into a classifier that is obtained from a Multi Variate Model,
b) calculating a likelihood as to whether the sample is from a normal breast tissue, infiltrating ductal carcinoma (IDC) or a benign breast tumor.

In the present context the wording “Multi Variate Model” is to be understood as models defined in terms of several (more than one) parameters.

In a specific embodiment the multivariate model used is Principle Component Analysis (PCA). It is a mathematical algorithm which reduces the dimensionality of the data while retaining most of the variation in the data set. It accomplishes this reduction by identifying directions called principle components along which the variation in the data is maximum. By using a few components each sample can be represented by relatively few numbers instead of by values for thousands of variables. By assisting in determining whether the sample is a normal breast tissue, infiltrating ductal carcinoma (IDC) or a benign breast tumor, a better therapy, diagnosis and prognosis may be obtained. By having a decision supported by multiple methylation patterns a stronger correlation may be obtained

### Data Analysis Using Clinical Parameters

The method according to the invention may take further into account the expression level of different proteins. Thus, in yet an embodiment the invention relates to a method, further comprising determining at least one parameter in a sample obtained from said subject, said parameter being the expression level of at least one of the following proteins selected from the group consisting of Estrogen Receptor (ER), Progesterone receptor (PR) and Herceptin (HER2) in said sample. The person skilled in the art would know that such expression may be determined at e.g. the protein level and/or the RNA level.

By combining both protein expression and methylation status a stronger probability for making correct classification is obtained.

### HER2 Status

To determine which sequences are relevant based on expression levels is not obvious. Thus, in an embodiment the invention relates to a method for assisting in the determining whether a sample is an infiltrating ductal carcinoma or a normal sample,

wherein the HER2 status is determined in a sample, and

wherein the methylation status is determined for at least LRRC4C, HSPA2, ROBO3, AF271776, DFNB31, PGD ((SEQ ID NO: 93, 94, 95, 100, 96, and 97).

Example 7 illustrates how these specific sequences were determined The above sequences had a Fold change (FC) of >1.25 with respect to Her2 status in IDCvsNormal experiments. Fold Change experiments measure the ratio of methylation levels between the case and control (Her2− against Her2+) that are outside of a given cutoff or threshold. The fold change value is the absolute ratio of normalized intensities between the average intensities of all the samples in each group.

From Example 7 it can be seen that SEQ ID NO 93 and 94 which are close to the genes: LRRC4C HSPA2 are likely to be more methylated in Her2+ compared to Her2− in IDC vs. normal differentially methylated samples, while SEQ ID NO 95, 100, 96, and 97 which are close to genes ROBO3, AF271776, DFNB31 and PGD are likely to be less methylated in an IDC sample than in a Normal sample when the sample is HER2+.

### ER Status

Similar as for Her2, specific sequences are found to be particular relevant when the ER status is also known. Thus in yet an embodiment the invention relates to a method for assisting in determining whether a sample is an infiltrating ductal carcinoma or a normal sample,

wherein in the ER status is determined in a sample, and

wherein the methylation status is determined for at least LRRC4C, KIAA0776, NME6, SMG6, ABCB10, MMP25 and LNPEP (SEQ. ID NO: 93, 87, 88, 89, 90, 91 and 92).

Example 5 illustrates how these specific sequences were determined

The above list shows significant loci with fold change >2 in ER+ vs ER− samples of IDCvsNormal

From Example 5 it can be seen that SEQ ID NO 93, 87 (LRRC4C, KIAA0776) are likely to be more methylated in an IDC sample than in a Normal sample and that SEQ ID NO 88, 89, 90, 91 and 92 (NME6, SMG6, ABCB10, MMP25 and LNPEP) are likely to be less methylated in an IDC sample than in a Normal sample when the sample is ER+.

### Menopausal Status

For classifying the samples according to the invention, the menopausal status of the subject from which the sample was obtained may be important. In addition DNA sequences which may be important for determining when the menopausal status is known may also be important. Thus in yet an embodiment the invention relates to a method, for assisting in the determining whether a sample is an infiltrating ductal carcinoma or a normal sample,

wherein in the menopausal status of said subject is determined, and

wherein the methylation status is determined for at least TMEM117, GALNT13, BDNF, and DUSP4 [SEQ ID NO 83, 84, 85, 86].

Example 3 illustrates how said sequences are determined

From Example 3 it can be seen that in IDC vs. normal samples SEQ ID NO 83, 84, and 85 TMEM117, GALNT13 BDNF are likely to be more methylated in postmenopausal sample and that SEQ ID NO 86 DUSP4 are more likely to be methylated in premenopausal sample.

### Combination of ER Status, the PR Status and the HER2

Triple negatives and triple positives are clinically important parameters to judge the efficacy of treatment. Generally triple negatives have poor prognosis and very low survival rate. Again when such triple negatives or positives are determined the classification may be further determined by knowing specific relevant methylation patterns. Thus, in another embodiment the invention relates to a method for assisting in determining whether a sample is an infiltrating ductal carcinoma or a normal sample,

wherein the ER status, the PR status and the HER2 status is determined in a sample, and

wherein the methylation status is determined for LRRC4C, PVRL3, ROBO3, AF271776 SMG6, ABCB10, PVRL3, ROBO3, AF271776, SMG6, AF271776, ABCB10 (SEQ ID NO, 93, 98, 99, 100, 101, 102, 103, and 90). Example 8 illustrates significant loci (FC>1.5) in ER+/PR+/Her2+ against ER−/PR−/Her2− in IDCvsNormal experiments.

From Example 8 it can be seen that the SEQ ID NO 93 which is close to gene LRRC4C has shown higher methylation status in ER+, PR+, Her2+ patients compared to ER−, PR− Her2− samples while Seq ID NO 98, 95, 100, 89, 90 which is close to genes: PVRL3, ROBO3 AF271776, SMG6, and ABCB10 has shown higher methylation status in ER−, PR−, Her2− patients compared to ER+, PR+ Her2+ tumor vs normal samples.

### Infiltrating Ductal Carcinoma or Benign Breast Cancer Tumor

The methods of the invention may also be used for determining whether a sample is a infiltrating ductal carcinoma or benign breast cancer tumor without the use of data on protein expressions. Thus, in an embodiment the invention relates to a method for assisting in the determining whether the sample is from a infiltrating ductal carcinoma or benign breast cancer tumor, wherein the methylation status is determined for at least IFT88, SLC13A3, IREB2, RTTN, KIAA1530, PSIP1, CR601508, BANK1, JAK2 (SEQ ID NO: 104, 105, 106, 107, 108, 109, 110, 111 and 112 respectively).

In example 1 and Table 4 T-test results IDC vs. benign with fold change above 1.5 is shown.

From Example 1 (table 4) it can be seen that SEQ ID NO 102, 105, 107, 110 and 111 corresponding to IFT88, IREB2, KIAA1530, BANK1, JAK2 are likely to be more methylated in an IDC sample than in a benign breast cancer tumor and that SEQ ID NO 104, 106, 108, 109 which correspond to SLC13A3, RTTN, PSIP1 and CR601508 are likely to be less methylated in an IDC sample than in a benign breast cancer tumor.

Invasive Ductal Carcinoma Vs. Normal

The methods of the invention may also be used for determining whether a sample is a infiltrating ductal carcinoma or normal without the use of data on protein expressions. Thus, in an embodiment the invention relates to a method for assisting in the determining whether a sample is an invasive ductal carcinoma or normal, wherein the methylation status is determined for at least ddb1 (SEQ ID NO: 4), DDB1 (SEQ ID NO: 44), DAP (SEQ ID NO:14), TBX3 (SEQ ID NO:29), LRP5 (SEQ ID NO:19) and PCGF2 (SEQ ID NO:24).

We consider five loci which may be very important in distinguishing invasive ductal carcinoma vs. normal: DDB1, DAP and TBX3 (hypermethylated) and LRP5 and PCGF2 (hypomethylated).

SEQ ID NO 4, 44, 14, 29 are likely to be more methylated in an IDC sample than in a normal sample and SEQ ID NO 19 and 24 are likely to be less methylated in an IDC sample than in a normal sample.

By using an even higher number of data points an even more reliable classification may be obtained. Thus, in yet a further embodiment the invention relates to a method for assisting in determining whether a sample is an invasive ductal carcinoma or a normal sample, wherein the methylation is determined for at least 10 sequences selected from the group consisting of: SEQ ID NO: 15 (DUS4L), 27 (SLC17A5), 21 (NR4A2), 20 (NCKIPSD), 57 (PARK2), 2 (CYP26A1), 44(DDB1), 58(PDE4DIP), 14(DAP), 29 (TBX3), 19 (LRP5), 16 (GULP1), 64 (TJP1), 25 (PDE6A), 67 (ZCSL2), 22 (NUP93), 12 (CR596143), 24 (PCGF2), 3 (SNRPF), 18 (L0051057), and 8 (C10orf11). SEQ ID NO. 27, 21, 20, 57, 2, 44, 53, 58, 23, 14, 1, 30, 5, 13, 68, 11, 28, 17, 62, 42, 36, 50, 35, 58, 59, 32, 29, 69, 38, 37, 49, 54, 31, 56, 40, 61, 48, 43, 46, 26, 41, 55, (corresponding to genes: DUS4L, SLC17A5, NR4A2, NCKIPSD, DKFZp7621137, CYP26A1, DDB1, LOC440925, PDE4DIP, OTX1, DAP, BDNF, TRUB2, AB032945, CYP39A1, ZDHHC20, CEP350, SMARCA2, HADHA, SYK, CHD2, ANKHD1, GADD45A, ALG2, PDE4DIP, POLI, ACBD3, TBX3, ZHX2, APOLD1, ANKMY2, FLYWCH1, MALT1, UCK2

NPY1R, BC040897, SIX3, FLRT2, CPEB1, FAM70B, RBPMS2, C6orf155 MORC2) are likely to be more methylated in an IDC sample than in a normal sample and SEQ ID NO 9, 34, 7, 51, 47, 63, 65, 66, 52, 19, 6, 33, 16, 64, 25, 67, 22, 12, 24, 3, 18, 8 (corresponding to genes: PSMB7, C1QTNF8, C17orf41, BC005991, GPR89A, FBXL10, TES, TNFRSF13B, TTC23, HAND2, LRP5, ASNSD1, ACSL3, GULP1, TJP1, PDE6A, ZCSL2, NUP93, CR596143, PCGF2, SNRPF, L0051057, C10orf11) are likely to be less methylated in an IDC sample than in a normal sample.

### Pathways

Thus, in yet an embodiment the invention relates to a method for assisting in determining whether a sample is an invasive ductal carcinoma or a normal sample, wherein the methylation status is determined for at least PCNA, CCND1 MAPK1, SYK (SEQ ID NO 71, 72, 73, 74, 62), BCL2L1, ERBB4 and PARK2 (SEQ ID NO 73,78,79-82, 57), ETS1 and AHR (SEQ ID NO: 75, 76).

SEQ ID NO 73, 74, 62, 57, 78 are likely to be more methylated in an IDC sample than in a normal sample and SEQ ID NO 71, 72, 75, 76, 79, 80, 81, 82 are likely to be less methylated in an IDC sample than in a normal sample.

### Determination of Methylation Status

The methylation status of a sample may be determined by different means. Thus, in an embodiment the methylation status is determined by means of one or more of the methods selected form the group of,

a. bisulfite sequencing

b. pyrosequencing

c. methylation-sensitive single-strand conformation analysis(MS-SSCA)

d. high resolution melting analysis (HRM)

e. methylation-sensitive single nucleotide primer extension (MS-SnuPE)

f. base-specific cleavage/MALDI-TOF

g. methylation-specific PCR (MSP)

h. microarray-based methods and

i. msp I cleavage.

j. Methylation sensitive sequencing

In addition to the described method in our patent disclosure, there is a variety of methods for determining the methylation status of a DNA molecule. It is preferred that the methylation status is determined by means of one or more of the methods selected form the group of, 10arkinson sequencing, methylation-sensitive single-strand conformation analysis(MS-SSCA), high resolution melting analysis (HRM), methylation-sensitive single nucleotide primer extension (MS-SnuPE), base-specific cleavage/MALDI-TOF, methylation-specific PCR (MSP), methyl-binding protein immunoprecipitation, microarray-based methods, enzymatic assays involving McrBc and other enzymes such as Msp I. An overview of the known methods of detecting 5-methylcytosine may be found from the following review paper: Rein, T., DePamphilis, M. L., Zorbas, H., Nucleic Acids Res. 1998, 26, 2255. Further methods are disclosed in US 2006/0292564A1.

### Sample Type

The samples according to the invention may be obtained from different types of sample material. Thus, in an embodiment the sample to be analyzed is from a tissue type selected from the group of tissues such as, a tissue biopsy from the tissue to be analyzed, tumor tissue, body fluids, blood, serum, saliva and urine. In a specific embodiment the sample is tissue biopsy such as a breast tissue biopsy. In another embodiment the sample is provided from a human, more specifically the subject is a female.

### Prediction of the Therapeutic Response

The methods according to the invention may also be used for evaluate the efficiency of a treatment. Thus in an embodiment the methylation pattern obtained, is used to predict the therapeutic response to the treatment of a breast cancer. This may be done by measuring the methylation pattern before or after a treatment is initiated or during a treatment. Thus, it may be possible to determine whether the subject receives correct treatment.

### Composition or Array

The present invention also relates to composition or arrays comprising 10 or more sequences according to the invention. Thus, in an aspect the invention relates to a composition or array comprising nucleic acids with sequences which are identical to at least 10 of the sequences according to SEQ ID NO: 1-111. Similar, in an embodiment the invention relates to a composition or arrays comprising nucleic acids with sequences which are identical to at least 20, such as at least 40 such as at least 60 of the sequences according to SEQ ID NO: 1-111.

It is of course also to be understood that the composition or array may comprise at least one or more of the specific subset of sequences listed in tables and claims.

In another embodiment the invention relates to a composition or array, comprising nucleic acids with sequences which are identical to ddb1 (SEQ ID NO:4), DDB 1 (SEQ ID NO 44), DAP (SEQ ID NO:14), TBX3 (SEQ ID NO:29), LRP5 (SEQ ID NO:19) and PCGF2 (SEQ ID NO:24).

### Computer Program

The methods according to the invention may also be performed by a computer program. Thus, in an aspect the invention relates to a computer program product being adapted to enable a computer system comprising at least one computer having a data storage means associated therewith to operate a processor arranged for carrying out a method according to the invention.

### EXAMPLES

### Example 1

### Description of the CpG Island Arrays

The CpG arrays used in our experiments are special ordered arrays, offered by University Health Network Microarray centre, Toronto, Canada. Each array consists of 12192 spotted clones. All clones were sequenced originally at Sanger, with further verification performed at the British Columbia Genome Sciences Centre and internally at the UHN Microarray Centre. The library was made by cutting genomic DNA with Msel enzyme, which cuts at AATT points. Methylated fragments, i.e. those that are not being protected and therefore probably not a CpG island, are then pulled out on a column and discarded. The remaining fragments are artificially methylated and then this is run through a column which pulls out those methylated fragments which represent CpG islands. These DNA segments are then cloned into vectors, grown on plates, picked, amplified and spotted onto the array.

Here is a summary of the clones on the array: there is an annotation file Cpgdump which provides information such as the genomic location of each clone, its sequence, overlapping transcript IDs, nearest upstream and downstream transcript IDs and so forth
No. of Clones for which Sequence is present: 11539
No. of clones with Forward sequence—10216
No. of clones with Reverse Sequence—10458
Number of clones that are associated with a gene: 5530. This means that the clone is either in the promoter region of a gene (less than a 2000 base pairs of a transcription start site), within the boundaries of a gene, or up to 2000 bases downstream of the 3′ end of the gene.
Max. length of Sequence—991
Average Length of Sequence—326.19

### Experimental Protocol for Array Hybridization

At the time of surgery one sample of fresh tissue and another in 10% formalin were collected. Fresh frozen tissue is used for subsequent DNA extraction and hybridization experiments. The sample collected in 10% formalin is processed to make a formalin fixed paraffin embedded block for histopathological and hormone receptor studies. Slides from these blocks were stained with Hematoxylin & Eosin and reviewed by pathologists for classification and grading of tumors. Immumunohistochemistry for ER, PR, HER2, was done on each set of formalin-fixed, paraffin-embedded tissue slides using the primary antibodies from DAKO and secondary as Envision™ method with 3, 3diaminobenzidine chromogen. Biomarker expression from immunohistochemical assays were scored independently by two pathologists, using previously established scoring methods. ER and PR stains were considered positive if immune-staining was seen in >1% of tumor nuclei. For HER2 status, tumors were considered positive if scored as 3+ according to HercepTest™ criteria.

The following steps are performed by the hybridization protocol:

1. Collect Sample

2. Extract DNA (24 hrs)

3. Check for Concentration and quality (4 hrs)

4. Digest with Msel (16 hrs)

5. Purify and Precipitate (24 hrs)

6. Check Concentration (4 hrs)

7. Anneal Primers (14 hrs)

8. Ligate to DNA (24 hrs)

9. Perform PCRs (qualitative and quantitative (24 to 7 hrs)

10. Purify DNA (24 hrs)

11. Label with Dyes (24 hrs)

12. Check for labelling (2 hrs)

13. Purify DNA and quantify (24 hrs)

14. Hybridize to Chips

### Clinical Data Description

The prospective study cohort consists of 51 female primary breast cancers. All patients had been undergoing treatment in a tertiary care hospital and its associated centres in Southern part of India between 2007 and 2009. Information pertaining to age, menopausal status, staging, histopathological type, hormonal receptor status of the patients was collected after patient consent and ethical committee approval. Limited follow-up data was available considering the first sample collection was only 2 years ago and extrapolating this information to outcomes is not justified. The study cohort underwent mastectomy with or without chemo and radio therapy.

The description of the clinical data being used is given in Table 1. The data classification has been derived after extensive discussions with multiple clinical experts. The two major categories in this sample set were IDC vs Normal and IDC vs Benign with 29 and 16 samples respectively in each category. The other categories had fewer samples and were not included for further analysis. The type of experiments for which further analysis was conducted is: infiltrating ductal carcinoma (IDC) vs. Normal and infiltrating ductal carcinoma (IDC) vs. benign condition.

In the present context “infiltrating ductal carcinoma (IDC) vs. Normal” refers to a ratio between the differential methylation status of genes present among the infiltrating ductal carcinoma (IDC) samples as well as the normal samples. Similar, in the present context the term “infiltrating ductal carcinoma (IDC) vs. benign condition” is to be understood as the differentially methylated genes among IDC samples and benign tumor samples. This comparison is of importance as the benign tumor samples are seen as being potentially premalignant.

TABLE 1
Clinical sample classification used in the data analysis.

| | | Menopausal | | | | | | ER+ | ER− | | |
|---|---|---|---|---|---|---|---|---|---|---|---|
| | | status | | | Onset | | | PR+ | PR− | Size | |
| Category | Total | Pre | Post | NA | Early | Mid | Late | Her2+ | Her2− | <5 cm | >5 cm |
| IDC vs | 29 | 9 | 10 | 10 | 9 | 9 | 11 | 11 | 5 | 8 | 21 |
| Normal | | | | | | | | | | | |
| IDC vs | 16 | 4 | 0 | 12 | 2 | 14 | 0 | 5 | 4 | 5 | 8 |
| Benign | | | | | | | | | | | |

### Data Analysis of Carcinoma, Normal and Benign Conditions

The experiments were conducted as paired samples of normal samples with cancer samples. As far as possible adjacent normal of the cancer sample was used. Some cases benign tumors were paired with malignant samples. Benign tumors included fibroadenoma, fibrocystic disease, adenosis and phyllodes tumour.

After the hybridization step, the microarray chips are scanned and the intensity values across the chip recorded. The proprietary feature extraction software from Agilent executes the basic image processing algorithms to quantify the intensity values at each spot while correcting for the background noise. At the end of this process, a QC report is prepared and a matrix of raw values is exported which includes the raw and minimally normalized intensity values for each gene/locus in the array.

The first step in data analysis is to carry out further normalization of the matrix data to account for intra-array and inter-array experimental deviations. The raw values at each matrix are normalized to an upper limit of 1.0 over a log scale and normalized using LOWESS (locally weighted scatter plot smoothing) method.

### Pre-Processing Based on Carcinoma Subtype Classification

I. All 45 ductal carcinoma arrays were normalized prior to determining the differential gene expression between normal and ductal carcinoma samples using LOWESS method.
II. Interarray normalization is performed in several different methods: baseline to median (in GeneSpring GX 10), normalize mean to zero, and quantile normalization (in R/Bioconductor).
III. Correlation assessment among all the experiments is then computed to get a picture of the similarity in the array data among the samples in the set.

We used R/Bioconductor and GeneSpring v10 for statistical analysis of the breast cancer data.

IDC Vs. Normal Statistical Analysis with Outer Loop Validation

We also performed analysis using only the promoter probes (modified files) which gives 71 significant loci in total. Here is a table with all the probes that actually have “survived” the following steps:
1. The raw matrix is taken from the corrected signal where features are extracted (normalized) using only 5530 probes—not all probes.
2. Further, the obtained microarray data is preprocessed with Lowess intra-array normalization
3. Quantile inter-array normalization is performed on MA matrix. For further processing M is used. (log ratio)
4. Fold change is greater than 0.7 (or less than −0.7) in at least 14 out of the 29 IDC vs. normal samples
5. The p-value is less than 0.05 in a leave one out procedure (29 repeats where one sample is left out from the t-test). The final result table has 71 UHN ids (with gene symbols included).
6. With the adjusted p-values obtained from the Bayesian statistical analysis also in a leave one out fashion, we exclude 7 probes, which leave 64 probes as the final result.

Results are shown in Table 3. It is important to note that these loci are obtained with a leave one out validation and should be more stable and less sensitive to noise. The p-values shown in the table are obtained using all samples. Also, due to the Quantile normalization, the values of around 1 should be considered extremely high. In Table 15, we present the most significant of these loci with SEQ ID: 15, 27, 21, 20, 57, 2, 44, 58, 14, 29, 19, 16, 64, 25, 67, 22, 12, 24, 3, 18, and 8, which correspond to genes: DUS4L, SLC17A5, NR4A2, NCKIPSD, PARK2, CYP26A1, DDB1, PDE4DIP, DAP, TBX3, LRP5, GULP1, TJP1, PDE6A, ZCSL2, NUP93, CR596143, PCGF2.

TABLE 3
Results of IDC vs. normal t-testing from a leave one out validation
loop.

| SEQ ID | | | Adjusted | |
|---|---|---|---|---|
| NO | ID | Gene symbol | p-value | Mean |
| 68 | UHNhscpg0007132 | ZDHHC20 | 4.87E−05 | 0.822711 |
| 1 | UHNhscpg0003204 | BDNF | 4.87E−05 | 0.87014 |
| 21 | UHNhscpg0006767 | NR4A2 | 6.90E−05 | 1.033697 |
| 20 | UHNhscpg0009447 | NCKIPSD | 0.000101 | 1.011746 |
| 57 | UHNhscpg0008659 | PARK2 | 0.00015 | 1.002518 |
| 14 | UHNhscpg0005129 | DAP | 0.0002 | 0.881149 |
| 36 | UHNhscpg0003749 | ANKHD1 | 0.000238 | 0.797185 |
| 32 | UHNhscpg0006074 | ACBD3 | 0.000292 | 0.759773 |
| 53 | UHNhscpg0010276 | LOC440925 | 0.000335 | 0.927716 |
| 8 | UHNhscpg0005168 | C10orf11 | 0.000403 | −1.11219 |
| 15 | UHNhscpg0004955 | DUS4L | 0.000462 | 1.202454 |
| 11 | UHNhscpg0007121 | CEP350 | 0.000496 | 0.822555 |
| 38 | UHNhscpg0001556 | APOLD1 | 0.000516 | 0.749436 |
| 58 | UHNhscpg0007517 | PDE4DIP | 0.000528 | 0.905226 |
| 62 | UHNhscpg0004894 | SYK | 0.00053 | 0.810273 |
| 2 | UHNhscpg0000746 | CYP26A1 | 0.000555 | 0.934528 |
| 70 | UHNhscpg0003020 | DKFZp762I137 | 0.000555 | 0.946523 |
| 27 | UHNhscpg0006718 | SLC17A5 | 0.000693 | 1.076886 |
| 49 | UHNhscpg0007607 | FLYWCH1 | 0.000796 | 0.742613 |
| 40 | UHNhscpg0006298 | BC040897 | 0.000915 | 0.683741 |
| 29 | UHNhscpg0006737 | TBX3 | 0.001042 | 0.754758 |
| 17 | UHNhscpg0011146 | HADHA | 0.001147 | 0.810381 |
| 44 | UHNhscpg0008660 | DDB1 | 0.001158 | 0.928127 |
| 50 | UHNhscpg0007178 | GADD45A | 0.001258 | 0.79172 |
| 13 | UHNhscpg0007485 | CYP39A1 | 0.001296 | 0.850419 |
| 23 | UHNhscpg0002087 | OTX1 | 0.001316 | 0.889817 |
| 5 | UHNhscpg0007521 | AB032945 | 0.001624 | 0.856789 |
| 59 | UHNhscpg0007487 | POLI | 0.001624 | 0.770442 |
| 35 | UHNhscpg0008517 | ALG2 | 0.001708 | 0.785926 |
| 10 | UHNhscpg0007200 | FLJ10996 | 0.001999 | 0.771389 |
| 31 | UHNhscpg0008746 | UCK2 | 0.001999 | 0.714308 |
| 6 | UHNhscpg0005119 | ASNSD1 | 0.002328 | −0.6714 |
| 9 | UHNhscpg0003195 | C1QTNF8 | 0.002422 | −0.5403 |
| 43 | UHNhscpg0007469 | CPEB1 | 0.002422 | 0.637375 |
| 16 | UHNhscpg0000358 | GULP1 | 0.002478 | −0.7189 |
| 67 | UHNhscpg0000299 | ZCSL2 | 0.002814 | −0.84025 |
| 22 | UHNhscpg0000109 | NUP93 | 0.002828 | −0.87988 |
| 69 | UHNhscpg0007446 | ZHX2 | 0.003114 | 0.750184 |
| 42 | UHNhscpg0009610 | CHD2 | 0.003212 | 0.800779 |
| 60 | UHNhscpg0009180 | PSMB7 | 0.003593 | −0.43153 |
| 3 | UHNhscpg0000390 | SNRPF | 0.00439 | −1.00775 |
| 37 | UHNhscpg0001513 | ANKMY2 | 0.004468 | 0.743584 |
| 58 | UHNhscpg0007602 | PDE4DIP | 0.00455 | 0.777924 |
| 41 | UHNhscpg0006075 | C6orf155 | 0.005387 | 0.505702 |
| 4 | UHNhscpg0003291 | SULF1 | 0.005914 | 0.684412 |
| 18 | UHNhscpg0000591 | LOC51057 | 0.006152 | −1.02894 |
| 28 | UHNhscpg0007553 | SMARCA2 | 0.006152 | 0.814892 |
| 54 | UHNhscpg0005089 | MALT1 | 0.006747 | 0.729116 |
| 61 | UHNhscpg0003180 | SIX3 | 0.006956 | 0.666075 |
| 12 | UHNhscpg0000322 | CR596143 | 0.007368 | −0.93453 |
| 30 | UHNhscpg0005296 | TRUB2 | 0.008113 | 0.857046 |
| 56 | UHNhscpg0007104 | NPY1R | 0.010879 | 0.70281 |
| 19 | UHNhscpg0000038 | LRP5 | 0.013234 | −0.66959 |
| 24 | UHNhscpg0000193 | PCGF2 | 0.015044 | −0.99558 |
| 26 | UHNhscpg0004952 | RBPMS2 | 0.016904 | 0.519043 |
| 45 | UHNhscpg0007159 | MGC23280 | 0.018887 | 0.765995 |
| 34 | UHNhscpg0000043 | AKT1S1 | 0.021285 | −0.63249 |
| 63 | UHNhscpg0000364 | TES | 0.021557 | −0.64469 |
| 51 | UHNhscpg0000037 | GPR89A | 0.025007 | −0.64381 |
| 48 | UHNhscpg0000429 | FLRT2 | 0.027045 | 0.642276 |
| 25 | UHNhscpg0005166 | PDE6A | 0.028382 | −0.74392 |
| 55 | UHNhscpg0007662 | MORC2 | 0.033752 | 0.487627 |
| 46 | UHNhscpg0000452 | FAM70B | 0.043458 | 0.565759 |
| 7 | UHNhscpg0005159 | BC005991 | 0.048081 | −0.64101 |

IDC Vs. Benign Statistical Analysis

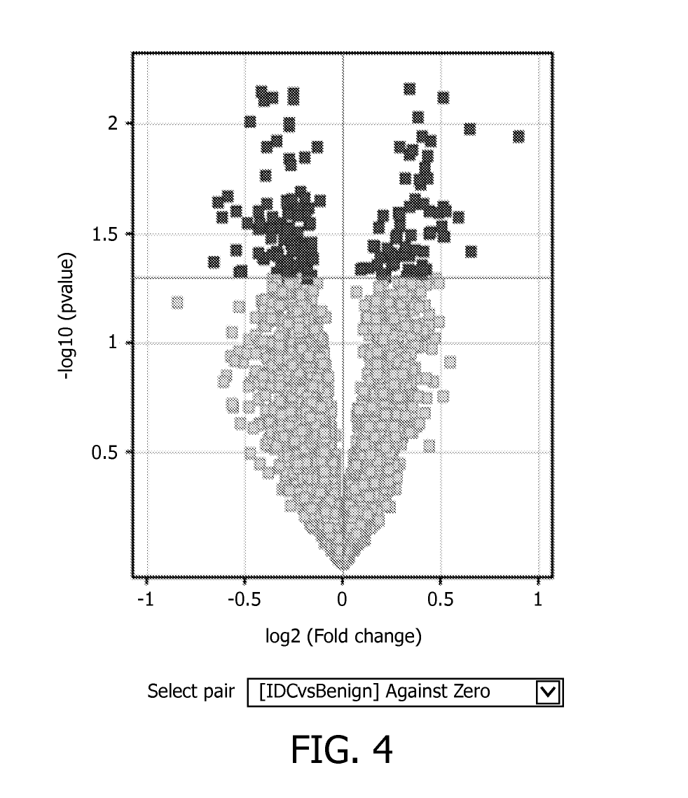

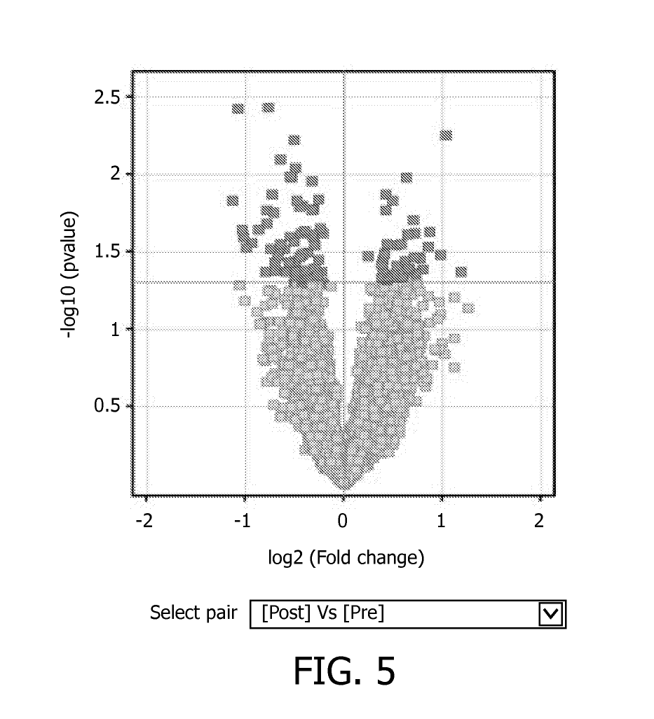

Using GeneSpring 10, we performed T-test against zero-mean hypothesis on the IDC vs. benign experiments. We used total of 16 experiments and performed t-test without multiple testing correction and obtained 160 significant loci. Out of that, we have 155 entities with fold change greater or equal to 1.1. The significant differentially methylation loci between IDC vs. benign are shown in Table 4. Volcano plot is shown in FIG. 4. Differentially methylated sequences are close to genes: IFT88, SLC13A3, IREB2, RTTN, KIAA1530, PSIP1, CR601508, BANK1, JAK2 (SEQ ID NO: 103, 104, 105, 106, 107, 108, 109, 110, 111 respectively). The sequences 102, 105, 107, 110 and 111 corresponding to IFT88, IREB2, KIAA1530, BANK1, JAK2 are methylated more in IDC than in benign tumor while sequence numbers: 104, 106, 108, 109 which correspond to SLC13A3, RTTN, PSIP1 and CR601508 are methylated more in benign than in IDC samples.

TABLE 4
T-test results IDC vs. benign with fold change above 1.5.

| SEQ | | | | | |
|---|---|---|---|---|---|
| ID | | Fold | | Gene | |
| NO | UHNID | Change | Change | symbol | Description |
| 103 | UHNhscpg0007777 | 1.5708911 | up | IFT88 | intraflagellar transport 88 |
| | | | | | homolog isoform 1 |
| 104 | UHNhscpg0000501 | 1.5785927 | down | SLC13A3 | solute carrier family 13 |
| | | | | | member 3 isoform a |
| 105 | UHNhscpg0007046 | 1.8579512 | up | IREB2 | Iron responsive element |
| | | | | | binding protein 2 |
| 106 | UHNhscpg0008329 | 1.5022352 | down | RTTN | rotatin |
| 107 | UHNhscpg0000211 | 1.5032853 | up | KIAA1530 | KIAA1530 protein |
| 108 | UHNhscpg0002300 | 1.5540606 | down | PSIP1 | PC4 and SFRS1 |
| | | | | | interacting protein 1 |
| | | | | | isoform 2 |
| 109 | UHNhscpg0004523 | 1.5321043 | down | CR601508 | OTTHUMP00000016614. |
| 110 | UHNhscpg0009237 | 1.6035372 | up | BANK1 | Hypothetical protein |
| | | | | | FLJ34204. |
| 111 | UHNhscpg0006618 | 1.5664941 | Up | JAK2 | Janus kinase 2 |

### Example 2

### Data Analysis Using Clinical Parameters

It is very important for clinical decision making to more accurately decide if a patient has differentially methylated loci that correspond more to the IDC vs. normal based on the menopausal status or based on the onset of the disease which could be early or late.
I. Out of 29 samples of infiltrating ductal carcinoma that were matched with normals for experimentation, 9 were found to be in premenopausal women and 10 were in post-menopausal women.
II. The two sub groups were defined as a particular interpretation. All entities that passed the student's t test with a confidence of 99.95% were first selected.
III. Fold Change Analysis is used to identify genes with expression ratios or differences between a treatment and a control that are outside of a given cut-off or threshold. Fold change gives the absolute ratio of normalized intensities (no log scale) between the average intensities of the samples grouped. The results were filtered on fold change >=1.75 and >=2.
IV. The data was also filtered by expression. In this process, all entities that satisfy the top 30 percentile in the normalized data in majority of the samples are selected and verified.

### Example 3

### Menopause Status Based Classification

I. 109 out of 5530 entities were found to be significant when passed through the student t-test (unpaired, asymptotic, no correction).
II. Following fold change on Post vs. Pre Menopausal status of all entities, 4 entities loci were found to be significantly differentiated with a fold change of >=1.3
III. The most significant UHN loci were picked by passing them through a filter for expression of the loci in the top 10 percentile of the data in majority of the samples.

TABLE 6
List of genes with significant changes in methylation between post
menopausal vs. premenopausal tumor patients.

| SEQ | | | | | |
|---|---|---|---|---|---|
| ID | | | | | Gene |
| NO | UHNID | Fold Change | Change | Description | symbol |
| 83 | UHNhscpg0007411 | 1.3591343 | up | hypothetical protein | TMEM117 |
| | | | | LOC84216 | |
| 84 | UHNhscpg0008515 | 1.3944643 | up | UDP-N-acetyl-alpha-D- | GALNT13 |
| | | | | galactosamine:polypeptide | |
| 85 | UHNhscpg0008264 | 1.4317298 | up | brain-derived neurotrophic | BDNF |
| | | | | factor isoform b | |
| 86 | UHNhscpg0002632 | 1.6052125 | down | dual specificity phosphatase | DUSP4 |
| | | | | 4 isoform 1 | |

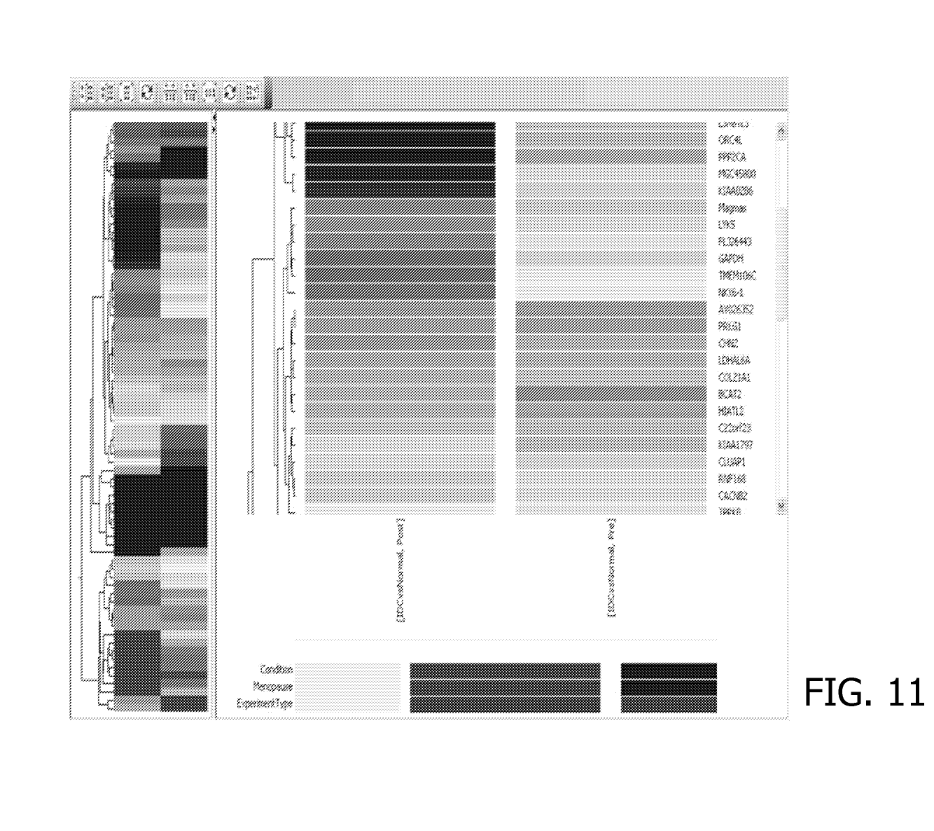

In FIG. 11 Clustering on IDCvsNormal samples after t-test post vs. premenopausal status, p-value cut off <=0.05.

FIG. 7: Fold change of 4 loci between post and pre menopausal cases with a fold change >1.3.

As can be seen from the FIG. 7, SEQ ID NO 83, 84, 85 TMEM117, GALNT13 BDNF and are likely to be more methylated in postmenopausal sample and that SEQ ID NO DUSP4 is more likely to be methylated in premenopausal sample when the methylation status of tumor vs. normal is examined.

### Example 4

### Estrogen Receptor (ER), Progesterone Receptor (PR) and Herceptin (Her2)

Another important set of parameters to consider while screening for differentiators between tumor and normal is the Hormone receptors status. We analysed the presence or absence of Estrogen Receptor (ER), Progesterone Receptor (PR) and Herceptin (Her2) in all the tumor samples. The experiments were classified based on the status of these three parameters and the significant differences in these tumor types were noted.

TABLE 7
Categories of Hormone receptor status

| | ER | PR | Her2 | ER/PR/Her2 |
|---|---|---|---|---|
| Positive | 19 | 16 | 17 | 11 |
| Negative | 8 | 11 | 10 | 5 |

Fold change analysis and clustering was done on the above categories using the significant entities within IDCvsNormal (p<0.05) as the input data set.

### Example 5

### ER Status Based Classification

a. 72 out of 5053 entities were found to be significant when passed through the student t-test for IDCvsNormal (unpaired, asymptotic, no correction).
b. Fold change on ER+ vs ER− status samples classified based on clinical data from patients into ER+ vs. ER− ve for all entities resulted in 6 entities loci which were significantly differentiated with a difference of >=2.0 (listed in table 8)
c. The most significant UHN loci were picked by passing them through a filter for expression of the loci in the top 10 percentile of the data in majority of the samples.
d. Clustering analysis was also done on the significant loci to look for patterns of hyper/hypo methylation across the samples. The results are displayed in FIG. 9

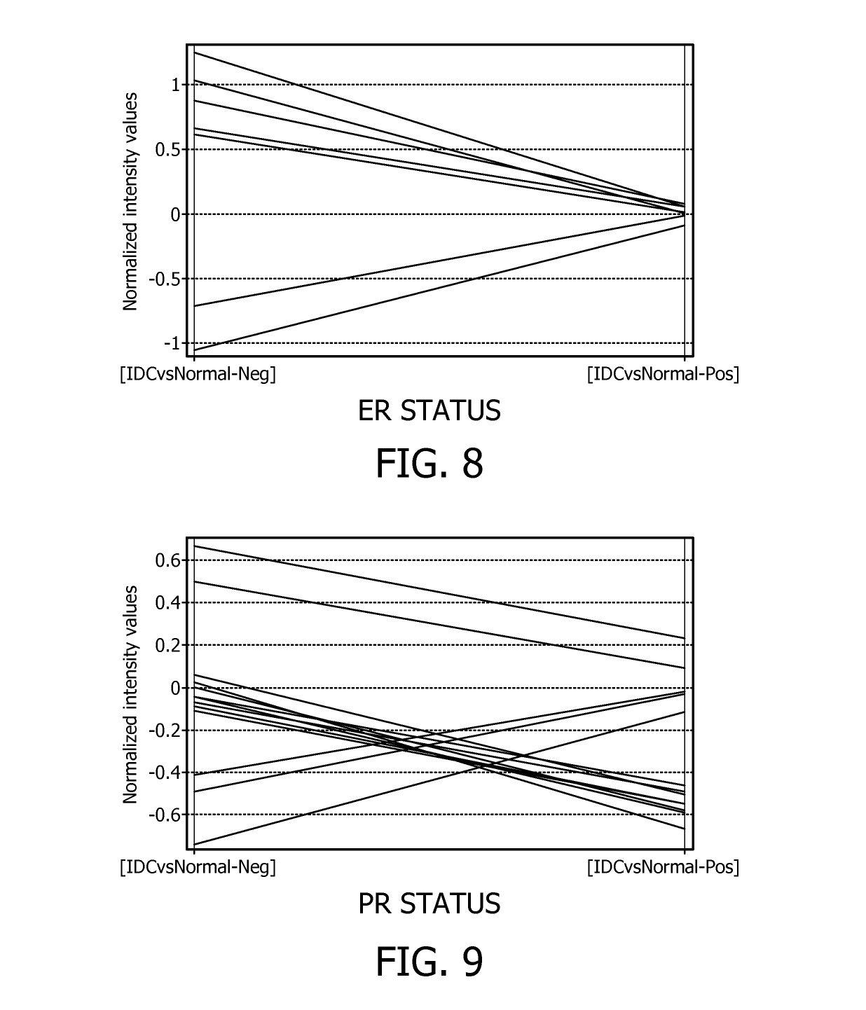

FIG. 8: Fold change of between ER+ against ER− samples

TABLE 8
Significant loci with fold change >2 in ER+ vs ER− samples of
IDC vs Normal

| | | | |
|---|---|---|---|
| SEQ | UHNhscpg0000636 | down | Netrin-G1 ligand |
| ID NO 93 | | | |
| 87 | UHNhscpg0006957 | down | hypothetical protein LOC23376 |
| 88 | UHNhscpg0008950 | up | “non-metastatic cells 6, protein |
| | | | expressed in (nucleoside- |
| | | | diphosphate kinase)” |
| 89 | UHNhscpg0000024 | up | Est1p-like protein A |
| 90 | UHNhscpg0010841 | up | “ATP-binding cassette, |
| | | | sub-family B, member 10” |
| 91 | UHNhscpg0010601 | up | matrix metalloproteinase 25 |
| | | | preproprotein |
| 92 | UHNhscpg0011399 | up | leucyl/cystinyl aminopeptidase |
| | | | isoform 1 |

SEQ ID NO 93 and 87 (LRRC4C and KIAA0776) have higher methylation in ER+ when compared to ER− samples when IDC is compared to normal sample, while SEQ ID NO 88, 89, 90, 91 and 92 have higher methylation status in ER− compared to ER+ samples.

### Example 6

### PR Status Based Classification

a. Fold change on PR+ vs PR− ve [samples classified based on clinical data from patients into] status of all entities resulted in 13 entities loci which were significantly differentiated with a difference of >=2.0 (listed in table 9).
b. The most significant UHN loci were picked by passing them through a filter for expression of the loci in the top 10 percentile of the data in majority of the samples.
c. Clustering analysis reveals the presence of two main classes of groups as shown in FIG. 11.

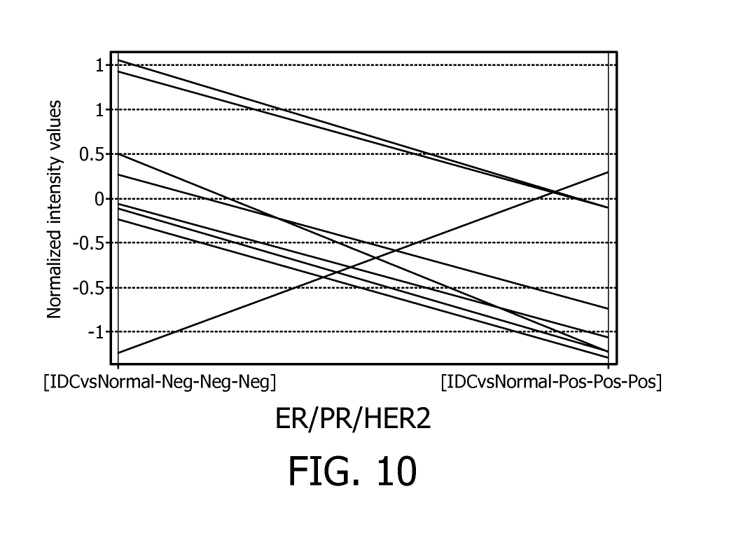

FIG. 10: Fold change of between PR− against PR+ samples

TABLE 9
Significant loci with fold change >2.0 with respect to PR+ against PR−
in IDCvsNormal experiments

| SEQ ID NO | UHNhscpg0004504 | down | Glyceraldehyde-3-phosphate |
|---|---|---|---|
| 999 | | | dehydrogenase(EC1.2.1.12) |
| | | | (Fragment). |
| 93 | UHNhscpg0000636 | down | netrin-G1 ligand |
| 102 | UHNhscpg0000230 | up | distal-less homeobox 6 |
| 98 | UHNhscpg0004672 | up | PVRL3 protein. |
| 87 | UHNhscpg0006957 | down | hypothetical protein |
| | | | LOC23376 |
| 95 | UHNhscpg0001461, | up | “roundabout, axon guidance |
| | UHNhscpg0001274 | | receptor, homolog 3” |
| 100 | UHNhscpg0000914, | up | ATP synthase a chain |
| | UHNhscpg0002255, | | (EC 3.6.3.14) (ATPase |
| | UHNhscpg0002136, | | protein 6). |
| | UHNhscpg0002944 | | |
| 89 | UHNhscpg0000024 | up | Est1p-like protein A |
| 96 | UHNhscpg0005839 | up | OTTHUMP00000021976. |

That SEQ ID NO 99, 93, 87, GAPDH and LRRC4C, KIAA0776 are methylated more in PR+ and SEQ ID NO 102, 98, 95, 100, 89, 96 DLX6, PVRL3, ROBO3, AF271776, SMG6, DFNB31, are methylated more in PR− in differentially methylated tumor vs. Normal samples.

### Example 7

### Her2 Status Based Classification

Fold change on Her2+ vs. Her2− [samples classified based on clinical data from patients into Her2+ and Her2− status of all entities resulted in 6 entities loci which were significantly differentiated with a difference of >=1.25 (listed in table 10).

TABLE 10
Fold change of >1.25 with respect to Her2 status in IDCvsNormal
experiments

| | | | |
|---|---|---|---|
| SEQ ID NO | UHNhscpg0000636 | down | netrin-G1 ligand |
| 93 | | | |
| 94 | UHNhscpg0007219 | down | heat shock 70 kDa protein 2 |
| 95 | UHNhscpg0001461 | Up | “roundabout, axon guidance |
| | | | receptor, homolog 3” |
| 100 | UHNhscpg0000914 | Up | ATP synthase a chain |
| | | | (EC 3.6.3.14) (ATPase |
| | | | protein 6). |
| 96 | UHNhscpg0005839 | Up | OTTHUMP00000021976. |
| 97 | UHNhscpg0010619 | Up | phosphogluconate |
| | | | dehydrogenase |

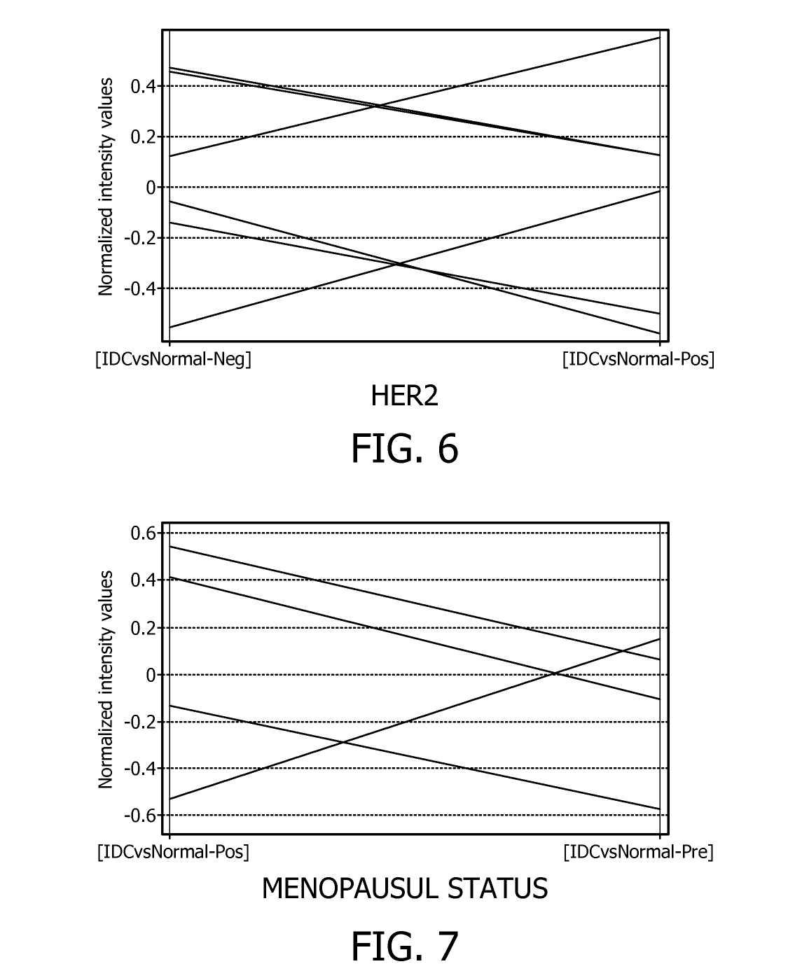

The plot in FIG. 6 shows that the overall ratio of the methylation status changes between IDC and Normal for the above six sequences with respect to the HER2 status.

In conclusion what can be seen in table 10 and FIG. 6 is that for the respective loci: SEQ ID NO 93 and 94 which are close to the genes: LRRC4C HSPA2 is higher in Her2+ compared to Her2− tumor vs. normal differentially methylated samples while SEQ ID NO 95, 100, 96, and 97 which are close to genes ROBO3, AF271776, DFNB31, and PGD methylation is higher in Her2− samples compared to Her2+.

### Example 8

### ER/PR/Her2 Status Based Classification

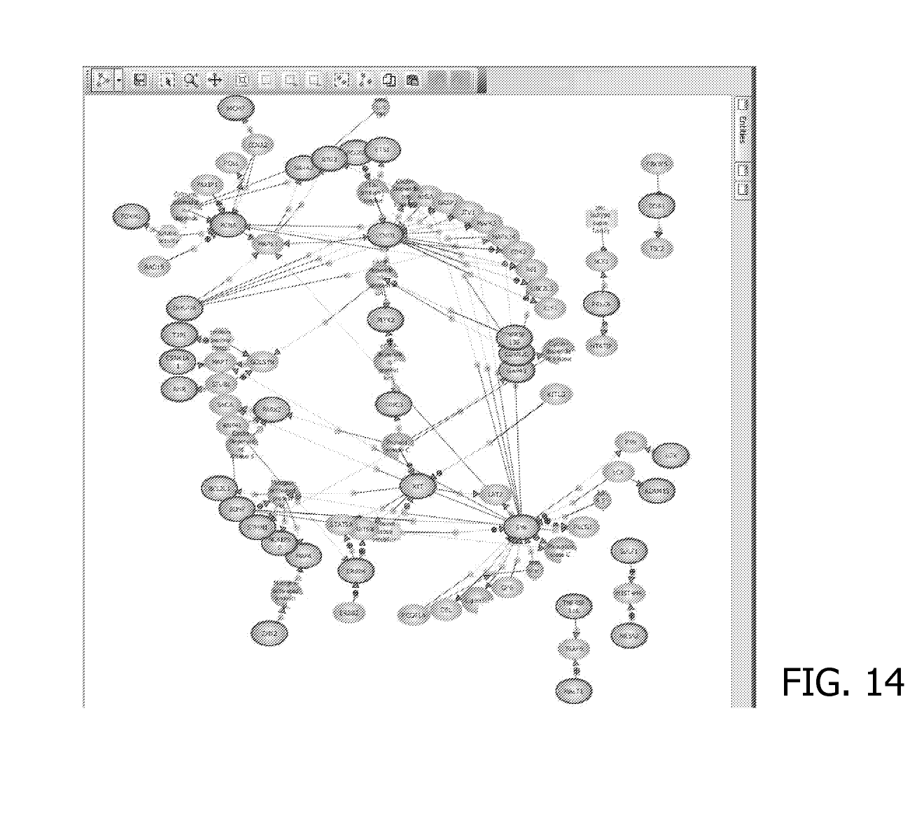

Triple negatives and triple positives are clinically important parameters to judge the efficacy of treatment. Generally triple negatives have poor prognosis and very low survival rate.
I. Fold change on ER, PR, Her2, samples classified based on clinical data from patients into ER+/PR+/Her2+ against ER−/PR−/Her2− status of all entities resulted in 8 entities loci which were significantly differentiated with a difference of >=1.5 (listed in table 11)
II. The most significant UHN loci were picked by passing them through a filter for expression of the loci in the top 10 percentile of the data in majority of the samples.
III. Clustering of the loci with respect to triple positives against triple negatives yielded three clearly distinguishable clusters of genes (FIG. 14).

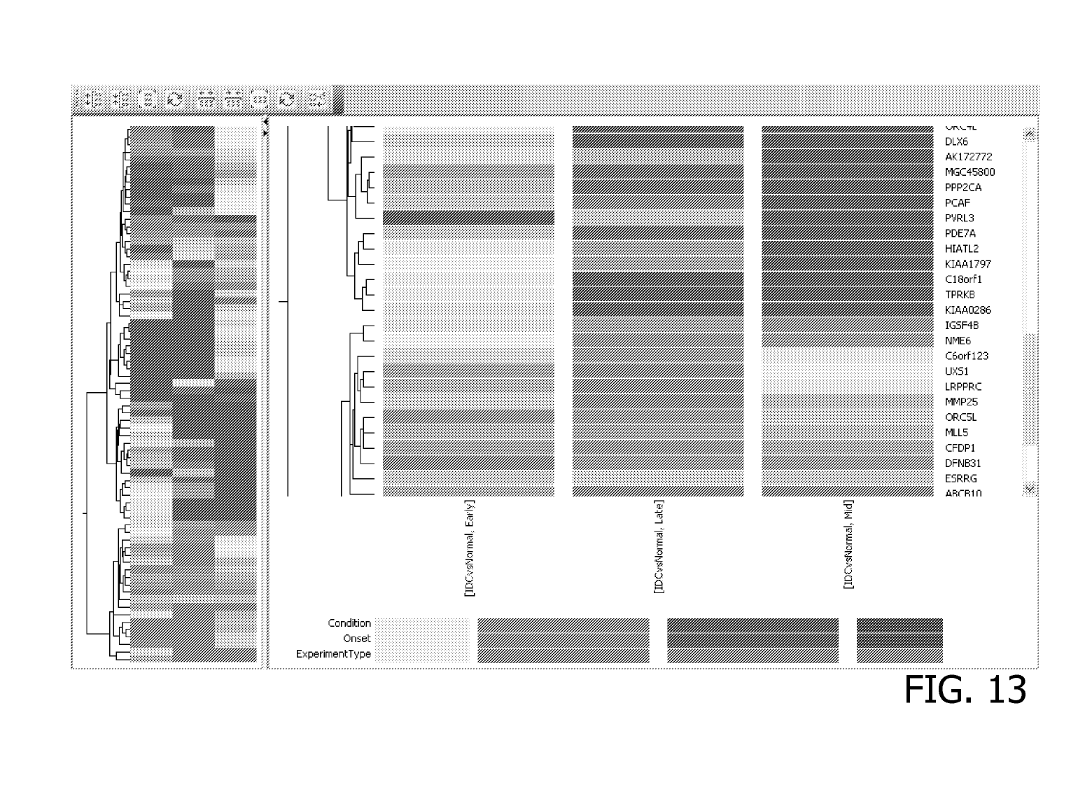

FIG. 13: Fold change of between ER−/PR−/Her2− against ER+/PR+/Her2+ samples.

TABLE 11
Significant loci (FC > 1.5) in ER+/PR+/Her2+ against ER−/PR−/Her2−
in IDCvsNormal experiments.

| | | | |
|---|---|---|---|
| SEQ ID NO | UHNhscpg0000636 | down | netrin-G1 ligand |
| 93 | | | |
| 98 | UHNhscpg0004672 | up | PVRL3 protein. |
| 95 | UHNhscpg0001274 | up | “roundabout, axon guidance |
| | | | receptor, homolog 3” |
| 100 | UHNhscpg0000914, | up | ATP synthase a chain |
| | UHNhscpg0002255, | | (EC 3.6.3.14) (ATPase |
| | UHNhscpg0002136 | | protein 6). |
| 89 | UHNhscpg0000024 | up | Est1p-like protein A |
| 90 | UHNhscpg0010847 | up | “ATP-binding cassette, |
| | | | sub-family B, member 10” |

The SEQ ID NO 93 which is close to gene LRRC4C has shown higher methylation status in ER+, PR+, Her2+ patients compared to ER−, PR− Her2− samples. Whereas SEQ ID NO 98 95 100 89 90 which is close to genes: PVRL3, ROBO3, AF271776 SMG6, ABCB10 has shown higher methylation status in ER−, PR−, Her2− patients compared to ER+, PR+Her2+ tumor vs normal samples.

### Example 9

### Onset

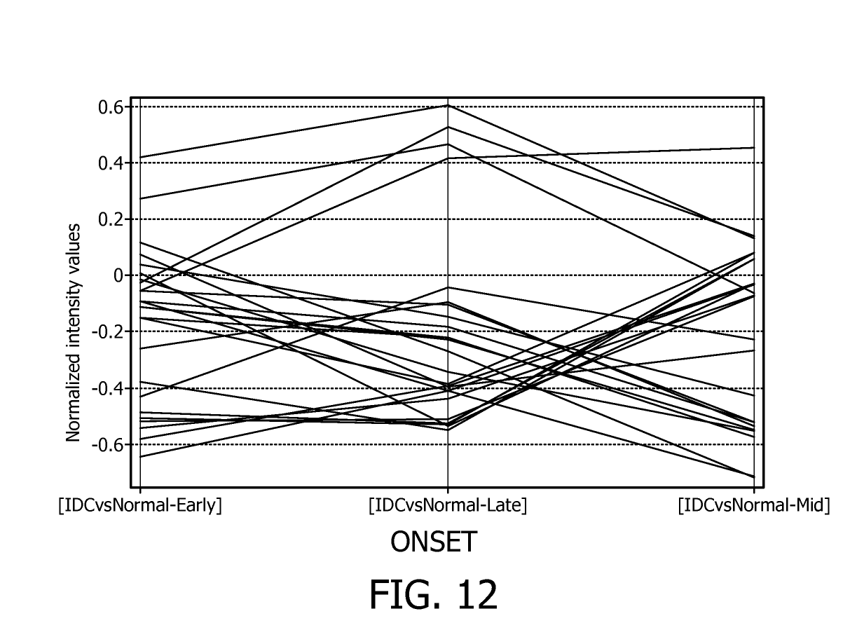

The methylation patterns at the onset of breast cancer can be used to differentiate between groups of women who would respond to therapy differently. The significant loci were screened for strong differentiators with respect to methylation levels between a set of samples from early onset patients (<40) and a set of samples for late onset patients (>50). 24 entities had a fold change of >1.3 (FIG. 12). Clustering analysis was also conducted with respect to this classification (FIG. 13).

### Example 10

### Important Pathways in Breast Cancer

We also conducted analysis to detect significant pathways using only the promoter probes (modified files) based on the 312 significant loci in total. As input, we use a table with all the probes that actually have survived the following the following steps:
1. The raw matrix is taken from the corrected signal where features are extracted (normalized) using only 5530 probes—not all probes.
2. Further, the obtained microarray data is pre-processed with Lowess intra-array normalization.
3. Quantile inter-array normalization is performed on MA matrix. For further processing M is used. (log ratio).
4. Fold change is greater than 0.7 (or less than −0.7) in at least 10 out of the 29 IDC vs. normal samples.
5. The p-value is less than 0.05 in a leave one out procedure (29 repeats where one sample is left out from the t-test). The final result table has 312 UHN ids.

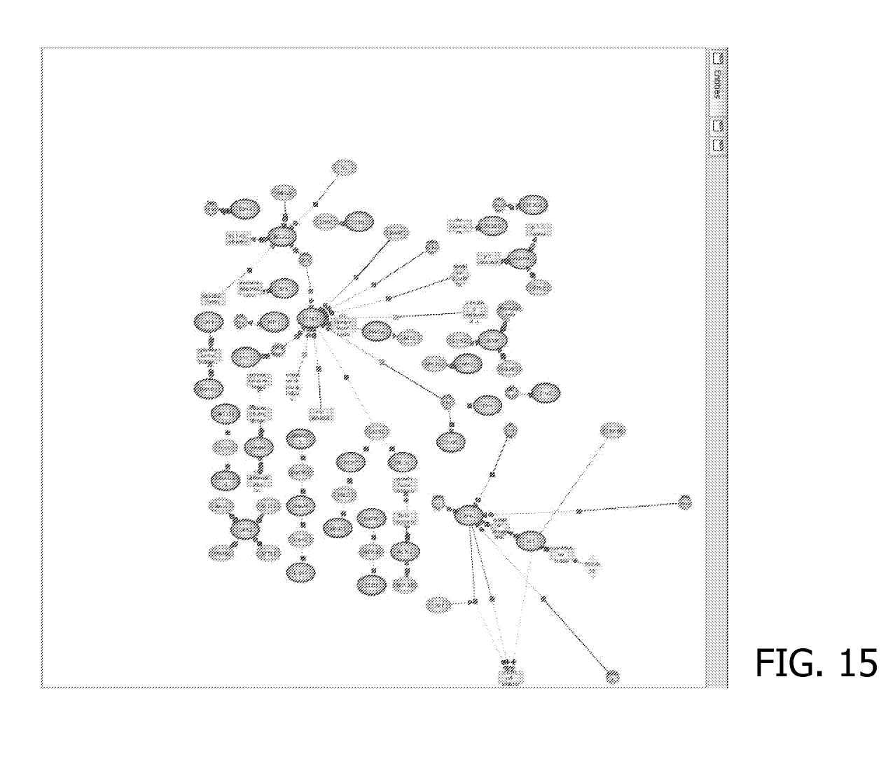

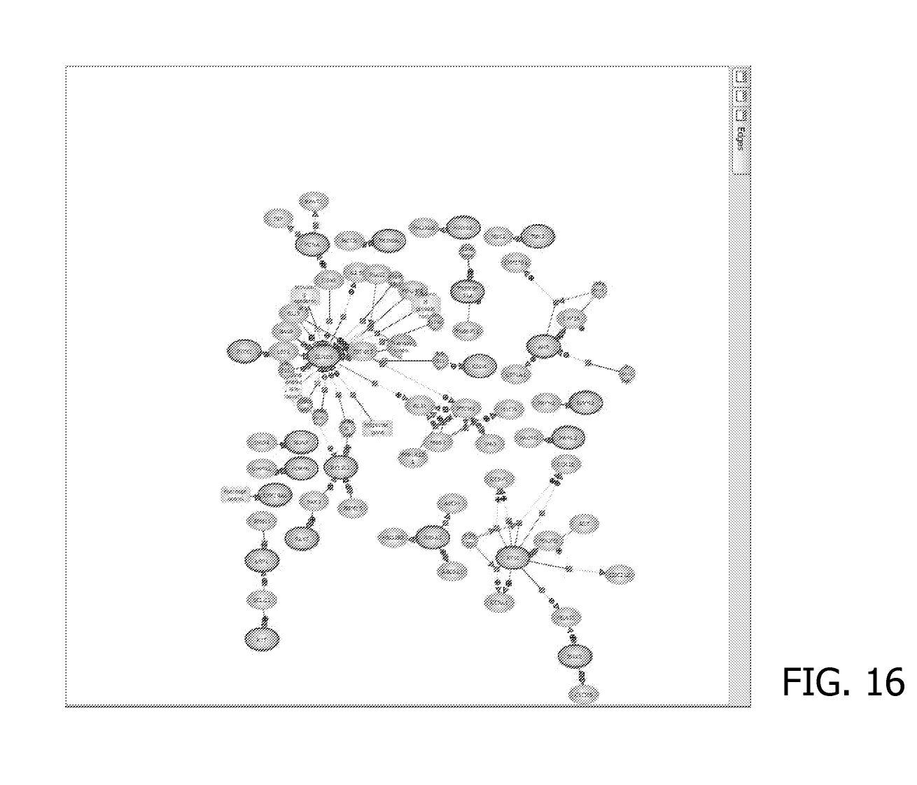

These candidate loci serve as input to the pathway analysis module in GeneSpring 10. We present the results of this analysis showing PCNA, CCND1 MAPK1, SYK as the key modifiers in our dataset FIG. 14. In FIG. 15 we show CCND1, BCL2L1, ERBB4 and PARK2 as being important hubs in the network of key regulators and targets. In FIG. 16 we see additional transcription regulators prominently showing ETS1 and AHR as being active in our sample set.

We should note that all these views can be made available in a clinical study to a clinical scientist as well as to a clinician practitioner to make an assessment of the levels of these genes in these networks so that he/she can make further decisions about the therapy plan for the patient.

TABLE 15
Sequences important in pathway analysis

| | | Gene | | | |
|---|---|---|---|---|---|
| Seq ID | ID | Symbol | State | FC | Mean |
| 71 | UHNhscpg0000434 | PCNA | down | −0.072 | 8.319 |
| 72 | UHNhscpg0005318 | PCNA | down | −0.75932 | 7.092748 |
| 73 | UHNhscpg0005042 | CCND1 | up | 0.513348 | 7.585013 |
| 74 | UHNhscpg0007998 | MAPK1 | up | 0.116532 | 7.999638 |
| 62 | UHNhscpg0004894 | SYK | up | 0.810273 | 7.966379 |
| 57 | UHNhscpg0008659 | PARK2 | up | 1.002518 | 8.169452 |
| 75 | UHNhscpg0000233 | ETS1 | down | −0.57184 | 8.788014 |
| 76 | UHNhscpg0005090 | AHR | down | −0.45214 | 8.273254 |
| 79 | UHNhscpg0004815 | ERBB4 | down | −0.08746 | 8.51624 |
| 80 | UHNhscpg0005000 | ERBB4 | down | −0.36086 | 8.728778 |
| 81 | UHNhscpg0007314 | ERBB4 | down | −0.02541 | 8.036166 |
| 82 | UHNhscpg0002306 | ERBB4 | down | −0.0647 | 8.92377 |
| 78 | UHNhscpg0005109 | BCL2L1 | up | 0.455158 | 7.859656 |

We present a list of these important pathway regulators in Table 15, where we include the fold change between IDC vs. normal and the mean value for each respective probe (ID) covering a CpG island near its respective gene. For example, SEQ ID NO 71, 72, 75, 76, 79, 80, 81, 82 which are near genes: ETS1, AHR, ERBB4 are less methylated in normal when compared to IDC (tumor), while SEQ ID NO 73, 74, 62, 57, 78 which are near genes CCND1, MAPK1, SYK, PARK2, BCL2L1 are methylated more in normal when compared to IDC (tumor).

### Applications of the Invention

The methylation status of these genes may be used for assisting in classifying infiltrating ductal carcinomas and potentially classifying them depending on their predicted prognosis.

Complete sequence list with data and SEQ ID NO's

| SEQ | | | | | |
|---|---|---|---|---|---|
| ID | | GENE | CHROMOSOME | | |
| NO | UHNID | SYMBOL | LOCATION | STRAND | DESCRIPTION |
| 1 | UHNhscpg0003204 | BDNF | chr11: 27696550-27696943 | − | brain-derived |
| | | | | | neurotrophic factor |
| 2 | UHNhscpg0000746 | CYP26A1 | chr10: 94823545-94824498 | + | cytochrome p450, |
| | | | | | family 26, |
| | | | | | subfamily a, |
| | | | | | polypeptide 1 |
| 3 | UHNhscpg0000390 | SNRPF | chr12: 94777118-94777283 | + | small nuclear |
| | | | | | ribonucleoprotein |
| | | | | | polypeptide f |
| 4 | UHNhscpg0003291 | ddb1 | chr8: 70681084-70681132 | + | sulfatase 1 |
| 5 | UHNhscpg0007521 | AB032945 | chr18: 45975419-45975817 | | hypothetical genes |
| 6 | UHNhscpg0005119 | ASNSD1 | chr2: 190234117-190234855 | + | asparagine |
| | | | | | synthetase domain |
| | | | | | containing 1 |
| 7 | UHNhscpg0005159 | BC005991 | chr6: 100069473-100070296 | − | ubiquitin specific |
| | | | | | peptidase 45 |
| 8 | UHNhscpg0005168 | C10orf11 | chr10: 77556552-77556940 | + | chromosome 10 |
| | | | | | open reading frame |
| | | | | | 11 |
| 9 | UHNhscpg0003195 | C1QTNF8 | chr16: 1078385-1078623 | − | c1q and tumor |
| | | | | | necrosis factor |
| | | | | | related protein 8 |
| 10 | UHNhscpg0007200 | CCDC93 | chr2: 118488594-118488880 | | coiled coil domain |
| | | | | | containing 93 |
| 11 | UHNhscpg0007121 | CEP350 | chr1: 178190354-178191398 | + | centrosomal |
| | | | | | protein 350 kda |
| 12 | UHNhscpg0000322 | CR596143 | chr13: 47472800-47473674 | − | succinate-CoA |
| | | | | | ligase, ADP- |
| | | | | | forming, beta |
| | | | | | subunit |
| 13 | UHNhscpg0007485 | CYP39A1 | chr6: 46728050-46729246 | − | cytochrome p450, |
| | | | | | family 39, |
| | | | | | subfamily a, |
| | | | | | polypeptide 1 |
| 14 | UHNhscpg0005129 | DAP | chr5: 10814631-10814861 | | death-associated |
| | | | | | protein |
| 15 | UHNhscpg0004955 | DUS4L | chr7: 107007599-107008461 | + | dihydrouridine |
| | | | | | synthase 4-like (s. cerevisiae) |
| 16 | UHNhscpg0000358 | GULP1 | chr2: 189015381-189015526 | + | gulp, engulfment |
| | | | | | adaptor ptb domain |
| | | | | | containing 1 |
| 17 | UHNhscpg0011146 | HADHA | chr2: 26321685-26321954 | + | hydroxyacyl- |
| | | | | | coenzyme a |
| | | | | | dehydrogenase/3- |
| | | | | | ketoacyl-coenzyme |
| | | | | | a thiolase/enoyl- |
| | | | | | coenzyme a |
| | | | | | hydratase |
| | | | | | (trifunctional |
| | | | | | protein), alpha |
| | | | | | subunit |
| 18 | UHNhscpg0000591 | LOC51057 | chr2: 63269457-63269746 | − | hypothetical |
| | | | | | protein loc51057 |
| 19 | UHNhscpg0000038 | LRP5 | chr11: 67836747-67837638 | + | low density |
| | | | | | lipoprotein |
| | | | | | receptor-related |
| | | | | | protein 5 |
| 20 | UHNhscpg0009447 | NCKIPSD | chr3: 48697708-48698578 | − | nck interacting |
| | | | | | protein with sh3 |
| | | | | | domain |
| 21 | UHNhscpg0006767 | NR4A2 | chr2: 156896978-156897265 | − | nuclear receptor |
| | | | | | subfamily 4, group |
| | | | | | a, member 2 |
| 22 | UHNhscpg0000109 | NUP93 | chr16: 55413184-55413324 | + | nucleoporin 93 kda |
| 23 | UHNhscpg0002087 | OTX1 | chr2: 63139415-63140244 | | orthodenticle |
| | | | | | homolog 1 |
| | | | | | (drosophila) |
| 24 | UHNhscpg0000193 | PCGF2 | chr17: 34157389-34157723 | − | polycomb group |
| | | | | | ring finger 2 |
| 25 | UHNhscpg0005166 | PDE6A | chr5: 149248278-149248379 | − | phosphodiesterase |
| | | | | | 6a, cgmp-specific, |
| | | | | | rod, alpha |
| 26 | UHNhscpg0004952 | RBPMS2 | chr15: 62855175-62855414 | | rna binding protein |
| | | | | | with multiple |
| | | | | | splicing 2 |
| 27 | UHNhscpg0006718 | SLC17A5 | chr6: 74420105-74420758 | − | solute carrier |
| | | | | | family 17 |
| | | | | | (anion/sugar |
| | | | | | transporter), |
| | | | | | member 5 |
| 28 | UHNhscpg0007553 | SMARCA2 | chr9: 2004804-2005843 | + | swi/snf related, |
| | | | | | matrix associated, |
| | | | | | actin dependent |
| | | | | | regulator of |
| | | | | | chromatin, |
| | | | | | subfamily a, |
| | | | | | member 2 |
| 29 | UHNhscpg0006737 | TBX3 | chr12: 113591376-113592025 | | t-box 3 (ulnar |
| | | | | | mammary |
| | | | | | syndrome) |
| 30 | UHNhscpg0005296 | TRUB2 | chr9: 130124151-130125468 | − | trub pseudouridine |
| | | | | | (psi) synthase |
| | | | | | homolog 2 (e. coli) |
| 31 | UHNhscpg0008746 | UCK2 | chr1: 164064063-164064435 | + | uridine-cytidine |
| | | | | | kinase 2 |
| 32 | UHNhscpg0006074 | ACBD3 | chr1: 224441249-224441525 | | acyl-coenzyme a |
| | | | | | binding domain |
| | | | | | containing 3 |
| 33 | UHNhscpg0007805 | ACSL3 | chr2: 223506688-223507101 | + | acyl-CoA |
| | | | | | synthetase long- |
| | | | | | chain family |
| | | | | | member 3 |
| 34 | UHNhscpg0000043 | AKT1S1 | chr19: 55071651-55072027 | − | akt1 substrate 1 |
| | | | | | (proline-rich) |
| 35 | UHNhscpg0008517 | ALG2 | chr9: 101024654-101024883 | + | asparagine-linked |
| | | | | | glycosylation 2 |
| | | | | | homolog (yeast, |
| | | | | | alpha-1,3- |
| | | | | | mannosyltransferase) |
| 36 | UHNhscpg0003749 | ANKHD1 | chr5: 139760854-139761285 | | ankyrin repeat and |
| | | | | | kh domain |
| | | | | | containing 1 |
| 37 | UHNhscpg0001513 | ANKMY2 | chr7: 16651378-16651766 | − | ankyrin repeat and |
| | | | | | mynd domain |
| | | | | | containing 2 |
| 38 | UHNhscpg0001556 | APOLD1 | chr12: 12830839-12832152 | + | apolipoprotein 1 |
| | | | | | domain containing 1 |
| 39 | UHNhscpg0000419 | ATAD5 | chr17: 26182896-26183794 | + | chrom17 origin of |
| | | | | | replication |
| 40 | UHNhscpg0006298 | BC040897 | chr9: 113433078-113433972 | − | — |
| 41 | UHNhscpg0006075 | C6orf155 | chr6: 72186425-72187545 | − | chromosome 6 |
| | | | | | open reading frame |
| | | | | | 155 |
| 42 | UHNhscpg0009610 | CHD2 | chr15: 91248245-91248931 | + | chromodomain |
| | | | | | helicase dna |
| | | | | | binding protein 2 |
| 43 | UHNhscpg0007469 | CPEB1 | chr15: 81113126-81113438 | − | cytoplasmic |
| | | | | | polyadenylation |
| | | | | | element binding |
| | | | | | protein 1 |
| 44 | UHNhscpg0008660 | DDB1 | chr11: 60856386-60857783 | − | damage-specific |
| | | | | | dna binding |
| | | | | | protein 1, 127 kda |
| 45 | UHNhscpg0007159 | DHRS13 | chr17: 24253500-24254168 | − | dehydrogenase/reductase |
| | | | | | (SDR |
| | | | | | family) member 13 |
| 46 | UHNhscpg0000452 | FAM70B | chr13: 113650943-113651734 | − | family with |
| | | | | | sequence similarity |
| | | | | | 70, member b |
| 47 | UHNhscpg0000221 | FBXL10 | chr12: 120502364-120502883 | − | F Box like protein |
| 48 | UHNhscpg0000429 | FLRT2 | chr14: 85069930-85070453 | + | fibronectin leucine |
| | | | | | rich |
| | | | | | transmembrane |
| | | | | | protein 2 |
| 49 | UHNhscpg0007607 | FLYWCH1 | chr16: 2901699-2902102 | + | zinc finger protein |
| 50 | UHNhscpg0007178 | GADD45A | chr1: 67923138-67923396 | | growth arrest and |
| | | | | | dna-damage- |
| | | | | | inducible, alpha |
| 51 | UHNhscpg0000037 | GPR89A | chr1: 144537481-144538576 | − | similar to g |
| | | | | | protein-coupled |
| | | | | | receptor 89 |
| 52 | UHNhscpg0006529 | HAND2 | chr4: 174688217-174688450 | + | basic helix-loop- |
| | | | | | helix transcription |
| | | | | | factor |
| 53 | UHNhscpg0010276 | LOC440925 | chr2: 171276912-171277222 | − | hypothetical gene |
| | | | | | supported by |
| | | | | | ak123485 |
| 54 | UHNhscpg0005089 | MALT1 | chr18: 54489095-54489924 | + | mucosa associated |
| | | | | | lymphoid tissue |
| | | | | | lymphoma |
| | | | | | translocation gene 1 |
| 55 | UHNhscpg0007662 | MORC2 | chr22: 29695224-29695365 | | morc family cw- |
| | | | | | type zinc finger 2 |
| 56 | UHNhscpg0007104 | NPY1R | chr4: 164473405-164473726 | | neuropeptide y |
| | | | | | receptor y1 |
| 57 | UHNhscpg0008659 | PARK2 | chr6: 162819158-162819373 | − | parkinson disease |
| | | | | | (autosomal |
| | | | | | recessive, juvenile) |
| | | | | | 2, parkin |
| 58 | UHNhscpg0007517, | PDE4DIP | chr1: 143643834-143644076 | − | phosphodiesterase |
| | UHNhscpg0007602 | | | | 4d interacting |
| | | | | | protein |
| | | | | | (myomegalin) |
| 59 | UHNhscpg0007487 | POLI | chr18: 50049552-50050313 | + | polymerase (dna |
| | | | | | directed) iota |
| 60 | UHNhscpg0009180 | PSMB7 | chr9: 126217209-126217803 | − | proteasome |
| | | | | | (prosome, |
| | | | | | macropain) |
| | | | | | subunit, beta type, 7 |
| 61 | UHNhscpg0003180 | SIX3 | chr2: 45020740-45020934 | | sine oculis |
| | | | | | homeobox |
| | | | | | homolog 3 |
| | | | | | (drosophila) |
| 62 | UHNhscpg0004894 | SYK | chr9: 92603346-92603864 | | spleen tyrosine |
| | | | | | kinase |
| 63 | UHNhscpg0000364 | TES | chr7: 115637345-115637985 | + | testis derived |
| | | | | | transcript (3 lim |
| | | | | | domains) |
| 64 | UHNhscpg0000227 | TJP1 | chr15: 28270526-28271354 | − | tight junction |
| | | | | | protein |
| 65 | UHNhscpg0000085 | TNFRSF13B | chr17: 16802068-16802226 | − | tumor necrosis |
| | | | | | factor receptor |
| | | | | | superfamily 13 B |
| 66 | UHNhscpg0000204 | TTC23 | chr15: 97608595-97609633 | − | Hypothetical |
| | | | | | protein FLJ13168. |
| 67 | UHNhscpg0000299 | ZCSL2 | chr3: 16281447-16281734 | + | DPH3, KTI11 |
| | | | | | homolog (S. cerevisiae) |
| 68 | UHNhscpg0007132 | ZDHHC20 | chr13: 20930805-20931472 | − | zinc finger, dhhc- |
| | | | | | type containing 20 |
| 69 | UHNhscpg0007446 | ZHX2 | chr8: 123862942-123863095 | + | zinc fingers and |
| | | | | | homeoboxes 2 |
| 70 | UHNhscpg0003020 | ZNF786 | chr7: 148418255-148419867 | − | zinc finger protein |
| | | | | | ZNF786 |
| 71 | UHNhscpg0000434 | PCNA | chr20: 5048602-5049085 | − | proliferating cell |
| | | | | | nuclear antigen |
| 72 | UHNhscpg0005318 | PCNA | chr20: 5055093-5055277 | − | proliferating cell |
| | | | | | nuclear antigen |
| 73 | UHNhscpg0005042 | CCND1 | chr11: 69162738-69163538 | + | cyclin D1 |
| 74 | UHNhscpg0007998 | MAPK1 | chr22: 20551323-20552175 | − | mitogen-activated |
| | | | | | protein kinase 1 |
| 75 | UHNhscpg0000233 | ETS1 | chr11: 127896681-127897162 | − | ETS1 protein. |
| 76 | UHNhscpg0005090 | AHR | chr7: 17326397-17326537 | + | arylhydrocarbon |
| | | | | | receptor repressor |
| 77 | UHNhscpg0003170 | ESR2 | chr14: 63831062-63831529 | − | 3pv2. |
| 78 | UHNhscpg0005109 | BCL2L1 | chr20: 29774490-29774701 | − | BCL2-like 12 |
| | | | | | isoform 1 |
| 79 | UHNhscpg0004815 | ERBB4 | chr2: 212526356-212526416 | − | v-erb-a |
| | | | | | erythroblastic |
| | | | | | leukemia viral |
| | | | | | oncogene |
| 80 | UHNhscpg0005000 | ERBB4 | chr2: 212552939-212553004 | − | v-erb-a |
| | | | | | erythroblastic |
| | | | | | leukemia viral |
| | | | | | oncogene |
| 81 | UHNhscpg0007314 | ERBB4 | chr2: 212713502-212713610 | − | v-erb-a |
| | | | | | erythroblastic |
| | | | | | leukemia viral |
| | | | | | oncogene |
| 82 | UHNhscpg0002306 | ERBB4 | chr2: 213109241-213109694 | − | v-erb-a |
| | | | | | erythroblastic |
| | | | | | leukemia viral |
| | | | | | oncogene |
| 83 | UHNhscpg0007411 | TMEM117 | chr12: 42519746-42519891 | + | hypothetical |
| | | | | | protein LOC84216 |
| 84 | UHNhscpg0008515 | GALNT13 | chr2: 154892928-154892960 | + | UDP-N-acetyl- |
| | | | | | alpha-D- |
| | | | | | galactosamine:poly |
| | | | | | peptide |
| 85 | UHNhscpg0008264 | BDNF | chr11: 27700616-27701448 | − | brain-derived |
| | | | | | neurotrophic factor |
| | | | | | isoform b |
| 86 | UHNhscpg0002632 | DUSP4 | chr8: 29265449-29265864 | − | dual specificity |
| | | | | | phosphatase 4 |
| | | | | | isoform 1 |
| 87 | UHNhscpg0006957 | KIAA0776 | chr6: 96969405-96969504 | + | hypothetical |
| | | | | | protein LOC23376 |
| 88 | UHNhscpg0008950 | NME6 | chr3: 48342609-48343351 | − | “non-metastatic |
| | | | | | cells 6, protein |
| | | | | | expressed in |
| | | | | | (nucleoside- |
| | | | | | diphosphate |
| | | | | | kinase)” |
| 89 | UHNhscpg0000024 | SMG6 | chr17: 2125839-2125862 | − | Est1p-like protein A |
| 90 | UHNhscpg0010841 | ABCB10 | chr1: 229693478-229694354 | − | “ATP-binding |
| | | | | | cassette, sub- |
| | | | | | family B, member |
| | | | | | 10” |
| 91 | UHNhscpg0010601 | MMP25 | chr16: 3095712-3095935 | + | matrix |
| | | | | | metalloproteinase |
| | | | | | 25 preproprotein |
| 92 | UHNhscpg0011399 | LNPEP | chr5: 96352319-96352368 | + | leucyl/cystinyl |
| | | | | | aminopeptidase |
| | | | | | isoform 1 |
| 93 | UHNhscpg0000636 | LRRC4C | chr11: 40283867-40284519 | − | netrin-G1 ligand |
| 94 | UHNhscpg0007219 | HSPA2 | chr14: 65006815-65006989 | + | heat shock 70 kDa |
| | | | | | protein 2 |
| 95 | UHNhscpg0001461 | ROBO3 | chr11: 124736261-124736800 | + | “roundabout, axon |
| | | | | | guidance receptor, |
| | | | | | homolog 3” |
| 96 | UHNhscpg0005839 | DFNB31 | chr9: 117261407-117261543 | − | OTTHUMP00000021976. |
| 97 | UHNhscpg0010619 | PGD | chr1: 10458486-10458639 | + | phosphogluconate |
| | | | | | dehydrogenase |
| 98 | UHNhscpg0004672 | PVRL3 | chr3: 110789616-110790285 | + | PVRL3 protein. |
| 99 | UHNhscpg0004504 | GAPDH | chr12: 6519633-6520564 | + | Glyceraldehyde-3- |
| | | | | | phosphate |
| | | | | | dehydrogenase(EC |
| | | | | | 1.2.1.12) |
| | | | | | (Fragment). |
| 100 | UHNhscpg0000914 | AF271776 | chrM: 7586-8094 | + | ATP synthase a |
| | | | | | chain (EC |
| | | | | | 3.6.3.14) (ATPase |
| | | | | | protein 6). |
| 101 | UHNhscpg0000024 | SMG6 | chr17: 2125839-2125862 | − | Est1p-like protein A |
| 102 | UHNhscpg0000230 | DLX6 | chr7: 96477436-96477749 | + | distal-less |
| | | | | | homeobox 6 |
| 103 | UHNhscpg0007777 | IFT88 | chr13: 21140610-21140861 | − | intraflagellar |
| | | | | | transport 88 |
| | | | | | homologue |
| | | | | | isoform 1 |
| 104 | UHNhscpg0000501 | SLC13A3 | chr20: 45204611-45205384 | − | solute carrier |
| | | | | | family 13 member |
| | | | | | 3 isoform A |
| 105 | UHNhscpg0007046 | IREB2 | chr15: 78730311-78731340 | + | iron responsive |
| | | | | | element binding |
| | | | | | protein 2 |
| 106 | UHNhscpg0008329 | RTTN | chr18: 67872498-67872926 | − | rotatin |
| 107 | UHNhscpg0000211 | KIAA1530 | chr4: 1340633-1341615 | + | KIAA1530 protein |
| 108 | UHNhscpg0002300 | PSIP1 | chr9: 15509859-15509960 | − | PC4 and SFRS1 |
| | | | | | interacting protein |
| | | | | | 1 isoform 2 |
| 109 | UHNhscpg0004523 | CR601508 | chr6: 52761939-52762111 | − | OTTHUMP00000016614 |
| 110 | UHNhscpg0009237 | BANK1 | chr4: 102711507-102712443 | + | hypothetical |
| | | | | | protein FLI34204 |
| 111 | UHNhscpg0006618 | JAK2 | chr9: 4984202-4984895 | + | janus kinase 2 |

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. (canceled)

2. A method for assisting in classifying a breast cancer disorder, comprising the steps of:
providing a sample from a subject to be analyzed, wherein said sample is provided outside the human or animal body,
determining a methylation status for one or more sequences according to SEQ ID NO:1-111.

3. The method according to claim 2, further comprising
a) the one or more results from the methylation status test is input into a classifier that is obtained from a Multi Variate Model,
b) calculating a likelihood as to whether the sample is from a normal breast tissue, infiltrating ductal carcinoma (IDC) or a benign breast tumor.

4. The method according to claim 2, further comprising determining at least one parameter in a sample obtained from said subject, said parameter being the expression level of at least one of the following proteins selected from the group consisting of Estrogen Receptor (ER), Progesterone receptor (PR) and Herceptin (HER2) in said sample.

5. The method according claim 3, for assisting in the determining whether a sample is an infiltrating ductal carcinoma or a normal sample,
wherein the HER2 status is determined in a sample, and
wherein the methylation status is determined for at least LRRC4C, HSPA2, ROBO3, AF271776, DENB31, PGD (SEQ ID NO: 93, 94, 95, 100, 96, and 97).

6. The method according to claim 3, for assisting in the determining whether a sample is an infiltrating ductal carcinoma or a normal sample,
wherein the ER status is determined in a sample, and
wherein the methylation status is determined for at least LRRC4C, KIAA0776, NME6, SMG6, ABCB10, MMP25 and LNPEP (SEQ. ID NO: 93, 87, 88, 89, 90, 91 and 92)

7. The method according to claim 2, for assisting in the determining whether a sample is an infiltrating ductal carcinoma or a normal sample,
wherein the premenopausal status of said subject is determined, and
wherein the methylation status is determined for at least TMEM117, GALNT13, BDNF, and DUSP4 [SEQ ID NO 83, 84, 85, 86].

8. The method according to claim 3, for assisting in the determining whether a sample is an infiltrating ductal carcinoma or a normal sample,
wherein the ER status, the PR status and the Her2 status is determined in a sample, and
wherein the methylation status is determined for LRRC4C PVRL3, ROBO3, AF271776, SMG6, AF271776, ABCB10 (SEQ ID NO, 93, 95, 100, 89, and 90).

9. The method according to claim 3, for assisting in the determining whether the sample is from a infiltrating ductal carcinoma or benign breast cancer tumor, wherein the methylation status is determined for IFT88, SLC13A3, IREB2, RTTN, KIAA1530, PSIP1, CR601508, BANK1, JAK2 (SEQ ID NO: 103, 104, 105, 106, 107, 108, 109, 110, 111 and respectively).

10. The method according to claim 2, for assisting in the determining whether a sample is an invasive ductal carcinoma or normal, wherein the methylation status is determined for at least ddb1 (SEQ ID NO:4), DDB1 (SEQ ID NO: 44), DAP (SEQ. ID NO:14), TBX3 (SEQ ID NO:29), LRP5 (SEQ ID NO:19) and PCGF2 (SEQ ID NO:24).

11. The method according to claim 2, for assisting in determining whether a sample is an invasive ductal carcinoma or a normal sample, wherein the methylation is determined for at least 10 sequences selected from the group consisting of: SEQ ID NO: 15 DUS4L, 27 SLC17A5, 21 NR4A2, 20 NCKIPSD, 57 PARK2, 2 CYT26A1, 44 DDB1, 58 PDE4DIP, 14 DAP, 29 TBX3, 19 LRP5, 16 GULP1, 64 TJP1, 25 PDE6A, 67 ZCSL2, 22 NUP93, 12 CR596143, 24 PCGF2, 3 SNRPF, 1.8 L0051057, and 8 C10orf11.

12. The method according to claim 2, for assisting in determining whether a sample is an invasive ductal carcinoma or a normal sample, wherein the methylation is determined for at least PCNA, CCND1 MAPK1, SYK (SEQ ID NO 71, 72, 73, 74, 62), BCL2L1, ERBB4 and PARK2 (SEC ID NO 78, 79, 80, 81, 82, 57), ETS1 and AHR (SEQ ID NO: 75, 76).

13. The method according to claim 2, wherein the methylation status is determined by means of one or more of the methods selected form the group of,
a. bisulfite sequencing
b. pyrosequencing
c. methylation-sensitive single-strand conformation analysis(MS-SSCA)
d. high resolution melting analysis (HRM)
e. methylation-sensitive single nucleotide primer extension (MS-SnuPE)
f. base-specific cleavage/MALDI-TOF
g. methylation-specific FOR (MSP)
h. microarray-based methods and
i. msp I cleavage.
j. Methylation sensitive sequencing

14. The method according to claim 2, wherein the sample to be analyzed is from a tissue type selected from the group of tissues such as, a tissue biopsy from the tissue to be analyzed, tumor tissue, body fluids, blood, serum, saliva and urine.

15. The method according to claim 2, wherein the methylation pattern obtained is used to predict the therapeutic response to the treatment of a breast cancer.

16. Composition or array comprising nucleic acids with sequences which are identical to at least 10 of the sequences according to SEQ ID NO: 1-111 for use in a method for assisting in classifying a breast cancer disorder.

17. Composition or array according to claim 15 for use in a method for assisting in classifying a breast cancer disorder, comprising nucleic acids with sequences which are identical to ddb1 (SEC ID NO:4), DDB1 (SEC ID NO 44), DAP (SEQ ID NO:14), TBX3 (SEQ ID NO:29), LRP5 (SEQ ID NO:19) and PCGF2 (SEQ ID NO:24).

18. A computer program product being adapted to enable a computer system comprising at least one computer having a data storage means associated therewith to operate a processor arranged for carrying out a method according to claim 14.
